SEQUENCE LISTING (104 sequences)

SEQ ID NO: 1 - protein, 10 aa, Artificial Sequence
  synthetic construct
GYTFTNYVIH

SEQ ID NO: 2 - protein, 10 aa, Artificial Sequence
  synthetic construct
GYSFTNYYIH

SEQ ID NO: 3 - protein, 10 aa, Artificial Sequence
  synthetic construct
GYTFTNYWIH

SEQ ID NO: 4 - protein, 17 aa, Artificial Sequence
  synthetic construct
YIYPYNDGILYNEKFKG

SEQ ID NO: 5 - protein, 17 aa, Artificial Sequence
  synthetic construct
YIDPLNGDTTYNQKFKG

SEQ ID NO: 6 - protein, 17 aa, Artificial Sequence
  synthetic construct
YTDPRTDYTEYNQKFKD

SEQ ID NO: 7 - protein, 8 aa, Artificial Sequence
  synthetic construct
GGYYVPDY

SEQ ID NO: 8 - protein, 8 aa, Artificial Sequence
  synthetic construct
GGYYVYDY

SEQ ID NO: 9 - protein, 8 aa, Artificial Sequence
  synthetic construct
GGKRAMDY

SEQ ID NO: 10 - protein, 8 aa, Artificial Sequence
  synthetic construct
GGRVGLGY

SEQ ID NO: 11 - protein, 16 aa, Artificial Sequence
  synthetic construct
RSRQSIVHTNGNTYLG

SEQ ID NO: 12 - protein, 11 aa, Artificial Sequence
  synthetic construct
RASQDISNYLN

SEQ ID NO: 13 - protein, 11 aa, Artificial Sequence
  synthetic construct
RASQSISNYLN

SEQ ID NO: 14 - protein, 16 aa, Artificial Sequence
  synthetic construct
RSSQNIVQSNGNTYLE

SEQ ID NO: 15 - protein, 7 aa, Artificial Sequence
  synthetic construct
KVSNRFS

SEQ ID NO: 16 - protein, 7 aa, Artificial Sequence
  synthetic construct
YTSRLYS

SEQ ID NO: 17 - protein, 7 aa, Artificial Sequence
  synthetic construct
KVFHRFS

SEQ ID NO: 18 - protein, 9 aa, Artificial Sequence
  synthetic construct
FQGSHVPYT

SEQ ID NO: 19 - protein, 9 aa, Artificial Sequence
  synthetic construct
QQGNTLPWT

SEQ ID NO: 20 - protein, 9 aa, Artificial Sequence
  synthetic construct
FQGSHVPWT

SEQ ID NO: 21 - protein, 117 aa, Artificial Sequence
  synthetic construct
EVQLQQSGPELVKPGASVKMSCKASGYTFTNYVIHWVKRRPGQGLEWIGYIYPYNDGILYNEKFKGKATVTSDKSSSTAYMDLSSLTSEDSAVYYCTRGGYYVPDYWGQGTTLTVSS

SEQ ID NO: 22 - protein, 117 aa, Artificial Sequence
  synthetic construct
EVQLQQSGPELVKPGASVKMSCKASGYTFTNYVIHWVKRRPGQGLEWIGYIYPYNDGILYNEKFKGKATVTSDKSSSTAYMDLSSLTSEDSAVYYCTRGGYYVPDYWGQGTLVTVSS

SEQ ID NO: 23 - protein, 117 aa, Artificial Sequence
  synthetic construct
QVQLVQSGAEVKKPGASVQVSCKASGYTFTNYVIHWLRQAPGQGLEWMGYIYPYNDGILYNEKFKGRVTMTSDTSISTAYMELSSLRSDDTAVYYCARGGYYVPDYWGQATLVTVSS

SEQ ID NO: 24 - protein, 117 aa, Artificial Sequence
  synthetic construct
QVQLVQSGAEVKKPGASVQVSCKASGYTFTNYVIHWLRQAPGQGLEWMGYIYPYNDGILYNEKFKGRVTMTSDTSISTAYMELSSLRSDDTAVYYCARGGYYVYDYWGQATLVTVSS

SEQ ID NO: 25 - protein, 117 aa, Artificial Sequence
  synthetic construct
EVQLVQSGAEVKKPGATVKISCKVSGYTFTNYVIHWVQQAPGKGLEWMGYIYPYNDGILYNEKFKGRVTITADTSTDTAYMELSSLRSEDTAVYYCATGGYYVPDYWGQGTTVTVSS

SEQ ID NO: 26 - protein, 117 aa, Artificial Sequence
  synthetic construct
EVQLVQSGAEVKKPGESLKISCKGSGYTFTNYVIHWVRQMPGKGLEWMGYIYPYNDGILYNEKFKGQVTISADKSISTAYLQWSSLKASDTAMYYCARGGYYVPDYWGQGTTVTVSS

SEQ ID NO: 27 - protein, 117 aa, Artificial Sequence
  synthetic construct
QVQLVQSGAEVKKPGASVKVSCKASGYTFTNYVIHWVRQAPGQGLEWMGYIYPYNDGILYNEKFKGRVTMTTDTSTSTAYMELRSLRSDDTAVYYCARGGYYVPDYWGQGTTVTVSS

SEQ ID NO: 28 - protein, 117 aa, Artificial Sequence
  synthetic construct
EVQLQQSGPELMKPGASVKISCKASGYSFTNYYIHWVNQSHGKSLEWIGYIDPLNGDTTYNQKFKGKATLTVDKSSSTAYMRLSSLTSADSAVYYCARGGKRAMDYWGQGTSVTVSS

SEQ ID NO: 29 - protein, 117 aa, Artificial Sequence
  synthetic construct
QVQLVQSGAEVKKPGASVKVSCKASGYSFTNYYIHWVRQAPGQGLEWMGYIDPLNGDTTYNQKFKGRVTMTRDTSTSTVYMELSSLRSEDTAVYYCARGGKRAMDYWGQGTLVTVSS

SEQ ID NO: 30 - protein, 117 aa, Artificial Sequence
  synthetic construct
QVQLVQSGAEVKKPGSSVKVSCKASGYSFTNYYIHWVRQAPGQGLEWMGYIDPLNGDTTYNQKFKGRVTITADESTSTAYMELSSLRSEDTAVYYCARGGKRAMDYWGQGTLVTVSS

SEQ ID NO: 31 - protein, 117 aa, Artificial Sequence
  synthetic construct
EVQLVQSGAEVKKPGESLKISCKGSGYSFTNYYIHWVRQMPGKGLEWMGYIDPLNGDTTYNQKFKGQVTISADKSISTAYLQWSSLKASDTAMYYCARGGKRAMDYWGQGTLVTVSS

SEQ ID NO: 32 - protein, 117 aa, Artificial Sequence
  synthetic construct
QVQLVQSGAEVKKPGSSVKVSCKASGYSFTNYYIHWVRQAPGQGLEWMGYIDPLNGDTTYAQKFQGRVTITADESTSTAYMELSSLRSEDTAVYYCARGGKRAMDYWGQGTLVTVSS

SEQ ID NO: 33 - protein, 117 aa, Artificial Sequence
  synthetic construct
EVQLVQSGAEVKKPGESLKISCKGSGYSFTNYYIHWVRQMPGKGLEWMGYIDPLNGDTTYSPSFQGQVTISADKSISTAYLQWSSLKASDTAMYYCARGGKRAMDYWGRGTLVTVSS

SEQ ID NO: 34 - protein, 117 aa, Artificial Sequence
  synthetic construct
QVQLVQSGAEVKKPGASVQVSCKASGYSFTNYYIHWLRQAPGQGLEWMGYIDPLNGDTTYNQKFKGRVTMTSDTSISTAYMELSSLRSDDTAVYYCARGGKRAMDYWGQATLVTVSS

SEQ ID NO: 35 - protein, 117 aa, Artificial Sequence
  synthetic construct
QVQLQQFGAELAKPGASVQMSCKASGYTFTNYWIHWVKQRPGQGLEWIGYTDPRTDYTEYNQKFKDKATLAADRSSSTAYMRLSSLTSEDSAVYYCAGGGRVGLGYWGHGSSVTVSS

SEQ ID NO: 36 - protein, 117 aa, Artificial Sequence
  synthetic construct
QVQLVQSGAEVKKPGASVKVSCKASGYTFTNYWIHWVRQAPGQGLEWMGYTDPRTDYTEYNQKFKDRVTMTRDTSTSTVYMELSSLRSEDTAVYYCARGGRVGLGYWGQGTLVTVSS

SEQ ID NO: 37 - protein, 117 aa, Artificial Sequence
  synthetic construct
QVQLVQSGAEVKKPGSSVKVSCKASGYTFTNYWIHWVRQAPGQGLEWMGYTDPRTDYTEYNQKFKDRVTITADESTSTAYMELSSLRSEDTAVYYCARGGRVGLGYWGQGTLVTVSS

SEQ ID NO: 38 - protein, 117 aa, Artificial Sequence
  synthetic construct
EVQLVQSGAEVKKPGESLKISCKGSGYTFTNYWIHWVRQMPGKGLEWMGYTDPRTDYTEYNQKFKDQVTISADKSISTAYLQWSSLKASDTAMYYCARGGRVGLGYWGQGTLVTVSS

SEQ ID NO: 39 - protein, 117 aa, Artificial Sequence
  synthetic construct
QVQLVQSGAEVKKPGSSVKVSCKASGYTFTNYWIHWVRQAPGQGLEWMGYTDPRTDYTEYAQKFQGRVTITADESTSTAYMELSSLRSEDTAVYYCARGGRVGLGYWGQGTLVTVSS

SEQ ID NO: 40 - protein, 117 aa, Artificial Sequence
  synthetic construct
EVQLVQSGAEVKKPGESLKISCKGSGYTFTNYWIHWVRQMPGKGLEWMGYTDPRTDYTEYSPSFQGQVTISADKSISTAYLQWSSLKASDTAMYYCARGGRVGLGYWGQGTLVTVSS

SEQ ID NO: 41 - protein, 112 aa, Artificial Sequence
  synthetic construct
DVLMTQTPLSLPVNLGDQASISCRSRQSIVHTNGNTYLGWFLQKPGQSPKLLIYKVSNRFSGVPDRFSGSGSGTDFTLTISRVEAEDLGVYYCFQGSHVPYTFGGGTKLEIK

SEQ ID NO: 42 - protein, 112 aa, Artificial Sequence
  synthetic construct
DVLMTQTPLSLPVNLGDQASISCRSRQSIVHTNGNTYLGWFLQKPGQSPKLLIYKVSNRFSGVPDRFSGSGSGTDFTLTISRVEAEDLGVYYCFQGSHVPYTFGQGTKVEIK

SEQ ID NO: 43 - protein, 112 aa, Artificial Sequence
  synthetic construct
DIVMTQSPLSLPVTPGEPASISCRSRQSIVHTNGNTYLGWYLQKPGQSPRLLIYKVSNRFSGVPDRFSGSGSGTDFTLKISRVEADDVGIYYCFQGSHVPYTFGQGTKLEIK

SEQ ID NO: 44 - protein, 112 aa, Artificial Sequence
  synthetic construct
DVVMTQSPLSLPVTLGQPASISCRSRQSIVHTNGNTYLGWFQQRPGQSPRRLIYKVSNRFSGVPDRFSGSGSGTDFTLKISRVEAEDVGVYYCFQGSHVPYTFGQGTKLEIK

SEQ ID NO: 45 - protein, 112 aa, Artificial Sequence
  synthetic construct
DIVMTQSPDSLAVSLGERATINCRSRQSIVHTNGNTYLGWYQQKPGQPPKLLIYKVSNRFSGVPDRFSGSGSGTDFTLTISSLQAEDVAVYYCFQGSHVPYTFGQGTKLEIK

SEQ ID NO: 46 - protein, 107 aa, Artificial Sequence
  synthetic construct
DIQMTQTTSSLSASLGDRVTISCRASQDISNYLNWYQQKPDGTVKLLIYYTSRLYSGVPSRFSGSGSGTDYSLTISNLEQEDIATYFCQQGNTLPWTFGGGTKLEIK

SEQ ID NO: 47 - protein, 107 aa, Artificial Sequence
  synthetic construct
DIQMTQSPSSLSASVGDRVTITCRASQDISNYLNWYQQKPGKAPKLLIYYTSRLYSGVPSRFSGSGSGTDFTFTISSLQPEDIATYYCQQGNTLPWTFGQGTKVEIK

SEQ ID NO: 48 - protein, 107 aa, Artificial Sequence
  synthetic construct
DIQMTQSPSSLSASVGDRVTITCRASQSISNYLNWYQQKPGKAPKLLIYYTSRLYSGVPSRFSGSGSGTDFTLTISSLQPEDFATYYCQQGNTLPWTFGQGTKVEIK

SEQ ID NO: 49 - protein, 107 aa, Artificial Sequence
  synthetic construct
DIVMTQSPLSLPVTPGEPASISCRASQDISNYLNWYLQKPGQSPRLLIYYTSRLYSGVPDRFSGSGSGTDFTLKISRVEADDVGIYYCQQGNTLPWTFGQGTKLEIK

SEQ ID NO: 50 - protein, 112 aa, Artificial Sequence
  synthetic construct
DVFMTQTPLSLPVSLGDQASISCRSSQNIVQSNGNTYLEWYLQKPGQSPKLLIYKVFHRFSGVPDRFSGSGSGTDFTLKISRVEAEDLGVYYCFQGSHVPWTFGGGTKVEIK

SEQ ID NO: 51 - protein, 112 aa, Artificial Sequence
  synthetic construct
DVVMTQSPLSLPVTLGQPASISCRSSQNIVQSNGNTYLEWFQQRPGQSPRRLIYKVFHRFSGVPDRFSGSGSGTDFTLKISRVEAEDVGVYYCFQGSHVPYTFGQGTKLEIK

SEQ ID NO: 52 - protein, 112 aa, Artificial Sequence
  synthetic construct
DIVMTQSPDSLAVSLGERATINCRSSQNIVQSNGNTYLEWYQQKPGQPPKLLIYKVFHRFSGVPDRFSGSGSGTDFTLTISSLQAEDVAVYYCFQGSHVPYTFGQGTKLEIK

SEQ ID NO: 53 - protein, 330 aa, Artificial Sequence [sequence omitted]

SEQ ID NO: 54 - protein, 330 aa, Artificial Sequence
  synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 326 aa, Artificial Sequence
  synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 56 - protein, 377 aa, Artificial Sequence
  synthetic construct
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 57 - protein, 326 aa, Artificial Sequence
  synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 58 - protein, 326 aa, Artificial Sequence
  synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 59 - protein, 327 aa, Artificial Sequence
  synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 60 - protein, 107 aa, Artificial Sequence [sequence omitted]

SEQ ID NO: 61 - protein, 329 aa, Artificial Sequence
  synthetic construct
ARTTAPSVYPLVPGCSGTSGSLVTLGCLVKGYFPEPVTVKWNSGALSSGVHTFPAVLQSGLYTLSSSVTVPSSTWSSQTVTCSVAHPATKSNLIKRIEPRRPKPRPPTDICSCDDNLGRPSVFIFPPKPKDILMITLTPKVTCVVVDVSEEEPDVQFSWFVDNVRVFTAQTQPHEEQLNGTFRVVSTLHIQHQDWMSGKEFKCKVNNKDLPSPIEKTISKPRGKARTPQVYTIPPPREQMSKNKVSLTCMVTSFYPASISVEWERNGELEQDYKNTLPVLDSDESYFLYSKLSVDTDSWMRGDIYTCSVVHEALHNHHTQKNLSRSPGK

SEQ ID NO: 62 - protein, 107 aa, Artificial Sequence
  synthetic construct
RADAAPTVSIFPPSMEQLTSGGATVVCFVNNFYPRDISVKWKIDGSEQRDGVLDSVTDQDSKDSTYSMSSTLSLTKVEYERHNLYTCEVVHKTSSSPVVKSFNRNEC

SEQ ID NO: 63 - protein, 323 aa, Artificial Sequence
  synthetic construct
GQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 64 - protein, 104 aa, Artificial Sequence
  synthetic construct
RDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 65 - protein, 293 aa, Artificial Sequence
  synthetic construct
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVE

SEQ ID NO: 66 - protein, 219 aa, Artificial Sequence
  synthetic construct
DVLMTQTPLSLPVNLGDQASISCRSRQSIVHTNGNTYLGWFLQKPGQSPKLLIYKVSNRFSGVPDRFSGSGSGTDFTLTISRVEAEDLGVYYCFQGSHVPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 219 aa, Artificial Sequence
  synthetic construct
DVLMTQTPLSLPVNLGDQASISCRSRQSIVHTNGNTYLGWFLQKPGQSPKLLIYKVSNRFSGVPDRFSGSGSGTDFTLTISRVEAEDLGVYYCFQGSHVPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 219 aa, Artificial Sequence
  synthetic construct
DIVMTQSPLSLPVTPGEPASISCRSRQSIVHTNGNTYLGWYLQKPGQSPRLLIYKVSNRFSGVPDRFSGSGSGTDFTLKISRVEADDVGIYYCFQGSHVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 214 aa, Artificial Sequence
  synthetic construct
DIVMTQSPLSLPVTPGEPASISCRASQDISNYLNWYLQKPGQSPRLLIYYTSRLYSGVPDRFSGSGSGTDFTLKISRVEADDVGIYYCQQGNTLPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - protein, 219 aa, Artificial Sequence
  synthetic construct
DIVMTQSPDSLAVSLGERATINCRSSQNIVQSNGNTYLEWYQQKPGQPPKLLIYKVFHRFSGVPDRFSGSGSGTDFTLTISSLQAEDVAVYYCFQGSHVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - protein, 214 aa, Artificial Sequence
  synthetic construct
DIQMTQSPSSLSASVGDRVTITCRASQSISNYLNWYQQKPGKAPKLLIYYTSRLYSGVPSRFSGSGSGTDFTLTISSLQPEDFATYYCQQGNTLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - protein, 219 aa, Artificial Sequence
  synthetic construct
DVVMTQSPLSLPVTLGQPASISCRSRQSIVHTNGNTYLGWFQQRPGQSPRRLIYKVSNRFSGVPDRFSGSGSGTDFTLKISRVEAEDVGVYYCFQGSHVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 219 aa, Artificial Sequence
  synthetic construct
DVVMTQSPLSLPVTLGQPASISCRSSQNIVQSNGNTYLEWFQQRPGQSPRRLIYKVFHRFSGVPDRFSGSGSGTDFTLKISRVEAEDVGVYYCFQGSHVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - protein, 214 aa, Artificial Sequence
  synthetic construct
DIQMTQSPSSLSASVGDRVTITCRASQDISNYLNWYQQKPGKAPKLLIYYTSRLYSGVPSRFSGSGSGTDFTFTISSLQPEDIATYYCQQGNTLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 219 aa, Artificial Sequence
  synthetic construct
DVFMTQTPLSLPVSLGDQASISCRSSQNIVQSNGNTYLEWYLQKPGQSPKLLIYKVFHRFSGVPDRFSGSGSGTDFTLKISRVEAEDLGVYYCFQGSHVPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - protein, 444 aa, Artificial Sequence
  synthetic construct
EVQLQQSGPELVKPGASVKMSCKASGYTFTNYVIHWVKRRPGQGLEWIGYIYPYNDGILYNEKFKGKATVTSDKSSSTAYMDLSSLTSEDSAVYYCTRGGYYVPDYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 77 - protein, 444 aa, Artificial Sequence
  synthetic construct
QVQLVQSGAEVKKPGASVQVSCKASGYTFTNYVIHWLRQAPGQGLEWMGYIYPYNDGILYNEKFKGRVTMTSDTSISTAYMELSSLRSDDTAVYYCARGGYYVPDYWGQATLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 78 - protein, 444 aa, Artificial Sequence
  synthetic construct
QVQLVQSGAEVKKPGASVQVSCKASGYSFTNYYIHWLRQAPGQGLEWMGYIDPLNGDTTYNQKFKGRVTMTSDTSISTAYMELSSLRSDDTAVYYCARGGKRAMDYWGQATLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 79 - protein, 443 aa, Artificial Sequence
  synthetic construct
QVQLVQSGAEVKKPGASVKVSCKASGYTFTNYWIHWVRQAPGQGLEWMGYTDPRTDYTEYNQKFKDRVTMTRDTSTSTVYMELSSLRSEDTAVYYCARGGRVGLGYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 80 - protein, 443 aa, Artificial Sequence
  synthetic construct
EVQLVQSGAEVKKPGESLKISCKGSGYTFTNYWIHWVRQMPGKGLEWMGYTDPRTDYTEYNQKFKDQVTISADKSISTAYLQWSSLKASDTAMYYCARGGRVGLGYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 81 - protein, 443 aa, Artificial Sequence
  synthetic construct
QVQLVQSGAEVKKPGSSVKVSCKASGYTFTNYWIHWVRQAPGQGLEWMGYTDPRTDYTEYAQKFQGRVTITADESTSTAYMELSSLRSEDTAVYYCARGGRVGLGYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 82 - protein, 444 aa, Artificial Sequence
  synthetic construct
QVQLVQSGAEVKKPGASVQVSCKASGYTFTNYVIHWLRQAPGQGLEWMGYIYPYNDGILYNEKFKGRVTMTSDTSISTAYMELSSLRSDDTAVYYCARGGYYVYDYWGQATLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 83 - protein, 443 aa, Artificial Sequence
  synthetic construct
QVQLVQSGAEVKKPGSSVKVSCKASGYTFTNYWIHWVRQAPGQGLEWMGYTDPRTDYTEYNQKFKDRVTITADESTSTAYMELSSLRSEDTAVYYCARGGRVGLGYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 84 - protein, 444 aa, Artificial Sequence
  synthetic construct
EVQLVQSGAEVKKPGESLKISCKGSGYSFTNYYIHWVRQMPGKGLEWMGYIDPLNGDTTYSPSFQGQVTISADKSISTAYLQWSSLKASDTAMYYCARGGKRAMDYWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 85 - protein, 444 aa, Artificial Sequence
  synthetic construct
EVQLVQSGAEVKKPGESLKISCKGSGYTFTNYVIHWVRQMPGKGLEWMGYIYPYNDGILYNEKFKGQVTISADKSISTAYLQWSSLKASDTAMYYCARGGYYVPDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 86 - protein, 444 aa, Artificial Sequence
  synthetic construct
QVQLVQSGAEVKKPGASVKVSCKASGYTFTNYVIHWVRQAPGQGLEWMGYIYPYNDGILYNEKFKGRVTMTTDTSTSTAYMELRSLRSDDTAVYYCARGGYYVPDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 87 - protein, 443 aa, Artificial Sequence
  synthetic construct
EVQLVQSGAEVKKPGESLKISCKGSGYTFTNYWIHWVRQMPGKGLEWMGYTDPRTDYTEYSPSFQGQVTISADKSISTAYLQWSSLKASDTAMYYCARGGRVGLGYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 88 - protein, 444 aa, Artificial Sequence
  synthetic construct
QVQLVQSGAEVKKPGASVKVSCKASGYSFTNYYIHWVRQAPGQGLEWMGYIDPLNGDTTYNQKFKGRVTMTRDTSTSTVYMELSSLRSEDTAVYYCARGGKRAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 89 - protein, 444 aa, Artificial Sequence
  synthetic construct
QVQLVQSGAEVKKPGSSVKVSCKASGYSFTNYYIHWVRQAPGQGLEWMGYIDPLNGDTTYNQKFKGRVTITADESTSTAYMELSSLRSEDTAVYYCARGGKRAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 90 - protein, 444 aa, Artificial Sequence
  synthetic construct
EVQLVQSGAEVKKPGESLKISCKGSGYSFTNYYIHWVRQMPGKGLEWMGYIDPLNGDTTYNQKFKGQVTISADKSISTAYLQWSSLKASDTAMYYCARGGKRAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 91 - protein, 444 aa, Artificial Sequence
  synthetic construct
QVQLVQSGAEVKKPGSSVKVSCKASGYSFTNYYIHWVRQAPGQGLEWMGYIDPLNGDTTYAQKFQGRVTITADESTSTAYMELSSLRSEDTAVYYCARGGKRAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 92 - protein, 444 aa, Artificial Sequence; identical to SEQ ID NO: 88
  synthetic construct

SEQ ID NO: 93 - protein, 444 aa, Artificial Sequence
  synthetic construct
EVQLQQSGPELMKPGASVKISCKASGYSFTNYYIHWVNQSHGKSLEWIGYIDPLNGDTTYNQKFKGKATLTVDKSSSTAYMRLSSLTSADSAVYYCARGGKRAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 94 - protein, 443 aa, Artificial Sequence
  synthetic construct
QVQLQQFGAELAKPGASVQMSCKASGYTFTNYWIHWVKQRPGQGLEWIGYTDPRTDYTEYNQKFKDKATLAADRSSSTAYMRLSSLTSEDSAVYYCAGGGRVGLGYWGHGSSVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 95 - protein, 443 aa, Artificial Sequence
  synthetic construct
QVQLVQSGAEVKKPGASVKVSCKASGYSFTNYYIHWVRQAPGQGLEWMGYIDPLNGDTTYNQKFKGRVTMTRDTSTSTVYMELSSLRSEDTAVYYCARGGKRAMDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 96 - protein, 443 aa, Artificial Sequence
  synthetic construct
QVQLVQSGAEVKKPGSSVKVSCKASGYSFTNYYIHWVRQAPGQGLEWMGYIDPLNGDTTYNQKFKGRVTITADESTSTAYMELSSLRSEDTAVYYCARGGKRAMDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 97 - protein, 443 aa, Artificial Sequence
  synthetic construct
EVQLVQSGAEVKKPGESLKISCKGSGYSFTNYYIHWVRQMPGKGLEWMGYIDPLNGDTTYNQKFKGQVTISADKSISTAYLQWSSLKASDTAMYYCARGGKRAMDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 98 - protein, 443 aa, Artificial Sequence
  synthetic construct
QVQLVQSGAEVKKPGSSVKVSCKASGYSFTNYYIHWVRQAPGQGLEWMGYIDPLNGDTTYAQKFQGRVTITADESTSTAYMELSSLRSEDTAVYYCARGGKRAMDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 99 - protein, 326 aa, Artificial Sequence
  synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 100 - protein, 214 aa, Artificial Sequence
  synthetic construct
DIQMTQTTSSLSASLGDRVTISCRASQDISNYLNWYQQKPDGTVKLLIYYTSRLYSGVPSRFSGSGSGTDYSLTISNLEQEDIATYFCQQGNTLPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 101 - protein, 219 aa, Artificial Sequence
  synthetic construct
DVLMTQTPLSLPVNLGDQASISCRSRQSIVHTNGNTYLGWFLQKPGQSPKLLIYKVSNRFSGVPDRFSGSGSGTDFTLTISRVEAEDLGVYYCFQGSHVPYTFGGGTKLEIKRADAAPTVSIFPPSMEQLTSGGATVVCFVNNFYPRDISVKWKIDGSEQRDGVLDSVTDQDSKDSTYSMSSTLSLTKVEYERHNLYTCEVVHKTSSSPVVKSFNRNEC

SEQ ID NO: 102 - protein, 446 aa, Artificial Sequence
  synthetic construct
EVQLQQSGPELVKPGASVKMSCKASGYTFTNYVIHWVKRRPGQGLEWIGYIYPYNDGILYNEKFKGKATVTSDKSSSTAYMDLSSLTSEDSAVYYCTRGGYYVPDYWGQGTTLTVSSARTTAPSVYPLVPGCSGTSGSLVTLGCLVKGYFPEPVTVKWNSGALSSGVHTFPAVLQSGLYTLSSSVTVPSSTWSSQTVTCSVAHPATKSNLIKRIEPRRPKPRPPTDICSCDDNLGRPSVFIFPPKPKDILMITLTPKVTCVVVDVSEEEPDVQFSWFVDNVRVFTAQTQPHEEQLNGTFRVVSTLHIQHQDWMSGKEFKCKVNNKDLPSPIEKTISKPRGKARTPQVYTIPPPREQMSKNKVSLTCMVTSFYPASISVEWERNGELEQDYKNTLPVLDSDESYFLYSKLSVDTDSWMRGDIYTCSVVHEALHNHHTQKNLSRSPGK

SEQ ID NO: 103 - protein, 216 aa, Artificial Sequence
  synthetic construct
DVLMTQTPLSLPVNLGDQASISCRSRQSIVHTNGNTYLGWFLQKPGQSPKLLIYKVSNRFSGVPDRFSGSGSGTDFTLTISRVEAEDLGVYYCFQGSHVPYTFGGGTKLEIKRDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 104 - protein, 440 aa, Artificial Sequence
  synthetic construct
EVQLQQSGPELVKPGASVKMSCKASGYTFTNYVIHWVKRRPGQGLEWIGYIYPYNDGILYNEKFKGKATVTSDKSSSTAYMDLSSLTSEDSAVYYCTRGGYYVPDYWGQGTTLTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

(19) United States
(12) Patent Application Publication
(10) Pub. No.: US 2022/0185886 A1
(43) Pub. Date: Jun. 16, 2022

(54) METHODS FOR IDENTIFYING BIOMARKERS TO PREDICT TREATMENT RESPONSE

(71) Applicant: Arch Oncology, Inc., Brisbane, CA (US)

(72) Inventors: Daniel PEREIRA, San Diego, CA (US); Myriam BOUCHLAKA, St. Louis, MO (US); Benjamin J. CAPOCCIA, Webster Groves, MO (US); Casey W. WILSON, St. Louis, MO (US); Robyn PURO, St. Louis, MO (US); Rachel DELSTON, St. Louis, MO (US)

(21) Appl. No.: 17/685,629

(22) Filed: Mar. 3, 2022

Related U.S. Application Data
(63) Continuation of application No. PCT/US2020/049195, filed on Sep. 3, 2020.
(60) Provisional application No. 62/895,327, filed on Sep. 3, 2019.

(51) Int. Cl. C07K 16/28; G01N 33/574; A61P 35/00

(52) U.S. Cl. CPC C07K 16/2803; G01N 33/57426; A61K 2039/505; A61P 35/00; G01N 33/57484

(57) ABSTRACT

Provided are methods for treatment of cancer. Also provided are methods for treating a patient susceptible or suspected of being susceptible with anti-CD47 therapies.

### CROSS REFERENCE TO RELATED APPLICATIONS

This application is a continuation of International Application No. PCT/US2020/049195, filed Sep. 3, 2020, which claims priority to U.S. Provisional Application No. 62/895,327, filed Sep. 3, 2019, each of which is incorporated by reference herein in its entirety for all purposes.

### INCORPORATION BY REFERENCE OF SEQUENCE LISTING

The contents of the text file submitted electronically herewith are incorporated herein by reference in their entirety: A computer readable format copy of the Sequence Listing (filename: ARCO-012_01US_SeqList_ST_25.txt, date recorded: Mar. 1, 2022, file size ˜192 Kilobytes).

### FIELD OF THE DISCLOSURE

The present disclosure includes an evaluation of the sensitivity of patients to medical treatment, for example to identify biomarkers sensitivity to immunotherapy, and a method for treatment using anti-CD47 antibodies.

### BACKGROUND OF THE DISCLOSURE

The development of cancer immunotherapies is occurring at a rapid pace. These immunotherapy treatments enhance the cytotoxic activity of cells of the immune system and have resulted in improved survival of patients with tumor types as diverse as melanoma, non-small cell lung cancer, bladder cancer, and Hodgkin's lymphoma. Despite these positive results, there remains a significant patient population that fail to respond to prescribed immunotherapy treatment or respond initially only to eventually acquire resistance.

The identification and use of biomarkers in the clinic would significantly improve the use of these immunotherapies. Not only would health care providers be able to identify patients that are most likely to benefit from these therapies, biomarkers would avoid treatment-related toxicity and increase our understanding of modes of action of immunotherapy and thereby identify potential combination therapies.

Furthermore, discovering and validating new biomarkers remains an extremely active area of research and development, especially with respect to: other types of immune cells, the complexity of tumor-immune interactions, and the steps involved in the process of the immune system launching an adaptive response against tumors. Thus, there remains a need for advances in biomarker development to further understand the relationship between cancer, the immune system, and the efficacy of immunotherapies.

### BRIEF DESCRIPTION OF THE DRAWINGS

FIG. 1. shows the xenograft model workflow for all four models tested which include implantation, treatment, tumor collection and analysis by NanoString.

### DETAILED DESCRIPTION OF THE DISCLOSURE

The present disclosure provides methods of treatment of cancer in a patient. The methods use multiple assays of biomarkers.

In certain embodiments, the method treats cancer in a patient by measuring at least one biomarker having an expression level greater or less than the amount of a baseline standard by administering an effective amount of an anti-CD47 antibody to the patient, wherein the at least one biomarker is selected from Table 1-Table 20.

In additional embodiments, the method of treats cancer in a patient with an anti-CD47 antibody wherein the method determines expression level of at least one biomarker chosen from the biomarkers listed in Table 1-Table 20; measures the in the level of expression of at least one biomarker indicating sensitivity to treatment, estimates the susceptibility of a patient; and administers a therapeutically effective amount of an anti-CD47 antibody to the patient.

In certain embodiments, the method of quantifying the amount of the biomarker in each sample is not limited to one or more methods selected from NanoString sequencing, RNAseq, qPCR, and microarray.

In certain embodiments, the cancer is a solid tumor.

In certain embodiments, the solid tumor is selected from cervical cancer, pancreatic cancer, ovarian cancer, mesothelioma, squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulval cancer, thyroid cancer, hepatic carcinoma, anal carcinoma, penile carcinoma head and neck cancer, sarcoma, adrenocortical carcinoma, adult (primary) hepatocellular cancer, adult (primary) liver cancer or any combination thereof.

In certain embodiments, the cancer is a hematological malignancy.

In certain embodiments, the hematologic malignancy is multiple myeloma, acute childhood lymphoblastic leukemia, acute lymphoblastic leukemia, acute lymphocytic leukemia, acute myeloid leukemia, adult acute lymphocytic leukemia, adult acute myeloid leukemia, adult Hodgkin's disease, adult Hodgkin's lymphoma, adult lymphocytic leukemia, adult Non-Hodgkin's lymphoma, AIDS-related lymphoma, or any combination thereof.

In certain embodiments, the biological sample is selected from, but not limited to a core biopsy, a free needle aspirate, a pleural effusion, a resection, ascites, whole blood, blood serum, plasma, bone marrow, or other bodily fluid, or dilution thereof.

In certain embodiments, at least two biomarkers are selected from Table 1 through Table 20.

In certain embodiments, at least three biomarkers are selected from Table 1 through Table 20.

In certain embodiments, at least four biomarkers are selected from Table 1 through Table 20.

In certain embodiments, at least five biomarkers are selected from Table 1 through Table 20.

In certain embodiments, greater than five biomarkers are selected from Table 1 through Table 20.

In certain embodiments, the baseline standard is a unit of measurement which provides a calibrated level of the biological effect which may occur prior to the administration of a therapy; i.e. an anti-CD47 antibody. As described herein, you may achieve a baseline standard by measuring gene expression levels for the one or more biomarkers described herein, in untreated tumor cells; i.e., without the administration of an anti-CD47 antibody to establish a baseline standard for treated tumor cells; i.e., with the administration of an anti-CD47 antibody when measured in the same patient.

In certain embodiments, the baseline standard is established for different tumor cell types; i.e., solid tumors and hematological tumors in an untreated patient when measured in the same patient.

In certain embodiments, the anti-CD47 antibodies disclosed block the CD47/SIRPα interaction and the ‘don't eat me’ signal.

In certain embodiments, the anti-CD47 antibodies disclosed induce cell death in solid and hematopoietic cell tumor lines.

In certain embodiments, the anti-CD47 antibodies disclosed have tumor cell binding selectivity compared to normal cells, particularly, binding negligibly to red blood cells (RBCs) in contrast to tumor cells even at high concentrations of antibody.

In certain embodiments, the anti-CD47 antibodies disclosed comprise a combination of a heavy chain (HC) and a light chain (LC), wherein the anti-CD47 antibody is selected from:
(i) a heavy chain comprising the amino acid sequence of SEQ ID NO:76 and a light chain comprising the amino acid sequence SEQ ID NO:66;
(ii) a heavy chain comprising the amino acid sequence of SEQ ID NO:77 and a light chain comprising the amino acid sequence SEQ ID NO:68;
(iii) a heavy chain comprising the amino acid sequence of SEQ ID NO:78 and a light chain comprising the amino acid sequence SEQ ID NO:69;
(iv) a heavy chain comprising the amino acid sequence of SEQ ID NO:79 and a light chain comprising the amino acid sequence SEQ ID NO:70;
(v) a heavy chain comprising the amino acid sequence of SEQ ID NO: 80 and a light chain comprising the amino acid sequence SEQ ID NO:70;
(vi) a heavy chain comprising the amino acid sequence of SEQ ID NO:81 and a light chain comprising the amino acid sequence SEQ ID NO:70;
(vii) a heavy chain comprising the amino acid sequence of SEQ ID NO: 82 and a light chain comprising the amino acid sequence SEQ ID NO:68;
(viii) a heavy chain comprising the amino acid sequence of SEQ ID NO: 83 and a light chain comprising the amino acid sequence SEQ ID NO:70;
(ix) a heavy chain comprising the amino acid sequence of SEQ ID NO: 84 and a light chain comprising the amino acid sequence SEQ ID NO:71;
(x) a heavy chain comprising the amino acid sequence of SEQ ID NO: 85 and a light chain comprising the amino acid sequence SEQ ID NO:72;
(xi) a heavy chain comprising the amino acid sequence of SEQ ID NO: 86 and a light chain comprising the amino acid sequence SEQ ID NO:72;
(xii) a heavy chain comprising the amino acid sequence of SEQ ID NO: 80 and a light chain comprising the amino acid sequence SEQ ID NO:73;
(xiii) a heavy chain comprising the amino acid sequence of SEQ ID NO:81 and a light chain comprising the amino acid sequence SEQ ID NO:73;
(xiv) a heavy chain comprising the amino acid sequence of SEQ ID NO: 87 and a light chain comprising the amino acid sequence SEQ ID NO:70;
(xv) a heavy chain comprising the amino acid sequence of SEQ ID NO:79 and a light chain comprising the amino acid sequence SEQ ID NO:73;
(xvi) a heavy chain comprising the amino acid sequence of SEQ ID NO: 88 and a light chain comprising the amino acid sequence SEQ ID NO:74;
(xvii) a heavy chain comprising the amino acid sequence of SEQ ID NO: 89 and a light chain comprising the amino acid sequence SEQ ID NO:74;
(xviii) a heavy chain comprising the amino acid sequence of SEQ ID NO:90 and a light chain comprising the amino acid sequence SEQ ID NO:74;
(xix) a heavy chain comprising the amino acid sequence of SEQ ID NO:91 and a light chain comprising the amino acid sequence SEQ ID NO:74;
(xx) a heavy chain comprising the amino acid sequence of SEQ ID NO: 84 and a light chain comprising the amino acid sequence SEQ ID NO:74;
(xxi) a heavy chain comprising the amino acid sequence of SEQ ID NO:92 and a light chain comprising the amino acid sequence SEQ ID NO:71;
(xxii) a heavy chain comprising the amino acid sequence of SEQ ID NO: 89 and a light chain comprising the amino acid sequence SEQ ID NO:71;
(xxiii) a heavy chain comprising the amino acid sequence of SEQ ID NO:90 and a light chain comprising the amino acid sequence SEQ ID NO:31;
(xxiv) a heavy chain comprising the amino acid sequence of SEQ ID NO:91 and a light chain comprising the amino acid sequence SEQ ID NO:71;
(xxv) a heavy chain comprising the amino acid sequence of SEQ ID NO: 85 and a light chain comprising the amino acid sequence SEQ ID NO:68;
(xxvi) a heavy chain comprising the amino acid sequence of SEQ ID NO: 86 and a light chain comprising the amino acid sequence SEQ ID NO:68;
(xxvii) a heavy chain comprising the amino acid sequence of SEQ ID NO:93 and a light chain comprising the amino acid sequence SEQ ID NO:100;
(xxviii) a heavy chain comprising the amino acid sequence of SEQ ID NO:94 and a light chain comprising the amino acid sequence SEQ ID NO:75;
(xxix) a heavy chain comprising the amino acid sequence of SEQ ID NO:95 and a light chain comprising the amino acid sequence SEQ ID NO:71;
(xxx) a heavy chain comprising the amino acid sequence of SEQ ID NO:96 and a light chain comprising the amino acid sequence SEQ ID NO:71;
(xxxi) a heavy chain comprising the amino acid sequence of SEQ ID NO:97 and a light chain comprising the amino acid sequence SEQ ID NO:71;
(xxxii) a heavy chain comprising the amino acid sequence of SEQ ID NO:98 and a light chain comprising the amino acid sequence SEQ ID NO:71;
(xxxiii) a heavy chain comprising the amino acid sequence of SEQ ID NO:83 and a light chain comprising the amino acid sequence SEQ ID NO:73;
(xxxiv) a heavy chain comprising the amino acid sequence of SEQ ID NO:87 and a light chain comprising the amino acid sequence SEQ ID NO:73;
(xxxv) a heavy chain comprising the amino acid sequence of SEQ ID NO:102 and a light chain comprising the amino acid sequence SEQ ID NO:101;
(xxxvi) a heavy chain comprising the amino acid sequence of SEQ ID NO:104 and a light chain comprising the amino acid sequence SEQ ID NO:103; wherein the VH amino acid sequence is at least 90%, 95%, 97%, 98% or 99% identical thereto and the VL amino acid sequence is at least 90%, 95%, 97%, 98% or 99% identical thereto.

### Checkpoints and Checkpoint Inhibition

Therapeutic antibodies targeting the T cell checkpoints, PD-1, PD-L1 and CTLA-4 to enhance the cytotoxic activity of the adaptive T cell immune response have raised the prospect of long-term remission or even cure for patients with metastatic diseases [Hodi, 2010, McDermott, 2015]. Despite positive results, there remains a significant patient population that either fails to respond to these checkpoint inhibitors (primary resistance) or those that respond, but eventually develop disease progression (acquired resistance) [Pitt 2016, Restifo 2016, Sharma 2017]. Recent studies suggest that resistance mechanisms can be both tumor cell intrinsic, including a lack of unique tumor antigen proteins or inhibition of tumor antigen presentation, and tumor cell extrinsic, involving the absence of infiltrating T cells, redundant inhibitory checkpoints and/or the presence of immunosuppressive cells in the tumor microenvironment [Sharma 2017]. Even in tumors considered sensitive to checkpoint inhibitors, or when combining anti-CTLA-4 and anti-PD-1/PDL-1 agents, approximately 50% of patients do not experience tumor shrinkage and the median treatment duration or progression-free survival for all treated patients remains relatively short around 2-5 months [Kazandjian 2016]. In addition, several of the most prevalent solid tumors and the majority of hematological malignancies have shown disappointing results with these checkpoint inhibitors. In particular, hormone receptor-positive breast cancer, colorectal cancer (non-microsatellite instability) and prostate cancer do not appear to be sensitive to this type of immune manipulation and could benefit from a different immunotherapy approach [Le 2015, Dirix 2015, Topalian 2012, Graff 2016]. These findings highlight the need for alternative or synergistic approaches that target additional checkpoints to activate the innate immune response in addition to the adaptive immune response to further improve clinical outcomes.

CD47 Biology and Role as Innate Immune Checkpoint

CD47, also known as integrin associated protein (IAP), is a 50 kDa cell surface Ig superfamily member containing an extracellular IgV domain, 5 transmembrane domains, and a short C-terminal cytoplasmic tail, that is expressed on most cells and overexpressed on many cancer subtypes [Lindberg 1993, Reinhold 1995, Majetti 2009, Willingham 2012]. The functional activities of CD47 are defined by its two distinct ligands, signal regulatory protein alpha (SIRPα) and thrombospondin 1 (TSP1) [Gao 1994, Barclay 2006]. TSP is present on plasma and is synthesized by many cells, including platelets. SIRPα is expressed on many hematopoietic cells, including macrophages, dendritic cells, granulocytes and on a number of other cell types including neurons and glia. The CD47/SIRPα interaction functions as a marker of self, regulating macrophage and dendritic cell phagocytosis of target cells sending a “don't eat me signal” to the phagocyte. The binding of CD47 to SIRPα initiates an inhibitory signaling cascade resulting in inhibition of phagocytosis following phosphorylation of its cytoplasmic immunoreceptor tyrosine-based inhibition motifs (ITIMs) [Oldenborg, 2000, Oldenborg 2001, Okazawa, 2005], recruitment and binding of SHP1 and SHP2, Src homology domain-containing protein tyrosine phosphatases [Veillette, 1998, Oldenborg, 2001], inhibition of non-muscle myosin IIA and ultimately phagocytic function [Tsai and Discher, 2008, Barclay and van den Berg, 2014, Murata, 2014, Veillette and Chen, 2018, Matazaki, 2009].

Several anti-CD47 mouse antibodies have been described that block the interaction of CD47 and SIRPα, including B6H12 [Seiffert 1999, Latour 2001, Subramanian 2006, Liu 2002, Rebres 2005], BRIC126 [Vernon-Wilson 2000, Subramanian 2006], CC2C6 [Seiffert 1999] and 1F7 [Rebres 2005]. B6H12 and BRIC126 have also been shown to cause phagocytosis of human tumor cells by human and mouse macrophages [Willingham 2012, Chao 2012, EP 2 242 512 B1]. This increase in phagocytic activity resulting from blocking the CD47/SIRPα is the primary mechanism of action for the CD47 antibodies currently in early clinical studies [Barclay 2014, Weiskopf 2017]. This mechanism serves as one of the most important checkpoints regulating innate immune activation. Anti-CD47 mAbs have also been shown to promote an adaptive immune response to tumors in vivo [Tseng 2013, Soto-Pantoja 2014, Liu 2015]. Cancer cells thus use CD47 to mask themselves in “selfness” to evade both the innate and adaptive immune systems.

### Induction of Programmed Cell Death III (PCDIII)

A number of anti-CD47 antibodies (CD47 mAbs), including Ad22, 1F7, MABL-1, MABL-2, and CC2C6, indicated that some, but not all, soluble CD47 mAbs, as well as some additional immobilized CD47 mAbs, can directly elicit a type of cell death of multiple types of tumor cells that is characteristic programmed cell death III (PCDIII) [Lie 1999, Manna 2003, Manna 2004, Mateo 1999, Mateo 2002, Uno 2005, Bras 2007, Martinez-Tones, 2015, Leclair, 2018]. PCDIII is caspase-independent and includes cellular features such as production of reactive oxygen species (ROS), loss of mitochondrial membrane potential (Δψₘ) and exposure of phosphatidylserine (PS) on the plasma membrane without the interaction with any immune effector cell and without nuclear features including chromatin condensation, DNA fragmentation and degradation [Kikuchi 2005, Pettersen 1999, Manna 2003, Manna 2004, Sagawa 2011, Uno 2007, Mateo 1999, Mateo 2002, Roue, 2003]. It is noteworthy that the anti-CD47 killing antibodies do not kill resting leukocytes, which also express CD47, but only those cells that are “activated” by transformation. Thus, normal circulating cells, many of which express CD47, are spared while cancer cells are selectively killed by the CD47 antibodies that possess this direct killing activity [Manna and Frazier 2003].

### Induction of Immunogenic Cell Death (ICD)

The concept of immunogenic cell death (ICD) has emerged in recent years. This form of cell death, unlike non-immunogenic cell death, stimulates an adaptive immune response against tumor antigens presented to T cells [Casares 2005, Krysko 2012, Kroemer 2012]. ICD is induced by specific chemotherapy drugs, including anthracyclines (doxorubicin, daurorubicin and mitoxantrone) and oxaliplatin, but not by cisplatin and other chemotherapy drugs. ICD is also induced by bortezomib, cardiac glycosides, photodynamic therapy and radiation [Galluzi 2016]. ICD is characterized by the release or surface exposure of damage-associated molecular patterns (DAMPs) from dying cells that function as adjuvants for the immune system [Kroemer 2013]. The distinctive characteristics of ICD of tumor cells are the release from or exposure on tumor cell surfaces these DAMPs including: 1) the pre-apoptotic cell surface exposure of calreticulin, 2) the secretion of adenosine triphosphate (ATP), 3) release of high mobility group box 1 (HMGB1), 4) annexin Al release, 5) type I interferon release and 6) C-X-C motif chemokine ligand 10 (CXCL10) release. These ligands are endogenous damage-associated molecular patterns (DAMPs), which include the cell death-associated molecules (CDAMs). Importantly, each of these ligands induced during ICD binds to specific receptors, referred to as pattern recognition receptors (PRRs), that contribute to an anti-tumor immune response. ATP binds the purinergic receptors PY2, G-protein coupled, 2 (P2RY2) and PX2, ligand-gated ion channel, 7 (P2RX7) on dendritic cells causing dendritic cell recruitment and activation, respectively. Annexin A1 binds to formyl peptide receptor 1 (FPR1) on dendritic cells causing dendritic cell homing. Calreticulin expressed on the surface of tumor cells binds to LRP1 (CD91) on dendritic cells promoting antigen uptake by dendritic cells. HMGB1 binds to toll-like receptor 4 (TLR4) on dendritic cells to cause dendritic cell maturation. As a component of ICD, tumor cells release type I interferon leading to signaling via the type I interferon receptor and the release of the CXCL10 which favors the recruitment of effector CXCR3+ T cells Together, the actions of these ligands on their receptors facilitate recruitment of DCs into the tumor, the engulfment of tumor antigens by DCs and optimal antigen presentation to T cells. Kroemer et al. have proposed that a precise combination of the CDAMs mentioned above elicited by ICD can overcome the mechanisms that normally prevent the activation of anti-tumor immune responses [Kroemer 2016]. When mouse tumor cells treated in vitro with ICD-inducing modalities are administered in vivo to syngeneic mice, they provide effective vaccination that leads to an anti-tumor adaptive immune response, including memory. This vaccination effect cannot be tested in xenograft tumor models because the mice used in these studies lack a complete immune system. The available data indicate that ICD effects induced by chemotherapy or radiation will promote an adaptive anti-tumor immune response in cancer patients. The components of ICD are described in more detail below.

In 2005, it was reported that tumor cells which were dying in response to anthracycline chemotherapy in vitro caused an effective anti-tumor immune response when administered in vivo in the absence of adjuvant [Casares 2005]. This immune response protected mice from subsequent re-challenge with viable cells of the same tumor and caused regression of established tumors. Anthracyclines (doxorubicin, daunorubicin and idarubicin) and mitomycin C induced tumor cell apoptosis with caspase activation, but only apoptosis induced by anthracyclines resulted in immunogenic cell death. Caspase inhibition did not inhibit cell death induced by doxorubicin but did suppress the immunogenicity of tumor cells dying in response to doxorubicin. The central roles of dendritic cells and CD8+ T cells in the immune response elicited by doxorubicin-treated apoptotic tumor cells was established by the demonstration that depletion of these cells abolished the immune response in vivo.

Calreticulin is one of the most abundant proteins in the endoplasmic reticulum (ER). Calreticulin was shown to rapidly translocate pre-apoptotically from the ER lumen to the surface of cancer cells in response to multiple ICD inducers, including anthracyclines [Obeid 2007, Kroemer 2013]. Blockade or knockdown of calreticulin suppressed the phagocytosis of anthracycline-treated tumor cells by dendritic cells and abolished their immunogenicity in mice. The exposure of calreticulin caused by anthracyclines or oxaliplatin is activated by an ER stress response that involves the phosphorylation of the eukaryotic translation initiation factor eIF2α by the PKR-like ER kinase. Calreticulin, which has a prominent function as an “eat-me” signal [Gardai 2005] binds to LRP1 (CD91) on dendritic cells and macrophages resulting in phagocytosis of the calreticulin expressing cell, unless the calreticulin-expressing cell expresses a don't eat me signal, such as CD47. Calreticulin also signals through CD91 on antigen presenting cells to cause the release of proinflammatory cytokines and to program Th17 cell responses. In summary, calreticulin expressed as part of ICD stimulates antigen presenting cells to engulf dying cells, process their antigens and prime an immune response.

In addition to calreticulin, protein disulfide-isomerase A3 (PDIA3), also called Erp57, was shown to translocate from the ER to the surface of tumor cells following treatment with mitoxantrone, oxaliplatin and irradiation with UVC light [Panaretakis 2008, Panaretakis 2009]. A human ovarian cancer cell line, primary ovarian cancer cells and a human prostate cancer cell line expressed cell-surface calreticulin, HSP70 and HSP90 following treatment with the anthracyclines doxorubicin and idarrubicin [Fucikova 2011]. HSP70 and HSP90 bind to the PRR LRP1 on antigen presenting cells; the PRR to which PDIA3 binds has not been identified [Galluzi 2016].

TLR4 was shown to be required for cross-presentation of dying tumor cells and to control tumor antigen processing and presentation. Among proteins that were known to bind to and stimulate TLR4, HMGB1 was uniquely released by mouse tumor cells in which ICD was induced by irradiation or doxorubicin [Apetoh 2007]. The highly efficient induction of an in vivo anti-tumor immune by doxorubicin treatment of mouse tumor cells required the presence of HMGB1 and TLR4, as demonstrated by abrogation of the immune response by inhibition of HMGB1 and knock-out TLR4. These preclinical findings are clinically relevant. Patients with breast cancer who carry a TLR4 loss-of-function allele relapse more quickly after radiotherapy and chemotherapy than those carrying the normal TLR4 allele.

Ghiringhelli et al. showed that mouse tumor cells treated with oxaliplatin, doxorubicin and mitoxantrone in vitro released ATP and that the ATP binds to the purinergic receptors PY2, G-protein coupled, 2 (P2RY2) and PX2, ligand-gated ion channel, 7 (P2RX7) on dendritic cells [Ghiringhelli 2009]. Binding of ATP to P2RX7 on DCs triggers the NOD-like receptor family, pyrin domain containing-3 protein (NLRP3)-dependent caspase-1 activation complex (inflammasome), allowing for the secretion of interleukin-β (IL-1β), which is essential for the priming of interferon-gamma-producing CD8+ T cells by dying tumor cells. Therefore, the ATP-elicited production of IL-1β by DCs appears to be one of the critical factors for the immune system to perceive cell death induced by certain chemotherapy drugs as immunogenic. This paper also reports that HMGB1, at TLR4 agonist, also contributes to the stimulation of the NLRP3 inflammasome in DCs and the secretion of IL-1β. These preclinical results have been shown to have clinical relevance; in a breast cancer cohort, the presence of the P2RX7 loss-of-function allele had a significant negative prognostic impact of metastatic disease-free survival. ATP binding to P2RY2 causes the recruitment of myeloid cells into the tumor microenvironment [Vacchelli 2016].

Michaud et al. demonstrated that autophagy is required for the immunogenicity of chemotherapy-induced cell death [Michaud 2011]. Release of ATP from dying tumor cells required autophagy and autophagy-competent, but not autophagy-deficient, mouse tumors attracted dendritic cells and T lymphocytes into the tumor microenvironment in response to chemotherapy that induces ICD.

Ma et al. addressed the question of how chemotherapy-induced cell death leads to efficient antigen presentation to T cells [Ma 2013]. They found that at specific kind of tumor infiltrating lymphocyte, CD11c+CD11b+Ly6Chi cells, are particularly important for the induction of anticancer immune responses by anthracyclines. ATP released by dying cancer cells recruited myeloid cells into tumors and stimulated the local differentiation of CD11c⁺CD11b⁺Ly6Chi cells. These cells were shown to be particularly efficient in capturing and presenting tumor cell antigens and, after adoptive transfer into naive mice, conferring protection to challenge with living tumor cells of the same cell line.

It has been shown that anthracyclines stimulate the rapid production of type I interferons by tumor cells after activation of TLR3 [Sistugu 2014]. Type I interferons bind to IFNγ and IFNγ receptors on cancer cells and trigger autocrine and paracrine signaling pathways that result in release of CXCL10. Tumors lacking Tlr3 or Ifnar failed to respond to chemotherapy unless type I IFN or CXCL10, respectively, was supplied. These preclinical findings have clinical relevance. A type I IFN-related gene expression signature predicted clinical responses to anthracycline-based chemotherapy in independent cohorts of breast cancer patients.

Another receptor on dendritic cells that is involved in chemotherapy-induced anti-cancer immune response was recently identified: formyl peptide receptor-1, which binds annexin A1 [Vacchelli 2015]. Vacchelli et al designed a screen to identify candidate genetic defects that negatively affect responses to chemotherapy. They identified a loss-of-function allele of the gene encoding formyl peptide receptor 1 (FPR1) that was associated with poor metastatis-free survival and overall survival in breast and colorectal cancer patients receiving adjuvant chemotherapy. The therapeutic effects of anthracyclines were abrogated in tumor-bearing Fpr1−/− mice due to impaired antitumor immunity. FPR1-deficient DCs did not approach dying tumor cells and, therefore, could not elicit antitumor T cell immunity. Two anthracyclines, doxorubicin and mitoxantrone, stimulated the secretion of annexin A1, one of four known ligands of FPR1. FPR1 and annexin A1 promoted stable interactions between dying cancer cells and human or mouse leukocytes.

In addition to anthracyclines and oxaliplatin, other drugs have been shown to induce immunogenic cell death. Cardiac glycosides, including clinically used digoxin and digitoxin, were also shown to be efficient inducers of immunogenic cell death of tumor cells [Menger 2012]. Other chemotherapy agents and cancer drugs that have been reported to induce DAMP expression or release are bleomycin, bortezomib, cyclophosphamide, paclitaxel, vorinistat and cisplatin [Garg 2015, Menger 2012, Martins 2011]. Importantly, these results have clinical relevance. Administration of digoxin during chemotherapy had a significant positive impact on the overall survival of patients with breast, colorectal, head and neck, and hepatocellular cancers, but failed to improve overall survival of lung and prostate cancer patients.

The anti-CD20 monoclonal antibody rituximab has improved outcomes in multiple B-cell malignancies. The success of rituximab, referred to as a type I anti-CD20 mAb, led to the development of type II anti-CD20 mAbs, including obinutuzumab and tositumomab. Cheadle et al investigated the induction of immunogenic cell death by anti-CD20 mAbs [Cheadle 2013]. They found that the cell death induced by obinutuzumab and tositumomab is a form of immunogenic cell death characterized by the release of HMGB1, HSP90 and ATP. A type I anti-CD20 mAb did not cause release of HMGB1, HSP90 and ATP. Incubation of supernatants from a human tumor cell line treated with obinutuzumab caused maturation of human dendritic cells, consistent with the previously described effects of HMGB1 and ATP on dendritic cells. In contrast to the results reported by Cheadle et al, Zhao et al reported that both type I and II anti-CD20 mAbs increased HMGB1 release from human diffuse large B cell lymphoma cell lines, but did not cause ATP release or cell surface expression of calreticulin [Zhao 2015].

### Definitions

As used herein, the term “patient” as used herein refers to a human, for whom a classification as a responder to a next generation immune checkpoint inhibitor is desired, and for whom further treatment can be provided.

As used herein, the term “the baseline standard” is a unit of measurement which allows for calibration of the biological effects which may occur after the administration of a therapy; i.e. a killing anti-CD47 antibody.

As used herein, the term “biomarker” is a biological molecule found in blood, other body fluids, or tissues that is a sign of a normal or abnormal process, a condition, or disease. A biomarker may be used to see how well the body responds to a treatment for a disease; e.g., administration of anti-CD47 therapy. The term “biomarker” can be used interchangeably with molecular marker and/or signature molecule.

As used herein, the term, “NanoString” refers to a robust and highly reproducible method for detecting the expression of up to 800 genes in a single reaction with high sensitivity and linearity across a broad range of expression levels. The methodology serves to bridge the gap between genome-wide (microarrays) and targeted (real-time quantitative PCR) expression profiling.

As used herein, the terms “tumor” or “tumor tissue” refer to an abnormal mass of tissue that results from excessive cell division. A tumor or tumor tissue comprises “tumor cells” which are neoplastic cells with abnormal growth properties and no useful bodily function. Tumors, tumor tissue and tumor cells may be benign or malignant. A tumor or tumor tissue may also comprise “tumor-associated non-tumor cells”, e.g., vascular cells which form blood vessels to supply the tumor or tumor tissue. Non-tumor cells may be induced to replicate and develop by tumor cells, for example, the induction of angiogenesis in a tumor or tumor tissue.

As used herein, the term “malignancy” refers to a non-benign tumor or a cancer.

As used herein, the term “cancer” connotes a type of hyperproliferative disease which includes a malignancy characterized by deregulated or uncontrolled cell growth.

The following examples are included to demonstrate preferred embodiments of the disclosure. The following examples are presented only by way of illustration and to assist one of ordinary skill in using the disclosure. The examples are not intended in any way to otherwise limit the scope of the disclosure. Those of skill in the art should, in light of the present disclosure, appreciate that many changes can be made in the specific embodiments which are disclosed and still obtain a like or similar result without departing from the spirit and scope of the disclosure.

### EXAMPLES

### Example 1

Amino Acid Sequences

| LCDR1 | LCDR2 | LCDR3 |
|---|---|---|
| Light Chain CDRs | | |
| Vx4-LCDR1 | Vx4-LCDR2 | Vx4-LCDR3 |
| RSRQSIVHTNGNTYLG | KVSNRFS | FQGSHVPYT |
| (SEQ ID NO: 11) | (SEQ ID NO: 15) | (SEQ ID NO: 18) |
| Vx8-LCDR1 | Vx8-LCDR2 | Vx8-LCDR3 |
| RASQDISNYLN | YTSRLYS | QQGNTLPWT |
| (SEQ ID NO: 12) | (SEQ ID NO: 16) | (SEQ ID NO: 19) |
| Vx8-LCDR1 | | |
| RASQSISNYLN | | |
| (SEQ ID NO: 13) | | |
| Vx9-LCDR1 | Vx9-LCDR2 | Vx9-LCDR3 |
| RSSQNIVQSNGNTYLE | KVFHRFS | FQGSHVPWT |
| (SEQ ID NO: 14) | (SEQ ID NO: 17) | (SEQ ID NO: 20) |
| Heavy Chain CDRs | | |
| HCDR1 | HCDR2 | HCDR3 |
| Vx4-HCDR1 | Vx4-HCDR2 | Vx4-HCDR3 |
| GYTFTNYVIH | YIYPYNDGILYNEKFKG | GGYYVPDY |
| (SEQ ID NO: 1) | (SEQ ID NO: 4) | (SEQ ID NO: 7) |
| | | Vx4-HCDR3 |
| | | GGYYVYDY |
| | | (SEQ ID NO: 8) |
| Vx8-HCDR1 | Vx8-HCDR2 | Vx8-HCDR3 |
| GYSFTNYYIH | YIDPLNGDTTYNQKFKG | GGKRAMDY |
| (SEQ ID NO: 2) | (SEQ ID NO: 5) | (SEQ ID NO: 9) |
| Vx9-HCDR1 | Vx9-HCDR2 | Vx9-HCDR3 |
| GYTFTNYWIH | YTDPRTDYTEYNQKFKD | GGRVGLGY |
| (SEQ ID NO: 3) | (SEQ ID NO: 6) | (SEQ ID NO: 10) |

Murine Light Chain Variable Domains
>Vx4murL01
DVLMTQTPLSLPVNLGDQASISCRSRQSIVHTNGNTYLGWFLQKPGQSPKLLIYKVS
NRFSGVPDRFSGSGSGTDFTLTISRVEAEDLGVYYCFQGSHVPYTFGGGTKLEIK (SEQ
ID NO: 41).
>Vx4murL02
DVLMTQTPLSLPVNLGDQASISCRSRQSIVHTNGNTYLGWFLQKPGQSPKLLIYKVS
NRFSGVPDRFSGSGSGTDFTLTISRVEAEDLGVYYCFQGSHVPYTFGQGTKVEIK
(SEQ ID NO: 42).
>Vx8murL03
DIQMTQTTSSLSASLGDRVTISCRASQDISNYLNWYQQKPDGTVKLLIYYTSRLYSGV
PSRFSGSGSGTDYSLTISNLEQEDIATYFCQQGNTLPWTFGGGTKLEIK (SEQ ID
NO: 46).
>Vx9murL04
DVFMTQTPLSLPVSLGDQASISCRSSQNIVQSNGNTYLEWYLQKPGQSPKLLIYKVFH
RFSGVPDRFSGSGSGTDFTLKISRVEAEDLGVYYCFQGSHVPWTFGGGTKVEIK (SEQ
ID NO: 50)
Murine Heavy Chain Variable Domains
>Vx4murH01
EVQLQQSGPELVKPGASVKMSCKASGYTFTNYVIHWVKRRPGQGLEWIGYIYPYND
GILYNEKFKGKATVTSDKSSSTAYMDLSSLTSEDSAVYYCTRGGYYVPDYWGQGTTL
TVSS (SEQ ID NO: 21).
>Vx4mur-H02
EVQLQQSGPELVKPGASVKMSCKASGYTFTNYVIHWVKRRPGQGLEWIGYIYPYND
GILYNEKFKGKATVTSDKSSSTAYMDLSSLTSEDSAVYYCTRGGYYVPDYVVGQGTL
VTVSS (SEQ ID NO: 22).
>Vx8murH03
EVQLQQSGPELMKPGASVKISCKASGYSFTNYYIHWVNQSHGKSLEWIGYIDPLNGD
TTYNQKFKGKATLTVDKSSSTAYMRLSSLTSADSAVYYCARGGKRAMDYWGQGTSV
TVSS (SEQ ID NO: 28).
>Vx9murH04
QVQLQQFGAELAKPGASVQMSCKASGYTFTNYWIHWVKQRPGQGLEWIGYTDPRT
DYTEYNQKFKDKATLAADRSSSTAYMRLSSLTSEDSAVYYCAGGGRVGLGYWGHGS
SVTVSS (SEQ ID NO: 35)
Human Light Chain Variable Domains
>Vx4humL01
DIVMTQSPLSLPVTPGEPASISCRSRQSIVHTNGNTYLGWYQKPGQSPRLLIYKVSNR
FSGVPDRFSGSGSGTDFTLKISRVEADDVGIYYCFQGSHVPYTFGQGTKLEIK (SEQ ID
NO: 43)
>Vx4humL02
DVVMTQSPLSLPVTLGQPASISCRSRQSIVHTNGNTYLGWFQQRPGQSPRRLIYKVSN
RFSGVPDRFSGSGSGTDFTLKISRVEAEDVGVYYCFQGSHVPYTFGQGTKLEIK (SEQ
ID NO: 44)
>Vx4humL03
DIVMTQSPDSLAVSLGERATINCRSRQSIVHTNGNTYLGWYQQKPGQPPKLLIYKVSN
RFSGVPDRFSGSGSGTDFTLTISSLQAEDVAVYYCFQGSHVPYTFGQGTKLEIK (SEQ
ID NO: 45)
>Vx8humL04
DIQMTQSPSSLSASVGDRVTITCRASQDISNYLNWYQQKPGKAPKLLIYYTSRLYSGV
PSRFSGSGSGTDFTFTISSLQPEDIATYYCQQGNTLPWTFGQGTKVEIK (SEQ ID
NO: 47).
>Vx8humL05
DIQMTQSPSSLSASVGDRVTITCRASQSISNYLNWYQQKPGKAPKLLIYYTSRLYSGV
PSRFSGSGSGTDFTLTISSLQPEDFATYYCQQGNTLPWTFGQGTKVEIK (SEQ ID
NO: 48).
>Vx8humL06
DIVMTQSPLSLPVTPGEPASISCRASQDISNYLNWYLQKPGQSPRLLIYYTSRLYSGVP
DRFSGSGSGTDFTLKISRVEADDVGIYYCQQGNTLPWTFGQGTKLEIK (SEQ ID
NO: 49)
>Vx9humL07
DVVMTQSPLSLPVTLGQPASISCRSSQNIVQSNGNTYLEWFQQRPGQSPRRLIYKVFH
RFSGVPDRFSGSGSGTDFTLKISRVEAEDVGVYYCFQGSHVPYTFGQGTKLEIK (SEQ
ID NO: 51).
>Vx9humL08
DIVMTQSPDSLAVSLGERATINCRSSQNIVQSNGNTYLEWYQQKPGQPPKLLIYKVFH
RFSGVPDRFSGSGSGTDFTLTISSLQAEDVAVYYCFQGSHVPYTFGQGTKLEIK (SEQ
ID NO: 52).
Human Heavy Chain Variable Domains
>Vx4humH01
QVQLVQSGAEVKKPGASVQVSCKASGYTFTNYVIHWLRQAPGQGLEWMGYIYPYN
DGILYNEKFKGRVTMTSDTSISTAYMELSSLRSDDTAVYYCARGGYYVPDYWGQATL
VTVSS (SEQ ID NO: 23).
>Vx4humH02
QVQLVQSGAEVKKPGASVQVSCKASGYTFTNYVIHWLRQAPGQGLEWMGYIYPYN
DGILYNEKFKGRVTMTSDTSISTAYMELSSLRSDDTAVYYCARGGYYVYDYWGQATL
VTVSS (SEQ ID NO: 24).
>Vx4humH03
EVQLVQSGAEVKKPGATVKISCKVSGYTFTNYVIHWVQQAPGKGLEWMGYIYPYND
GILYNEKFKGRVTITADTSTDTAYMELSSLRSEDTAVYYCATGGYYVPDYWGQGTTV
TVSS (SEQ ID NO: 25)
>Vx4humH04
EVQLVQSGAEVKKPGESLKISCKGSGYTFTNYVIHWVRQMPGKGLEWMGYIYPYND
GILYNEKFKGQVTISADKSISTAYLQWSSLKASDTAMYYCARGGYYVPDYWGQGTT
VTVSS (SEQ ID NO: 26)
>Vx4humH05
QVQLVQSGAEVKKPGASVKVSCKASGYTFTNYVIHWVRQAPGQGLEWMGYIYPYN
DGILYNEKFKGRVTMTTDTSTSTAYMELRSLRSDDTAVYYCARGGYYVPDYWGQGT
TVTVSS (SEQ ID NO: 27)
>Vx8humH06
QVQLVQSGAEVKKPGASVKVSCKASGYSFTNYYIHWVRQAPGQGLEWMGYIDPLN
GDTTYNQKFKGRVTMTRDTSTSTVYMELSSLRSEDTAVYYCARGGKRAMDYWGQG
TLVTVSS (SEQ ID NO: 29).
>Vx8humH07
QVQLVQSGAEVKKPGSSVKVSCKASGYSFTNYYIHWVRQAPGQGLEWMGYIDPLN
GDTTYNQKFKGRVTITADESTSTAYMELSSLRSEDTAVYYCARGGKRAMDYWGQGT
LVTVSS (SEQ ID NO: 30).
>Vx8humH08
EVQLVQSGAEVKKPGESLKISCKGSGYSFTNYYIHVVVRQMPGKGLEWMGYIDPLNG
DTTYNQKFKGQVTISADKSISTAYLQWSSLKASDTAMYYCARGGKRAMDYWGQGT
LVTVSS (SEQ ID NO: 31).
>Vx8humH09
QVQLVQSGAEVKKPGSSVKVSCKASGYSFTNYYIHWVRQAPGQGLEWMGYIDPLN
GDTTYAQKFQGRVTITADESTSTAYMELSSLRSEDTAVYYCARGGKRAMDYWGQGT
LVTVSS (SEQ ID NO: 32).
>Vx8humH10
EVQLVQSGAEVKKPGESLKISCKGSGYSFTNYYIHVVVRQMPGKGLEWMGYIDPLNG
DTTYSPSFQGQVTISADKSISTAYLQWSSLKASDTAMYYCARGGKRAMDYWGRGTL
VTVSS (SEQ ID NO: 33).
>Vx8humH11
QVQLVQSGAEVKKPGASVQVSCKASGYSFTNYYIHWLRQAPGQGLEWMGYIDPLN
GDTTYNQKFKGRVTMTSDTSISTAYMELSSLRSDDTAVYYCARGGKRAMDYWGQAT
LVTVSS (SEQ ID NO: 34)
>Vx9humH12
QVQLVQSGAEVKKPGASVKVSCKASGYTFTNYWIHWVRQAPGQGLEWMGYTDPR
TDYTEYNQKFKDRVTMTRDTSTSTVYMELSSLRSEDTAVYYCARGGRVGLGYWGQ
GTLVTVSS (SEQ ID NO: 36).
>Vx9humH13
QVQLVQSGAEVKKPGSSVKVSCKASGYTFTNYVVIHWVRQAPGQGLEWMGYTDPRT
DYTEYNQKFKDRVTITADESTSTAYMELSSLRSEDTAVYYCARGGRVGLGYWGQGTL
VTVSS (SEQ ID NO: 37).
>Vx9humH14
EVQLVQSGAEVKKPGESLKISCKGSGYTFTNYWIHVVVRQMPGKGLEWMGYTDPRT
DYTEYNQKFKDQVTISADKSISTAYLQWSSLKASDTAMYYCARGGRVGLGYWGQG
TLVTVSS (SEQ ID NO: 38).
>Vx9humH15
QVQLVQSGAEVKKPGSSVKVSCKASGYTFTNYVVIHWVRQAPGQGLEWMGYTDPRT
DYTEYAQKFQGRVTITADESTSTAYMELSSLRSEDTAVYYCARGGRVGLGYWGQGTL
VTVSS (SEQ ID NO: 39).
>Vx9humH16
EVQLVQSGAEVKKPGESLKISCKGSGYTFTNYWIHWVRQMPGKGLEWMGYTDPRT
DYTEYSPSFQGQVTISADKSISTAYLQWSSLKASDTAMYYCARGGRVGLGYWGQGT
LVTVSS (SEQ ID NO: 40).
Human IgG-Fc
>Human Fc IgG1
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPE
LLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSF
FLYSKLTVDKSRWQQGNVFSCSVMHEALHNHYTQKSLSLSPGK (SEQ ID NO: 53).
>Human Fc IgG1-N297Q
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPE
LLGGPSVFLFPPKPKDTLMISRTPEVTCVVVDVSHEDPEVKFNWYVDGVEVHNAKT
KPREEQYQSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSF
FLYSKLTVDKSRWQQGNVFSCSVMHEALHNHYTQKSLSLSPGK (SEQ ID NO: 54).
>Human Fc-IgG2
ASTKGPSVFPLAPCSRSTSESTAALGCLVKDYFPEPVTVSWNSGALTSGVHTFPAVLQ
SSGLYSLSSVVTVPSSNFGTQTYTCNVDHKPSNTKVDKTVERKCCVECPPCPAPPVA
GPSVFLFPPKPKDTLMISRTPEVTCVVVDVSHEDPEVQFNWYVDGVEVHNAKTKPR
EEQFNSTFRVVSVLTVVHQDWLNGKEYKCKVSNKGLPAPIEKTISKTKGQPREPQVY
TLPPSREEMTKNQVSLTCLVKGFYPSDIAVEWESNGQPENNYKTTPPMLDSDGSFFL
YSKLTVDKSRWQQGNVFSCSVMHEALHNHYTQKSLSLSPGK (SEQ ID NO: 55).
>Human Fc-IgG3
ASTKGPSVFPLAPCSRSTSGGTAALGCLVKDYFPEPVTVSWNSGALTSGVHTFPAVL
QSSGLYSLSSVVTVPSSSLGTQTYTCNVNHKPSNTKVDKRVELKTPLGDTTHTCPRC
PEPKSCDTPPPCPRCPEPKSCDTPPPCPRCPEPKSCDTPPPCPRCPAPELLGGPSVFLFPP
KPKDTLMISRTPEVTCVVVDVSHEDPEVQFKWYVDGVEVHNAKTKPREEQYNSTFR
VVSVLTVLHQDWLNGKEYKCKVSNKALPAPIEKTISKTKGQPREPQVYTLPPSREEM
TKNQVSLTCLVKGFYPSDIAVEWESSGQPENNYNTTPPMLDSDGSFFLYSKLTVDKS
RWQQGNIFSCSVMHEALHNRFTQKSLSLSPGK(SEQ ID NO: 56)
>Human Fc-IgG4
ASTKGPSVFPLAPCSRSTSESTAALGCLVKDYFPEPVTVSWNSGALTSGVHTFPAVLQS
SGLYSLSSVVTVPSSSLGTKTYTCNVDHKPSNTKVDKRVESKYGPPCPSCPAPEFLGG
PSVFLFPPKPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSDGSFFLYSR
LTVDKSRWQEGNVFSCSVMHEALHNHYTQKSLSLSLG(SEQ ID NO: 57).
>Human Fc-IgG4 5228P
ASTKGPSVFPLAPCSRSTSESTAALGCLVKDYFPEPVTVSWNSGALTSGVHTFPAVLQS
SGLYSLSSVVTVPSSSLGTKTYTCNVDHKPSNTKVDKRVESKYGPPCPPCPAPEFLGG
PSVFLFPPKPKDTLMISRTPEVTCVVVDVSQEDPEVQFNWYVDGVEVHNAKTKPREE
QFNSTYRVVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSDGSFFLYSR
LTVDKSRWQEGNVFSCSVMHEALHNHYTQKSLSLSLG (SEQ ID NO: 58).
>Human Fc-IgG4 PE
ASTKGPSVFPLAPCSRSTSESTAALGCLVKDYFPEPVTVSWNSGALTSGVHTFPAVLQ
SSGLYSLSSVVTVPSSSLGTKTYTCNVDHKPSNTKVDKRVESKYGPPCPPCPAPEFEG
GPSVFLFPPKPKDTLMISRTPEVTCVVVDVSQEDPEVQFNWYVDGVEVHNAKTKPR
EEQFNSTYRVVSVLTVLHQDWLNGKEYKCKVSNKGLPSSIEKTISKAKGQPREPQVY
TLPPSQEEMTKNQVSLTCLVKGFYPSDIAVEWESNGQPENNYKTTPPVLDSDGSFFL
YSRLTVDKSRWQEGNVFSCSVMHEALHNHYTQKSLSLSLGK (SEQ ID NO: 59)
>Human Fc-IgG4 PE'
ASTKGPSVFPLAPCSRSTSESTAALGCLVKDYFPEPVTVSWNSGALTSGVHTFPAVLQ
SS GLYSLSSVVTVPSSSLGTKTYTCNVDHKPSNTKVDKRVESKYGPPCPPCPAPEFEG
GPSVFLFPPKPKDTLMISRTPEVTCVVVDVSQEDPEVQFNWYVDGVEVHNAKTKPR
EEQFNSTYRVVSVLTVLHQDWLNGKEYKCKVSNKGLPSSIEKTISKAKGQPREPQVY
TLPPSQEEMTKNQVSLTCLVKGFYPSDIAVEWESNGQPENNYKTTPPVLDSDGSFFL
YSRLTVDKSRWQEGNVFSCSVMHEALHNHYTQKSLSLSLG (SEQ ID NO: 99)
>Human kappa LC
RTVAAPSVFIFPPSDEQLKSGTASVVCLLNNFYPREAKVQWKVDNALQSGNSQESVT
EQDSKDSTYSLSSTLTLSKADYEKHKVYACEVTHQGLSSPVTKSFNRGEC (SEQ ID
NO: 60).
>Rat Fc-IgG2c
ARTTAPSVYPLVPGCSGTSGSLVTLGCLVKGYFPEPVTVKWNSGALSSGVHTFPAVL
QSGLYTLSSSVTVPSSTWSSQTVTCSVAHPATKSNLIKRIEPRRPKPRPPTDICSCDDN
LGRPSVFIFPPKPKDILMITLTPKVTCVVVDVSEEEPDVQFSWFVDNVRVFTAQTQPH
EEQLNGTFRVVSTLHIQHQDWMSGKEFKCKVNNKDLPSPIEKTISKPRGKARTPQVY
TIPPPREQMSKNKVSLTCMVTSFYPASISVEWERNGELEQDYKNTLPVLDSDESYFLY
SKLSVDTDSWMRGDIYTCSVVHEALHNHHTQKNLSRSPGK (SEQ ID NO: 61).
>Rat kappa LC
RADAAPTVSIFPPSMEQLTSGGATVVCFVNNFYPRDISVKWKIDGSEQRDGVLDSVT
DQDSKDSTYSMSSTLSLTKVEYERHNLYTCEVVHKTSSSPVVKSFNRNEC (SEQ ID
NO: 62).
Rabbit IgG-Fc
>Rabbit IgG
GQPKAPSVFPLAPCCGDTPSSTVTLGCLVKGYLPEPVTVTWNSGTLTNGVRTFPSVR
QSSGLYSLSSVVSVTSSSQPVTCNVAHPATNTKVDKTVAPSTCSKPTCPPPELLGGPS
VFIFPPKPKDTLMISRTPEVTCVVVDVSQDDPEVQFTWYINNEQVRTARPPLREQQFN
STIRVVSTLPIAHQDWLRGKEFKCKVHNKALPAPIEKTISKARGQPLEPKVYTMGPPR
EELSSRSVSLTCMINGFYPSDISVEWEKNGKAEDNYKTTPAVLDSDGSYFLYSKLSVP
TSEWQRGDVFTCSVMHEALHNHYTQKSISRSPGK (SEQ ID NO: 63).
>Rabbit kappa LC
RDPVAPTVLIFPPAADQVATGTVTIVCVANKYFPDVTVTWEVDGTTQTTGIENSKTP
QNSADCTYNLSSTLTLTSTQYNSHKEYTCKVTQGTTSVVQSFNRGDC (SEQ ID
NO: 64).
>CD47
MWPLVAALLLGSACCGSAQLLFNKTKSVEFTFCNDTVVIPCFVTNMEAQNTTEVYV
KWKFKGRDIYTFDGALNKSTVPTDFSSAKIEVSQLLKGDASLKMDKSDAVSHTGNY
TCEVTELTREGETIIELKYRVVSWFSPNENILIVIFPIFAILLFWGQFGIKTLKYRSGGM
DEKTIALLVAGLVITVIVIVGAILFVPGEYSLKNATGLGLIVTSTGILILLHYYVFSTAIG
LTSFVIAILVIQVIAYILAVVGLSLCIAACIPMHGPLLISGLSILALAQLLGLVYMKFVE
(SEQ ID NO: 65).
Chimera and Human Light Chains
>Vx4murL01 Full length
DVLMTQTPLSLPVNLGDQASISCRSRQSIVHTNGNTYLGWFLQKPGQSPKLLIYKVS
NRFSGVPDRFSGSGSGTDFTLTISRVEAEDLGVYYCFQGSHVPYTFGGGTKLEIKRTV
AAPSVFIFPPSDEQLKSGTASVVCLLNNFYPREAKVQWKVDNALQSGNSQESVTEQD
SKDSTYSLSSTLTLSKADYEKHKVYACEVTHQGLSSPVTKSFNRGEC (SEQ ID
NO: 66).
>Vx4murL01 Full length
DVLMTQTPLSLPVNLGDQASISCRSRQSIVHTNGNTYLGWFLQKPGQSPKLLIYKVS
NRFSGVPDRFSGSGSGTDFTLTISRVEAEDLGVYYCFQGSHVPYTFGQGTKVEIKRTV
AAPSVFIFPPSDEQLKSGTASVVCLLNNFYPREAKVQWKVDNALQSGNSQESVTEQD
SKDSTYSLSSTLTLSKADYEKHKVYACEVTHQGLSSPVTKSFNRGEC (SEQ ID NO: 67).
>Vx4humL01 Full length LC
DIVMTQSPLSLPVTPGEPASISCRSRQSIVHTNGNTYLGWYLQKPGQSPRLLIYKVSN
RFSGVPDRFSGSGSGTDFTLKISRVEADDVGIYYCFQGSHVPYTFGQGTKLEIKRTVA
APSVFIFPPSDEQLKSGTASVVCLLNNFYPREAKVQWKVDNALQSGNSQESVTEQDS
KDSTYSLSSTLTLSKADYEKHKVYACEVTHQGLSSPVTKSFNRGEC (SEQ ID NO: 68).
>Vx8humL03 Full length LC
DIVMTQSPLSLPVTPGEPASISCRASQDISNYLNVVYLQKPGQSPRLLIYYTSRLYSGVP
DRFSGSGSGTDFTLKISRVEADDVGIYYCQQGNTLPWTFGQGTKLEIKRTVAAPSVFI
FPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC (SEQ ID NO: 69).
>Vx9humL02 Full length LC
DIVMTQSPDSLAVSLGERATINCRSSQNIVQSNGNTYLEWYQQKPGQPPKLLIYKVF
HRFSGVPDRFSGSGSGTDFTLTISSLQAEDVAVYYCFQGSHVPYTFGQGTKLEIKRTV
AAPSVFIFPPSDEQLKSGTASVVCLLNNFYPREAKVQWKVDNALQSGNSQESVTEQD
SKDSTYSLSSTLTLSKADYEKHKVYACEVTHQGLSSPVTKSFNRGEC (SEQ ID
NO: 70).
>Vx8humL02 Full length LC
DIQMTQSPSSLSASVGDRVTITCRASQSISNYLNWYQQKPGKAPKLLIYYTSRLYSGV
PSRFSGSGSGTDFTLTISSLQPEDFATYYCQQGNTLPWTFGQGTKVEIKRTVAAPSVFI
FPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC (SEQ ID NO: 71).
>Vx4humL02 Full length LC
DVVMTQSPLSLPVTLGQPASISCRSRQSIVHTNGNTYLGWFQQRPGQSPRRLIYKVSN
RFSGVPDRFSGSGSGTDFTLKISRVEAEDVGVYYCFQGSHVPYTFGQGTKLEIKRTVA
APSVFIFPPSDEQLKSGTASVVCLLNNFYPREAKVQWKVDNALQSGNSQESVTEQDS
KDSTYSLSSTLTLSKADYEKHKVYACEVTHQGLSSPVTKSFNRGEC (SEQ ID NO: 72).
>Vx9humL07 Full length LC
DVVMTQSPLSLPVTLGQPASISCRSSQNIVQSNGNTYLEWFQQRPGQSPRRLIYKVFH
RFSGVPDRFSGSGSGTDFTLKISRVEAEDVGVYYCFQGSHVPYTFGQGTKLEIKRTVA
APSVFIFPPSDEQLKSGTASVVCLLNNFYPREAKVQWKVDNALQSGNSQESVTEQDS
KDSTYSLSSTLTLSKADYEKHKVYACEVTHQGLSSPVTKSFNRGEC (SEQ ID NO: 73).
>Vx8humL01 Full length LC
DIQMTQSPSSLSASVGDRVTITCRASQDISNYLNWYQQKPGKAPKLLIYYTSRLYSGV
PSRFSGSGSGTDFTFTISSLQPEDIATYYCQQGNTLPWTFGQGTKVEIKRTVAAPSVFI
FPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC (SEQ ID NO: 74).
>Vx8murL03 Full length LC
DIQMTQTTSSLSASLGDRVTISCRASQDISNYLNWYQQKPDGTVKLLIYYTSRLYSGV
PSRFSGSGSGTDYSLTISNLEQEDIATYFCQQGNTLPWTFGGGTKLEIKRTVAAPSVFI
FPPSDEQLKSGTASVVCLLNNFYPREAKVQWKVDNALQSGNSQESVTEQDSKDSTY
SLSSTLTLSKADYEKHKVYACEVTHQGLSSPVTKSFNRGEC (SEQ ID NO: 100).
>Vx9mur LO4 Full length LC
DVFMTQTPLSLPVSLGDQASISCRSSQNIVQSNGNTYLEWYLQKPGQSPKLLIYKVFH
RFSGVPDRFSGSGSGTDFTLKISRVEAEDLGVYYCFQGSHVPWTFGGGTKVEIKRTV
AAPSVFIFPPSDEQLKSGTASVVCLLNNFYPREAKVQWKVDNALQSGNSQESVTEQD
SKDSTYSLSSTLTLSKADYEKHKVYACEVTHQGLSSPVTKSFNRGEC (SEQ ID
NO: 75).
Chimera and Human Heavy Chains
>Vx4murH01 Full length HC
EVQLQQSGPELVKPGASVKMSCKASGYTFTNYVIHWVKRRPGQGLEWIGYIYPYND
GILYNEKFKGKATVTSDKSSSTAYMDLSSLTSEDSAVYYCTRGGYYVPDYWGQGTTL
TVSS
ASTKGPSVFPLAPCSRSTSESTAALGCLVKDYFPEPVTVSWNSGALTSGVHTFPAVLQ
SSGLYSLSSVVTVPSSSLGTKTYTCNVDHKPSNTKVDKRVESKYGPPCPPCPAPEFEG
GPSVFLFPPKPKDTLMISRTPEVTCVVVDVSQEDPEVQFNWYVDGVEVHNAKTKPR
EEQFNSTYRVVSVLTVLHQDWLNGKEYKCKVSNKGLPSSIEKTISKAKGQPREPQVY
TLPPSQEEMTKNQVSLTCLVKGFYP SDIAVEWESNGQPENNYKTTPPVLDSDGSFFL
YSRLTVDKSRWQEGNVFSCSVMHEALHNHYTQKSLSLSLGK (SEQ ID NO: 76).
>Vx4humH01 Full length HC
QVQLVQSGAEVKKPGASVQVSCKASGYTFTNYVIHWLRQAPGQGLEWMGYIYPYN
DGILYNEKFKGRVTMTSDTSISTAYMELSSLRSDDTAVYYCARGGYYVPDYVVGQAT
LVTVSSASTKGPSVFPLAPCSRSTSESTAALGCLVKDYFPEPVTVSWNSGALTSGVHT
FPAVLQSSGLYSLSSVVTVPSSSLGTKTYTCNVDHKPSNTKVDKRVESKYGPPCPPCP
APEFEGGPSVFLFPPKPKDTLMISRTPEVTCVVVDVSQEDPEVQFNWYVDGVEVHNA
KTKPREEQFNSTYRVVSVLTVLHQDWLNGKEYKCKVSNKGLPSSIEKTISKAKGQPR
EPQVYTLPPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGK (SEQ ID
NO: 77).
>Vx8humH11 Full length HC
QVQLVQSGAEVKKPGASVQVSCKASGYSFTNYYIHWLRQAPGQGLEWMGYIDPLN
GDTTYNQKFKGRVTMTSDTSISTAYMELSSLRSDDTAVYYCARGGKRAMDYWGQAT
LVTVSSASTKGPSVFPLAPCSRSTSESTAALGCLVKDYFPEPVTVSWNSGALTSGVHT
FPAVLQSSGLYSLSSVVTVPSSSLGTKTYTCNVDHKPSNTKVDKRVESKYGPPCPPCP
APEFEGGPSVFLFPPKPKDTLMISRTPEVTCVVVDVSQEDPEVQFNWYVDGVEVHNA
KTKPREEQFNSTYRVVSVLTVLHQDWLNGKEYKCKVSNKGLPSSIEKTISKAKGQPR
EPQVYTLPPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGK (SEQ ID
NO: 78).
>Vx9humH12 Full length HC
QVQLVQSGAEVKKPGASVKVSCKASGYTFTNYWIHWVRQAPGQGLEWMGYTDPR
TDYTEYNQKFKDRVTMTRDTSTSTVYMELSSLRSEDTAVYYCARGGRVGLGYWGQ
GTLVTVSSASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVERKCCVECP
PCPAPPVAGPSVFLFPPKPKDTLMISRTPEVTCVVVDVSHEDPEVQFNWYVDGVEVH
NAKTKPREEQFNSTFRVVSVLTVVHQDWLNGKEYKCKVSNKGLPAPIEKTISKTKG
QPREPQVYTLPPSREEMTKNQVSLTCLVKGFYPSDIAVEWESNGQPENNYKTTPPML
DSDGSFFLYSKLTVDKSRWQQGNVFSCSVMHEALHNHYTQKSLSLSPGK (SEQ ID
NO: 79).
>Vx9humH14 Full length HC
EVQLVQSGAEVKKPGESLKISCKGSGYTFTNYWIHWVRQMPGKGLEWMGYTDPRT
DYTEYNQKFKDQVTISADKSISTAYLQWSSLKASDTAMYYCARGGRVGLGYWGQG
TLVTVSSASTKGPSVFPLAPCSRSTSESTAALGCLVKDYFPEPVTVSWNSGALTSGVH
TFPAVLQSSGLYSLSSVVTVPSSNFGTQTYTCNVDHKPSNTKVDKTVERKCCVECPP
CPAPPVAGPSVFLFPPKPKDTLMISRTPEVTCVVVDVSHEDPEVQFNVVYVDGVEVHN
AKTKPREEQFNSTFRVVSVLTVVHQDWLNGKEYKCKVSNKGLPAPIEKTISKTKGQP
REPQVYTLPPSREEMTKNQVSLTCLVKGFYPSDIAVEWESNGQPENNYKTTPPMLDS
DGSFFLYSKLTVDKSRWQQGNVFSCSVMHEALHNHYTQKSLSLSPGK (SEQ ID
NO: 80).
>Vx9humH15 Full length HC
QVQLVQSGAEVKKPGSSVKVSCKASGYTFTNYVVIHWVRQAPGQGLEWMGYTDPRT
DYTEYAQKFQGRVTITADESTSTAYMELSSLRSEDTAVYYCARGGRVGLGYWGQGTL
VTVSSASTKGPSVFPLAPCSRSTSESTAALGCLVKDYFPEPVTVSWNSGALTSGVHTF
PAVLQSSGLYSLSSVVTVPSSNFGTQTYTCNVDHKPSNTKVDKTVERKCCVECPPCP
APPVAGPSVFLFPPKPKDTLMISRTPEVTCVVVDVSHEDPEVQFNWYVDGVEVHNA
KTKPREEQFNSTFRVVSVLTVVHQDWLNGKEYKCKVSNKGLPAPIEKTISKTKGQPR
EPQVYTLPPSREEMTKNQVSLTCLVKGFYPSDIAVEWESNGQPENNYKTTPPMLDSD
GSFFLYSKLTVDKSRWQQGNVFSCSVMHEALHNHYTQKSLSLSPGK (SEQ ID
NO: 81).
>Vx4humH02 Full length HC
QVQLVQSGAEVKKPGASVQVSCKASGYTFTNYVIHWLRQAPGQGLEWMGYIYPYN
DGILYNEKFKGRVTMTSDTSISTAYMELSSLRSDDTAVYYCARGGYYVYDYWGQATL
VTVSSASTKGPSVFPLAPCSRSTSESTAALGCLVKDYFPEPVTVSWNSGALTSGVHTF
PAVLQSSGLYSLSSVVTVPSSSLGTKTYTCNVDHKPSNTKVDKRVESKYGPPCPPCPA
PEFEGGPSVFLFPPKPKDTLMISRTPEVTCVVVDVSQEDPEVQFNWYVDGVEVHNAK
TKPREEQFNSTYRVVSVLTVLHQDWLNGKEYKCKVSNKGLPS SIEKTISKAKGQPRE
PQVYTLPPSQEEMTKNQVSLTCLVKGFYPSDIAVEWESNGQPENNYKTTPPVLDSDG
SFFLYSRLTVDKSRWQEGNVFSCSVMHEALHNHYTQKSLSLSLGK (SEQ ID NO: 82).
>Vx9humH13 Full length HC
QVQLVQSGAEVKKPGSSVKVSCKASGYTFTNYWIHWVRQAPGQGLEWMGYTDPRT
DYTEYNQKFKDRVTITADESTSTAYMELSSLRSEDTAVYYCARGGRVGLGYWGQGTL
VTVSSASTKGPSVFPLAPCSRSTSESTAALGCLVKDYFPEPVTVSWNSGALTSGVHTF
PAVLQSSGLYSLSSVVTVPSSNFGTQTYTCNVDHKPSNTKVDKTVERKCCVECPPCP
APPVAGPSVFLFPPKPKDTLMISRTPEVTCVVVDVSHEDPEVQFNWYVDGVEVHNA
KTKPREEQFNSTFRVVSVLTVVHQDWLNGKEYKCKVSNKGLPAPIEKTISKTKGQPR
EPQVYTLPPSREEMTKNQVSLTCLVKGFYPSDIAVEWESNGQPENNYKTTPPMLDSD
GSFFLYSKLTVDKSRWQQGNVFSCSVMHEALHNHYTQKSLSLSPGK (SEQ ID
NO: 83).
>Vx8humH10 Full length HC
EVQLVQSGAEVKKPGESLKISCKGSGYSFTNYYIHWVRQMPGKGLEWMGYIDPLNG
DTTYSPSFQGQVTISADKSISTAYLQWSSLKASDTAMYYCARGGKRAMDYWGRGTL
VTVSSASTKGPSVFPLAPCSRSTSESTAALGCLVKDYFPEPVTVSWNSGALTSGVHTF
PAVLQSSGLYSLSSVVTVPSSSLGTKTYTCNVDHKPSNTKVDKRVESKYGPPCPPCPA
PEFEGGPSVFLFPPKPKDTLMISRTPEVTCVVVDVSQEDPEVQFNWYVDGVEVHNAK
TKPREEQFNSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTTPPVLDSDG
SFFLYSRLTVDKSRWQEGNVFSCSVMHEALHNHYTQKSLSLSLGK (SEQ ID NO: 84).
>Vx4humH04 Full length HC
EVQLVQSGAEVKKPGESLKISCKGSGYTFTNYVIHWVRQMPGKGLEWMGYIYPYND
GILYNEKFKGQVTISADKSISTAYLQWSSLKASDTAMYYCARGGYYVPDYWGQGTT
VTVSSASTKGPSVFPLAPCSRSTSESTAALGCLVKDYFPEPVTVSWNSGALTSGVHTF
PAVLQSSGLYSLSSVVTVPSSSLGTKTYTCNVDHKPSNTKVDKRVESKYGPPCPPCPA
PEFEGGPSVFLFPPKPKDTLMISRTPEVTCVVVDVSQEDPEVQFNWYVDGVEVHNAK
TKPREEQFNSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTTPPVLDSDG
SFFLYSRLTVDKSRWQEGNVFSCSVMHEALHNHYTQKSLSLSLGK (SEQ ID NO: 85).
>Vx4humH05 Full length HC
QVQLVQSGAEVKKPGASVKVSCKASGYTFTNYVIHWVRQAPGQGLEWMGYIYPYN
DGILYNEKFKGRVTMTTDTSTSTAYMELRSLRSDDTAVYYCARGGYYVPDYWGQGT
TVTVSSASTKGPSVFPLAPCSRSTSESTAALGCLVKDYFPEPVTVSWNSGALTSGVHT
FPAVLQSSGLYSLSSVVTVPSSSLGTKTYTCNVDHKPSNTKVDKRVESKYGPPCPPCP
APEFEGGPSVFLFPPKPKDTLMISRTPEVTCVVVDVSQEDPEVQFNWYVDGVEVHNA
KTKPREEQFNSTYRVVSVLTVLHQDWLNGKEYKCKVSNKGLPSSIEKTISKAKGQPR
EPQVYTLPPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGK (SEQ ID
NO: 86).
>Vx9humH16 Full length HC
EVQLVQSGAEVKKPGESLKISCKGSGYTFTNYWIHWVRQMPGKGLEWMGYTDPRT
DYTEYSPSFQGQVTISADKSISTAYLQWSSLKASDTAMYYCARGGRVGLGYWGQGT
LVTVSSASTKGPSVFPLAPCSRSTSESTAALGCLVKDYFPEPVTVSWNSGALTSGVHT
FPAVLQSSGLYSLSSVVTVPSSNFGTQTYTCNVDHKPSNTKVDKTVERKCCVECPPC
PAPPVAGPSVFLFPPKPKDTLMISRTPEVTCVVVDVSHEDPEVQFNWYVDGVEVHNA
KTKPREEQFNSTFRVVSVLTVVHQDWLNGKEYKCKVSNKGLPAPIEKTISKTKGQPR
EPQVYTLPPSREEMTKNQVSLTCLVKGFYPSDIAVEWESNGQPENNYKTTPPMLDSD
GSFFLYSKLTVDKSRWQQGNVFSCSVMHEALHNHYTQKSLSLSPGK (SEQ ID
NO: 87).
>Vx8humH06 Full length HC
QVQLVQSGAEVKKPGASVKVSCKASGYSFTNYYIHWVRQAPGQGLEWMGYIDPLN
GDTTYNQKFKGRVTMTRDTSTSTVYMELSSLRSEDTAVYYCARGGKRAMDYWGQG
TLVTVSSASTKGPSVFPLAPCSRSTSESTAALGCLVKDYFPEPVTVSWNSGALTSGVH
TFPAVLQSSGLYSLSSVVTVPSSSLGTKTYTCNVDHKPSNTKVDKRVESKYGPPCPPC
PAPEFEGGPSVFLFPPKPKDTLMISRTPEVTCVVVDVSQEDPEVQFNVVYVDGVEVHN
AKTKPREEQFNSTYRVVSVLTVLHQDWLNGKEYKCKVSNKGLPSSIEKTISKAKGQP
REPQVYTLPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGK (SEQ ID
NO: 88).
>Vx8humH07 Full length HC
QVQLVQSGAEVKKPGSSVKVSCKASGYSFTNYYIHWVRQAPGQGLEWMGYIDPLN
GDTTYNQKFKGRVTITADESTSTAYMELSSLRSEDTAVYYCARGGKRAMDYWGQGT
LVTVSSASTKGPSVFPLAPCSRSTSESTAALGCLVKDYFPEPVTVSWNSGALTSGVHT
FPAVLQSSGLYSLSSVVTVPSSSLGTKTYTCNVDHKPSNTKVDKRVESKYGPPCPPCP
APEFEGGPSVFLFPPKPKDTLMISRTPEVTCVVVDVSQEDPEVQFNWYVDGVEVHNA
KTKPREEQFNSTYRVVSVLTVLHQDWLNGKEYKCKVSNKGLPSSIEKTISKAKGQPR
EPQVYTLPPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGK (SEQ ID
NO: 89).
>Vx8humH08 Full length HC
EVQLVQSGAEVKKPGESLKISCKGSGYSFTNYYIHWVRQMPGKGLEWMGYIDPLNG
DTTYNQKFKGQVTISADKSISTAYLQWSSLKASDTAMYYCARGGKRAMDYWGQGT
LVTVSSASTKGPSVFPLAPCSRSTSESTAALGCLVKDYFPEPVTVSWNSGALTSGVHT
FPAVLQSSGLYSLSSVVTVPSSSLGTKTYTCNVDHKPSNTKVDKRVESKYGPPCPPCP
APEFEGGPSVFLFPPKPKDTLMISRTPEVTCVVVDVSQEDPEVQFNWYVDGVEVHNA
KTKPREEQFNSTYRVVSVLTVLHQDWLNGKEYKCKVSNKGLPSSIEKTISKAKGQPR
EPQVYTLPPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGK (SEQ ID
NO: 90).
>Vx8humH09 Full length HC
QVQLVQSGAEVKKPGSSVKVSCKASGYSFTNYYIHWVRQAPGQGLEWMGYIDPLN
GDTTYAQKFQGRVTITADESTSTAYMELSSLRSEDTAVYYCARGGKRAMDYWGQGT
LVTVSSASTKGPSVFPLAPCSRSTSESTAALGCLVKDYFPEPVTVSWNSGALTSGVHT
FPAVLQSSGLYSLSSVVTVPSSSLGTKTYTCNVDHKPSNTKVDKRVESKYGPPCPPCP
APEFEGGPSVFLFPPKPKDTLMISRTPEVTCVVVDVSQEDPEVQFNWYVDGVEVHNA
KTKPREEQFNSTYRVVSVLTVLHQDWLNGKEYKCKVSNKGLPSSIEKTISKAKGQPR
EPQVYTLPPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGK (SEQ ID
NO: 91).
>Vx8humH06 Full length HC
QVQLVQSGAEVKKPGASVKVSCKASGYSFTNYYIHWVRQAPGQGLEWMGYIDPLN
GDTTYNQKFKGRVTMTRDTSTSTVYMELSSLRSEDTAVYYCARGGKRAMDYWGQG
TLVTVSSASTKGPSVFPLAPCSRSTSESTAALGCLVKDYFPEPVTVSWNSGALTSGVH
TFPAVLQSSGLYSLSSVVTVPSSSLGTKTYTCNVDHKPSNTKVDKRVESKYGPPCPPC
PAPEFEGGPSVFLFPPKPKDTLMISRTPEVTCVVVDVSQEDPEVQFNVVYVDGVEVHN
AKTKPREEQFNSTYRVVSVLTVLHQDWLNGKEYKCKVSNKGLPSSIEKTISKAKGQP
REPQVYTLPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGK
(SEQ ID NO: 92).
>Vx8mur-H03 Full length HC
EVQLQQSGPELMKPGASVKISCKASGYSFTNYYIHWVNQSHGKSLEWIGYIDPLNGD
TTYNQKFKGKATLTVDKSSSTAYMRLSSLTSADSAVYYCARGGKRAMDYWGQGTSV
TVSS
ASTKGPSVFPLAPCSRSTSESTAALGCLVKDYFPEPVTVSWNSGALTSGVHTFPAVLQ
SSGLYSLSSVVTVPSSSLGTKTYTCNVDHKPSNTKVDKRVESKYGPPCPPCPAPEFEG
GPSVFLFPPKPKDTLMISRTPEVTCVVVDVSQEDPEVQFNWYVDGVEVHNAKTKPR
EEQFNSTYRVVSVLTVLHQDWLNGKEYKCKVSNKGLPSSIEKTISKAKGQPREPQVY
TLPPSQEEMTKNQVSLTCLVKGFYPSDIAVEWESNGQPENNYKTTPPVLDSDGSFFL
YSRLTVDKSRWQEGNVFSCSVMHEALHNHYTQKSLSLSLGK (SEQ ID NO: 93).
>Vx9mur-H04 Full length HC
QVQLQQFGAELAKPGASVQMSCKASGYTFTNYWIHWVKQRPGQGLEWIGYTDPRT
DYTEYNQKFKDKATLAADRSSSTAYMRLSSLTSEDSAVYYCAGGGRVGLGYWGHGS
SVTVSSASTKGPSVFPLAPCSRSTSESTAALGCLVKDYFPEPVTVSWNSGALTSGVHT
FPAVLQSSGLYSLSSVVTVPSSNFGTQTYTCNVDHKPSNTKVDKTVERKCCVECPPC
PAPPVAGPSVFLFPPKPKDTLMISRTPEVTCVVVDVSHEDPEVQFNWYVDGVEVHNA
KTKPREEQFNSTFRVVSVLTVVHQDWLNGKEYKCKVSNKGLPAPIEKTISKTKGQPR
EPQVYTLPPSREEMTKNQVSLTCLVKGFYPSDIAVEWESNGQPENNYKTTPPMLDSD
GSFFLYSKLTVDKSRWQQGNVFSCSVMHEALHNHYTQKSLSLSPGK
(SEQ ID NO: 94).
>Vx8humH06 Full length HC
QVQLVQSGAEVKKPGASVKVSCKASGYSFTNYYIHWVRQAPGQGLEWMGYIDPLN
GDTTYNQKFKGRVTMTRDTSTSTVYMELSSLRSEDTAVYYCARGGKRAMDYWGQG
TLVTVSSASTKGPSVFPLAPCSRSTSESTAALGCLVKDYFPEPVTVSWNSGALTSGVH
TFPAVLQSSGLYSLSSVVTVPSSNFGTQTYTCNVDHKPSNTKVDKTVERKCCVECPP
CPAPPVAGPSVFLFPPKPKDTLMISRTPEVTCVVVDVSHEDPEVQFNVVYVDGVEVHN
AKTKPREEQFNSTFRVVSVLTVVHQDWLNGKEYKCKVSNKGLPAPIEKTISKTKGQP
REPQVYTLPPSREEMTKNQVSLTCLVKGFYPSDIAVEWESNGQPENNYKTTPPMLDS
DGSFFLYSKLTVDKSRWQQGNVFSCSVMHEALHNHYTQKSLSLSPGK (SEQ ID
NO: 95).
>Vx8humH07 Full length HC
QVQLVQSGAEVKKPGSSVKVSCKASGYSFTNYYIHWVRQAPGQGLEWMGYIDPLN
GDTTYNQKFKGRVTITADESTSTAYMELSSLRSEDTAVYYCARGGKRAMDYWGQGT
LVTVSSASTKGPSVFPLAPCSRSTSESTAALGCLVKDYFPEPVTVSWNS GALTSGVHT
FPAVLQSSGLYSLSSVVTVPSSNFGTQTYTCNVDHKPSNTKVDKTVERKCCVECPPC
PAPPVAGPSVFLFPPKPKDTLMISRTPEVTCVVVDVSHEDPEVQFNWYVDGVEVHNA
KTKPREEQFNSTFRVVSVLTVVHQDWLNGKEYKCKVSNKGLPAPIEKTISKTKGQPR
EPQVYTLPPSREEMTKNQVSLTCLVKGFYPSDIAVEWESNGQPENNYKTTPPMLDSD
GSFFLYSKLTVDKSRWQQGNVFSCSVMHEALHNHYTQKSLSLSPGK (SEQ ID
NO: 96).
>Vx8humH08 Full length HC
EVQLVQSGAEVKKPGESLKISCKGSGYSFTNYYIHWVRQMPGKGLEWMGYIDPLNG
DTTYNQKFKGQVTISADKSISTAYLQWSSLKASDTAMYYCARGGKRAMDYWGQGT
LVTVSSASTKGPSVFPLAPCSRSTSESTAALGCLVKDYFPEPVTVSWNSGALTSGVHT
FPAVLQSSGLYSLSSVVTVPSSNFGTQTYTCNVDHKPSNTKVDKTVERKCCVECPPC
PAPPVAGPSVFLFPPKPKDTLMISRTPEVTCVVVDVSHEDPEVQFNWYVDGVEVHNA
KTKPREEQFNSTFRVVSVLTVVHQDWLNGKEYKCKVSNKGLPAPIEKTISKTKGQPR
EPQVYTLPPSREEMTKNQVSLTCLVKGFYPSDIAVEWESNGQPENNYKTTPPMLDSD
GSFFLYSKLTVDKSRWQQGNVFSCSVMHEALHNHYTQKSLSLSPGK (SEQ ID
NO: 97).
>Vx8humH09 Full length HC
QVQLVQSGAEVKKPGSSVKVSCKASGYSFTNYYIHWVRQAPGQGLEWMGYIDPLN
GDTTYAQKFQGRVTITADESTSTAYMELSSLRSEDTAVYYCARGGKRAMDYWGQGT
LVTVSSASTKGPSVFPLAPCSRSTSESTAALGCLVKDYFPEPVTVSWNSGALTSGVHT
FPAVLQSSGLYSLSSVVTVPSSNFGTQTYTCNVDHKPSNTKVDKTVERKCCVECPPC
PAPPVAGPSVFLFPPKPKDTLMISRTPEVTCVVVDVSHEDPEVQFNWYVDGVEVHNA
KTKPREEQFNSTFRVVSVLTVVHQDWLNGKEYKCKVSNKGLPAPIEKTISKTKGQPR
EPQVYTLPPSREEMTKNQVSLTCLVKGFYPSDIAVEWESNGQPENNYKTTPPMLDSD
GSFFLYSKLTVDKSRWQQGNVFSCSVMHEALHNHYTQKSLSLSPGK (SEQ ID
NO: 98).
>Vx4mur-ratL01 Full length LC
DVLMTQTPLSLPVNLGDQASISCRSRQSIVHTNGNTYLGWFLQKPGQSPKLLIYKVS
NRFSGVPDRFSGSGSGTDFTLTISRVEAEDLGVYYCFQGSHVPYTFGGGTKLEIK
RADAAPTVSIFPPSMEQLTSGGATVVCFVNNFYPRDISVKWKIDGSEQRDGVLDSVT
DQDSKDSTYSMSSTLSLTKVEYERHNLYTCEVVHKTSSSPVVKSFNRNEC (SEQ ID
NO: 101).
>Vx4mur-ratH01 Full length HC
EVQLQQSGPELVKPGASVKMSCKASGYTFTNYVIHWVKRRPGQGLEWIGYIYPYND
GILYNEKFKGKATVTSDKSSSTAYMDLSSLTSEDSAVYYCTRGGYYVPDYWGQ GTTL
TVSS
ARTTAPSVYPLVPGCSGTSGSLVTLGCLVKGYFPEPVTVKWNSGALSSGVHTFPAVL
QSGLYTLSSSVTVPSSTWSSQTVTCSVAHPATKSNLIKRIEPRRPKPRPPTDICSCDDN
LGRPSVFIFPPKPKDILMITLTPKVTCVVVDVSEEEPDVQFSWFVDNVRVFTAQTQPH
EEQLNGTFRVVSTLHIQHQDWMSGKEFKCKVNNKDLPSPIEKTISKPRGKARTPQVY
TIPPPREQMSKNKVSLTCMVTSFYPASISVEWERNGELEQDYKNTLPVLDSDESYFLY
SKLSVDTDSWMRGDIYTCSVVHEALHNHHTQKNLSRSPGK (SEQ ID NO: 102).
>Vx4mur-rabL01 Full length LC
DVLMTQTPLSLPVNLGDQASISCRSRQ SIVHTNGNTYLGWFLQKPGQSPKLLIYKVS
NRFSGVPDRFSGSGSGTDFTLTISRVEAEDLGVYYCFQGSHVPYTFGGGTKLEIK
RDPVAPTVLIFPPAADQVATGTVTIVCVANKYFPDVTVTWEVDGTTQTTGIENSKTP
QNSADCTYNLSSTLTLTSTQYNSHKEYTCKVTQGTTSVVQSFNRGDC (SEQ ID
NO: 103).
>Vx4mur-rabH01 Full length HC
EVQLQQSGPELVKPGASVKMSCKASGYTFTNYVIHWVKRRPGQGLEWIGYIYPYND
GILYNEKFKGKATVTSDKSSSTAYMDLSSLTSEDSAVYYCTRGGYYVPDYWGQGTTL
TVSS
GQPKAPSVFPLAPCCGDTPSSTVTLGCLVKGYLPEPVTVTWNSGTLTNGVRTFPSVR
QSSGLYSLSSVVSVTSSSQPVTCNVAHPATNTKVDKTVAPSTCSKPTCPPPELLGGPS
VFIFPPKPKDTLMISRTPEVTCVVVDVSQDDPEVQFTWYINNEQVRTARPPLREQQFN
STIRVVSTLPIAHQDWLRGKEFKCKVHNKALPAPIEKTISKARGQPLEPKVYTMGPPR
EELSSRSVSLTCMINGFYPSDISVEWEKNGKAEDNYKTTPAVLDSDGSYFLYSKLSVP
TSEWQRGDVFTCSVMHEALHNHYTQKSISRSPGK (SEQ ID NO: 104).

### Example 2

### Production of CD47 Antibodies

Chimeric antibodies disclosed herein comprise a mouse heavy chain variable domain and a light chain variable domain combined with a human kappa or human Fc IgG1, IgG1-N297Q, IgG2, IgG4, IgG4 S228P, and IgG4 PE constant domains, respectively. These were designed to incorporate a secretion signal and cloned into a mammalian expression system, and transfected into CHO cells to generate chimeric (murine-human) antibodies. The chimeric variants were expressed as full-length IgG molecules, secreted into the medium, and purified using protein A.

As such, the humanized antibodies disclosed herein comprise frameworks derived from the human genome. The collection covers the diversity found in the human germ line sequences, yielding functionally expressed antibodies in vivo. The complementarity determining regions (CDRs) in the light and heavy chain variable regions of the murine and chimeric (murine-human) are described herein and were determined by following commonly accepted rules disclosed in “Protein Sequence and Structure Analysis of Antibody Variable Domains”, In: Antibody Engineering Lab Manual, eds. S. Duebel and R. Kontermann, Springer-Verlag, Heidelberg (2001)). The human light chain variable domains were then designed. The humanized variable domains were then combined with a secretion signal and human kappa and human Fc IgG1, IgG1-N297Q, IgG2, IgG3, IgG4 S228P and IgG4 PE constant domains, cloned into a mammalian expression system, and transfected into CHO cells to generate humanized mAbs. The humanized variants were expressed as full-length IgG molecules, secreted into the medium and purified using protein A.

A non-glycosylated version (IgG1-N297Q) was created by site directed mutagenesis of heavy chain position 297 to change the asparagine to glutamine (Human Fc IgG1-N297Q, SEQ ID NO:54). An IgG4 variant was created by site-directed mutagenesis at position 228 to change the serine to proline thereby preventing in vivo Fab arm exchange. An IgG4 double mutant was created by site-directed mutagenesis at positions 228 (serine to proline) and 235 (leucine to glutamate) to prevent Fab arm exchange and to further reduce Fc effector function. IgG2, IgG3, IgG4 S228P, and IgG4 PE isotypes were constructed by cloning the heavy chain variable domain in frame with the human IgG2, IgG3, IgG4 S228P, and IgG4PE constant domains (Human Fc-IgG2, SEQ ID NO:55 Human Fc-IgG3, SEQ ID NO:56; Human Fc-IgG4 S228P, SEQ ID NO:58; and Human Fc-IgG4 PE, SEQ ID NO:59).

### Example 3

### NanoString Analysis of Tumors Treated with Anti-CD47 Antibodies

The treatment of in vivo xenograft tumor models with anti-CD47 therapies yields varying results in efficacy. In some tumor models, anti-CD47 antibodies lead to tumor growth inhibition (termed “responsive” or “responder” tumors) whereas in other tumor models, no growth inhibition is observed (termed “non-responsive” or “non-responder” tumors). Two tumor xenograft models responsive to anti-CD47 therapy are multiple myeloma RPMI-8226 and B cell lymphoma Raji and two non-responsive xenograft tumor models are non-small cell lung cancer H1975 and head and neck FaDu. NanoString technology was used to profile each of these tumor models to identify biomarkers which can predict tumor responsiveness to anti-CD47 therapy and aid in selection of positive patient outcomes to treatment and secondly, to identify gene targets which would convert non-responsive tumors to responsive tumors by assessing human tumor and mouse infiltrating immune cell gene expression profiles and to select patients who may respond to treatment.

Cells were purchased at ATCC and maintained in RPMI-1640 supplemented with 10% Fetal Bovine Serum and 1% Penicillin/Streptomycin except for FaDu which were maintained in EMEM with the same supplements within a 5% CO2 atmosphere.

Female NSG (NOD-Cg-Prkdc^{scid}I12rg^{tmlWjl}/SzJ) at 5-6 weeks of age were used. Mice were acclimated prior to handling and housed in microisolator cages under specific pathogen-free conditions. NSG mice were inoculated subcutaneously in the right flank with 0.1 ml of a 30% RPMI/70% Matrigel™(BD Biosciences; Bedford, Mass.) mixture containing a suspension of RPMI-8226 tumor cells, Raji, FaDu, or H1975 tumor cells. Tumor bearing animals were monitored and tumors were measured periodically until they reached designated start size of 200-250 mm³. Test articles were either control IgG (Control IgG) or anti-CD47 antibody administered by intravenous (IV) injection in the Raji model or intraperitoneal injection (IP) in the RPMI-8226, FaDu, or H1975 models. Control IgG and anti-CD47 mAb were administered at a dose of 25 mg/kg on day 0. Tumors were collected at 96 hours following dosing for Raji and H1975 and seven days following dosing for RPMI-8226 and FaDu xenograft models to achieve equivalent tumor loads.

Tumors were snap frozen and RNA was isolated, quantitated, and analyzed on the NanoString Mouse Myeloid panel and the NanoString Human PanCancer IO360 panel using the nCounter Max System. The Mouse Myeloid panel was used to profile the infiltrating murine immune cells and the Human PanCancer IO360 panel was used to profile the human tumor cells. Both panels include 20 housekeeping genes. All samples passed quality control which requires a Field of Views above 75%, binding density between 0.05 and 2.25 spots per square micron, positive control linearity with a correlation coefficient above 0.95, and an fM detection threshold of 0.5 fM positive control probes producing raw counts higher than the mean of the negative control probes. In both panels, six internal positive controls and eight internal negative controls were included. Samples were normalized to housekeeping genes and reported as NanoString units.

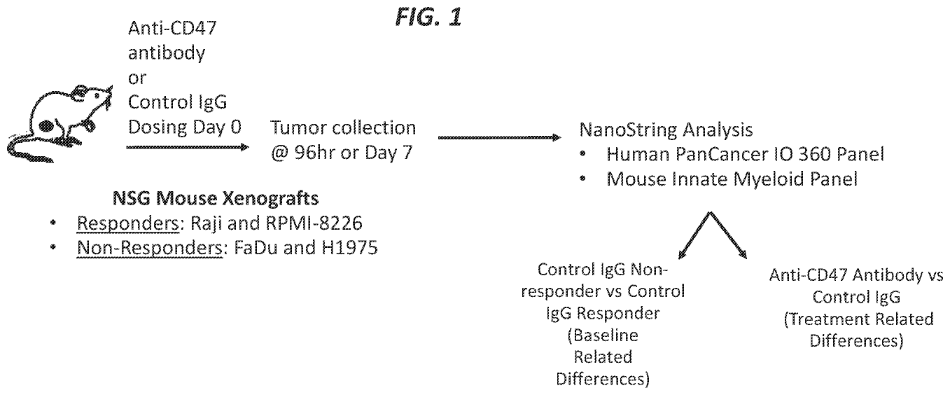

FIG. 1 shows the xenograft model workflow for all four tumor models assessed including implantation, treatment, tumor collection, RNA isolation, NanoString and analysis. Fold change and p values were calculated from the results of the Mouse Myeloid Panel and IO360 Human Pan Cancer Panels for an anti-CD47 mAb treated vs Control IgG treated groups for each of the tumor models tested. A summary of the findings are shown in Table 1 which compare differentially expressed genes in human tumor cells and mouse immune infiltrates following anti-CD47 or control IgG treatment.

TABLE 1
Analysis of NanoString Data Showing Genes Differentially Expressed
in Human Xenograft Tumor Cells and Murine Tumor Microenvironment
Following Treatment with anti-CD47 mAh vs control IgG.

| | Tumor Cells (Human) | | | |
|---|---|---|---|---|
| | Raji anti-CD47 | RPMI-8226 anti-CD47 | FaDu anti-CD47 | H1975 anti-CD47 |
| No. of Genes | mAb/control IgG | mAb/control IgG | mAb/control IgG | mAb/control IgG |
| <0.05 | 27 | 12 | 21 | 37 |
| | Immune Infiltrates (mouse) | | | |
| | Raji anti-CD47 | RPMI-8226 anti-CD47 | FaDu anti-CD47 | H1975 anti-CD47 |
| No. of Genes | mAb/ control IgG | mAb/control IgG | mAb/control IgG | mAb/control IgG |
| ≤0.05 | 109 | 59 | 29 | 12 |

Analysis of gene expression in human tumor cells within the four xenograft models was determined using the Human PanCancer IO360 panel (NanoString) and identified 27 genes differentially expressed in Raji tumors treated with anti-CD47 mAb vs control IgG, 12 genes differentially expressed in RPMI-8226 tumors treated with anti-CD47 mAb vs control IgG, 21 genes differentially expressed in FaDu tumors treated with anti-CD47 mAb vs control IgG and 37 genes differentially expressed in H1975 tumors treated with anti-CD47 mAb vs control IgG. Differential regulation is defined as a fold change with an associated p-value of 0.05 or less. For analysis of differential regulation of the murine tumor microenvironment within the same xenograft models, the Mouse Myeloid panel (NanoString) was utilized and identified 109 genes differentially expressed in Raji tumors treated with anti-CD47 mAb vs control IgG, 59 genes differentially expressed in RPMI-8226 tumors treated with anti-CD47 mAb vs control IgG, 29 genes differentially expressed in FaDu tumors treated with anti-CD47 mAb vs control IgG and 12 genes differentially expressed in H1975 tumors treated with anti-CD47 mAb vs control IgG. Individual gene lists for the differentially expressed genes in Table 1 are shown in Table 2 through Table 9. In Table 2 through Table 9, fold changes greater than one signify higher levels of gene expression in the anti-CD47 mAb treated group, while fold changes less than one signify gene expression levels that are higher in control IgG treated group.

TABLE 2
Human tumor specific Genes Differentially Expressed
in anti-CD47 mAb-treated tumors vs control
IgG treated in Raji xenograft model.

| | Raji anti-CD47 | |
|---|---|---|
| Gene Name | mAb/Control IgG | p-value |
| PRR5 | 0.677615572 | 0.002758552 |
| CDKN2B | 0.726339574 | 0.004307869 |
| EGFR | 0.667301798 | 0.00460928 |
| DUSP2 | 0.798262661 | 0.005149109 |
| STAT2 | 1.232901111 | 0.007148975 |
| SPP1 | 0.612621933 | 0.007377117 |
| FAS | 0.810701599 | 0.011435943 |
| ITGA2 | 0.707016691 | 0.013158348 |
| GBP4 | 1.510790861 | 0.013839798 |
| IL4 | 0.621378402 | 0.018415721 |
| BMP2 | 0.620211899 | 0.020515513 |
| DEPTOR | 0.699725428 | 0.021725101 |
| SYK | 1.050248459 | 0.0235816 |
| CCL20 | 0.589479544 | 0.024283039 |
| RPTOR | 1.126291438 | 0.024686531 |
| CCL22 | 0.629883999 | 0.028252311 |
| ICAM5 | 0.680713604 | 0.035318368 |
| WNT3A | 0.541744484 | 0.037421662 |
| CCL21 | 0.638064668 | 0.038429918 |
| E2F3 | 1.095520634 | 0.038903939 |
| ADM | 0.551363348 | 0.039726254 |
| SBNO2 | 0.953420531 | 0.043173079 |
| CD36 | 1.239041244 | 0.043868408 |
| TNFSF13 | 1.35610983 | 0.046231991 |
| BIRC3 | 0.806063579 | 0.047708411 |
| NID2 | 0.79822518 | 0.047990834 |
| CSF2RB | 1.304748652 | 0.050665858 |

TABLE 3
Human tumor specific Genes Differentially Expressed
in anti-CD47 mAb-treated tumors vs controlIgG
treated in RPMI-8226 xenograft model.

| Gene | RPMI-8226 anti-CD47 | |
|---|---|---|
| Name | mAb/control IgG | p-value |
| CD44 | 1.929296559 | 0.005681124 |
| CDKN1A | 1.440257202 | 0.009706505 |
| JAK1 | 1.109967754 | 0.012879116 |
| PTEN | 1.156413848 | 0.017442657 |
| RASGRF1 | 1.652067158 | 0.019479616 |
| CDKN2B | 1.222849696 | 0.021886112 |
| PTCD2 | 1.083441513 | 0.026290502 |
| TREM1 | 1.639458145 | 0.032578572 |
| GNLY | 3.013662406 | 0.036680787 |
| MS4A4A | 1.62603392 | 0.036682696 |
| CD79B | 0.704539047 | 0.037655415 |
| FOXP3 | 1.42655475 | 0.042237146 |

TABLE 4
Human tumor specific Genes Differentially Expressed
in anti-CD47 mAb-treated tumors vs control
IgG treated in FaDu xenograft model.

| | FaDu anti-CD47 | |
|---|---|---|
| Gene Name | mAb/Control IgG | p-value |
| KRAS | 0.945305807 | 0.008618049 |
| PIK3R1 | 1.318963486 | 0.010336151 |
| CX3CL1 | 0.76793765 | 0.019419004 |
| SLC7A5 | 0.914763874 | 0.024568444 |
| MET | 1.063486394 | 0.025281869 |
| ADGRE1 | 0.62962963 | 0.027980981 |
| MGMT | 1.118477989 | 0.033830624 |
| PIK3CG | 1.473232971 | 0.034237132 |
| ACVR1C | 1.580675688 | 0.03512481 |
| CD48 | 0.475419664 | 0.035930062 |
| ELOB | 0.893978173 | 0.037706424 |
| ZEB2 | 1.617660764 | 0.037740217 |
| TRAT1 | 1.669410515 | 0.038768553 |
| CES3 | 0.318423048 | 0.040253489 |
| EPM2AIP1 | 1.109737752 | 0.042525477 |
| FGF18 | 1.647985989 | 0.044330039 |
| CD244 | 2.213973799 | 0.044444849 |
| GHR | 1.290091931 | 0.048369787 |
| LCK | 0.627825213 | 0.048441045 |
| TNFRSF17 | 0.485511912 | 0.048607786 |
| LILRA5 | 0.416292135 | 0.049262512 |

TABLE 5
Human tumor specific Genes Differentially Expressed
in anti-CD47 mAb-treated tumors vs control
IgG treated in H1975 xenograft model.

| | H1975 anti-CD47 | |
|---|---|---|
| Gene Name | mAb/Control IgG | p-value |
| TGFBR1 | 1.137936611 | 4.09848E−05 |
| JAG1 | 1.261621097 | 0.001018476 |
| SLC16A1 | 1.115417718 | 0.002443347 |
| BRD4 | 0.8947418 | 0.003732204 |
| ITGAV | 1.167367276 | 0.007102302 |
| UBE2T | 0.82615784 | 0.007250328 |
| CXCL14 | 1.271076059 | 0.007407232 |
| TNFRSF11B | 1.198703565 | 0.007632686 |
| BMP2 | 1.258626257 | 0.007890741 |
| EPM2AIP1 | 1.057463348 | 0.009467077 |
| P4HA2 | 1.310610677 | 0.011643284 |
| VSIR | 1.394806853 | 0.013519415 |
| ITGA6 | 1.193443726 | 0.014612111 |
| IFNA1 | 1.388991346 | 0.015209411 |
| FGF18 | 1.300627312 | 0.015552054 |
| ITGB8 | 1.298235888 | 0.016063941 |
| TCL1A | 1.390338701 | 0.021523464 |
| IFNGR1 | 1.15869079 | 0.022970034 |
| TLR8 | 0.83926464 | 0.022991707 |
| ROCK1 | 1.155813864 | 0.023758737 |
| CENPF | 0.863395273 | 0.024016842 |
| EGFR | 1.125846434 | 0.024431365 |
| RICTOR | 1.220191943 | 0.024929022 |
| CD44 | 1.157817203 | 0.02870313 |
| PROM1 | 1.261735941 | 0.029433685 |
| FCGR2B | 1.415899745 | 0.032141927 |
| PPARG | 0.81686319 | 0.032813147 |
| PSMB10 | 0.820782117 | 0.035995111 |
| TGFBR2 | 1.116155044 | 0.03643849 |
| ID4 | 1.39235168 | 0.036501902 |
| VHL | 0.918025822 | 0.038732839 |
| COL6A3 | 0.745556752 | 0.040540116 |
| CD276 | 1.140302271 | 0.041092479 |
| DNMT1 | 0.858301546 | 0.043376309 |
| CRABP2 | 0.792583153 | 0.04400308 |
| TWIST1 | 1.274053113 | 0.046405691 |
| PNOC | 1.36796875 | 0.048906986 |

TABLE 6
Genes Differentially Expressed in anti-CD47 mAb-treated tumor immune
infiltrates vs control IgG treated in Raji xenograft model.

| | Raji anti-CD47 | |
|---|---|---|
| Gene Name | mAb/control IgG | p-value |
| Tlr13 | 1.386894519 | 0.000196593 |
| Tnfrsf11a | 1.738745184 | 0.000462011 |
| Nfil3 | 0.563407665 | 0.000967871 |
| Tlr7 | 2.316324627 | 0.001149883 |
| Cd36 | 3.123229256 | 0.001520225 |
| Cytip | 0.538774882 | 0.001804908 |
| Il2 | 0.349816238 | 0.00206972 |
| Lat2 | 1.763487905 | 0.002312436 |
| Adgre1 | 2.17704038 | 0.002517596 |
| Selp | 0.398162085 | 0.002662614 |
| Usp18 | 1.434274809 | 0.003227817 |
| Lamb2 | 0.861617744 | 0.003318548 |
| Ccl9 | 1.485802251 | 0.004001039 |
| Ctsd | 2.413321327 | 0.004082843 |
| Btg2 | 0.446826646 | 0.004981409 |
| Gata2 | 0.695083446 | 0.00534316 |
| Cd68 | 1.87705419 | 0.00551449 |
| Serpine3 | 0.406825829 | 0.005898814 |
| Serpinb6a | 1.220021421 | 0.006060974 |
| Cd180 | 3.631834689 | 0.006098461 |
| Cstb | 1.415333265 | 0.006239047 |
| Fcgr1 | 1.720896452 | 0.006733574 |
| Il1rl1 | 0.515145207 | 0.007850729 |
| Znrf2 | 1.403986721 | 0.007931068 |
| C3ar1 | 2.212696153 | 0.008213475 |
| Vamp2 | 0.682449945 | 0.00838293 |
| Ctss | 1.564003888 | 0.00875824 |
| Grn | 1.796906321 | 0.008939787 |
| Cxcl10 | 2.286114132 | 0.009307906 |
| Cdh5 | 0.80420105 | 0.009470625 |
| C5ar1 | 1.380962226 | 0.0100298 |
| Ier3 | 0.520386246 | 0.010204391 |
| Ctsl | 2.937780379 | 0.010376163 |
| Trem2 | 3.24003861 | 0.010506674 |
| Ccl7 | 1.304243447 | 0.010581587 |
| Tspan7 | 0.674158981 | 0.011310562 |
| Cebpa | 1.557076238 | 0.011576833 |
| Lpl | 6.930772157 | 0.011821518 |
| Cxcl5 | 0.24477741 | 0.011936309 |
| Serpine1 | 0.494163977 | 0.012025389 |
| Vegfa | 0.698803364 | 0.012399403 |
| Csf2 | 0.443839741 | 0.013198354 |
| Eif4ebp2 | 0.778371128 | 0.014341802 |
| Cd84 | 1.618482174 | 0.01467602 |
| Anxa1 | 1.95889214 | 0.014812897 |
| Sema6d | 0.573034295 | 0.014926058 |
| Cd80 | 0.706816349 | 0.015335771 |
| Ptx3 | 0.41013976 | 0.015656188 |
| Il1rn | 1.413837941 | 0.016274857 |
| Nr1h3 | 1.684162922 | 0.016724426 |
| Il1r1 | 0.668470044 | 0.017339315 |
| Plau | 1.743417193 | 0.018400137 |
| Ccr4 | 0.709954522 | 0.01969966 |
| Gsn | 1.492602256 | 0.020707885 |
| Il1b | 0.345221926 | 0.021060262 |
| Stat3 | 0.765423954 | 0.021415589 |
| Sele | 0.526552655 | 0.022526007 |
| Mapk12 | 0.354073498 | 0.023321318 |
| Mmp9 | 1.804029845 | 0.023440832 |
| Hif1a | 0.711299374 | 0.024112437 |
| Col4a1 | 0.700614134 | 0.024169498 |
| Adam8 | 2.54546658 | 0.024774693 |
| Csf3 | 0.480780488 | 0.024822533 |
| Tnfaip6 | 0.457968367 | 0.024889255 |
| Ell2 | 1.710516842 | 0.024966373 |
| Igf1 | 1.843179233 | 0.025008039 |
| Ccl3 | 0.571781293 | 0.025325491 |
| Ski | 0.750538204 | 0.025955056 |
| S100a9 | 0.197349999 | 0.027650726 |
| Rgs16 | 0.645741035 | 0.027711152 |
| Acot11 | 0.519122443 | 0.027731128 |
| Ptafr | 1.310201586 | 0.027957476 |
| Msh2 | 1.355020684 | 0.0294392 |
| Tnfrsf12a | 0.744413666 | 0.029671888 |
| Lipa | 2.815706992 | 0.030489789 |
| Serpinb2 | 0.396036317 | 0.031249253 |
| Ccr1 | 0.596261362 | 0.031523178 |
| Pglyrp1 | 0.604205875 | 0.03172145 |
| Trafd1 | 1.708465795 | 0.031835904 |
| Irf1 | 0.825729716 | 0.032677842 |
| Cxcl14 | 0.517295843 | 0.032728767 |
| Ptgs2 | 0.38234692 | 0.033669292 |
| Marco | 0.524734896 | 0.033775988 |
| Il33 | 0.571318163 | 0.034406798 |
| Tlr8 | 1.796349369 | 0.034551805 |
| Nfkbia | 0.791564763 | 0.035126248 |
| Tnfaip3 | 0.764186982 | 0.035150269 |
| Crem | 0.479092971 | 0.03530193 |
| Anxa4 | 1.520544886 | 0.035922832 |
| Col4a2 | 0.773560379 | 0.036978394 |
| Txn1 | 1.25450488 | 0.037921768 |
| Ccr7 | 0.656068676 | 0.038395749 |
| Nox4 | 0.563827474 | 0.039880952 |
| Tlr5 | 0.607535622 | 0.040452137 |
| H2-D1 | 1.451981622 | 0.040608393 |
| Ets1 | 0.773726667 | 0.041595515 |
| Adamts14 | 0.708304175 | 0.042065795 |
| Cd70 | 1.495203837 | 0.042920572 |
| Adamts1 | 0.638806499 | 0.04442596 |
| Col12a1 | 0.538463793 | 0.045484365 |
| Cebpd | 0.616615067 | 0.046184976 |
| Laptm5 | 1.312672107 | 0.046291915 |
| Cxcr2 | 0.171319959 | 0.046349318 |
| Dhrs3 | 1.564925945 | 0.047257856 |
| Prg3 | 0.450638618 | 0.047282047 |
| Slc16a6 | 0.72008168 | 0.047566473 |
| Vav1 | 1.294372128 | 0.049103783 |
| Nkg7 | 0.476696903 | 0.049659047 |
| Il1rap | 0.67135112 | 0.049681081 |

TABLE 7
Genes Differentially Expressed in anti-CD47
mAb-treated tumor immune infiltrates vs control
IgG treated in RPMI-8226 xenograft model.

| | RPMI-8226 anti-CD47 | |
|---|---|---|
| Gene Name | mAb/control IgG | p-value |
| Ifit1bl2 | 0.318090955 | 0.000372924 |
| Col4a1 | 0.796745348 | 0.004026011 |
| Ctsl | 2.316895704 | 0.004381256 |
| Timd4 | 0.684531469 | 0.004423172 |
| Cd99 | 0.771320011 | 0.005032191 |
| Tlr1 | 1.682910321 | 0.005244514 |
| Fcgr3 | 1.275135515 | 0.00672331 |
| Pglyrp1 | 0.183840504 | 0.006765243 |
| Selplg | 1.185133573 | 0.007081916 |
| Il25 | 0.29245283 | 0.007490333 |
| Kif20a | 0.761019373 | 0.008055402 |
| Man2b1 | 1.386373186 | 0.011534423 |
| Ctsd | 1.204551258 | 0.01408797 |
| Cxcl12 | 0.706634342 | 0.0162312 |
| Gata2 | 0.493154399 | 0.016274141 |
| Cd70 | 0.241877256 | 0.016406349 |
| Adgre1 | 1.756246483 | 0.016749544 |
| Mmp13 | 1.645060229 | 0.01687979 |
| Map3k14 | 0.583633033 | 0.017465335 |
| Col4a2 | 0.772820498 | 0.017615935 |
| Il1rn | 3.44531233 | 0.018451437 |
| Peli1 | 0.783019258 | 0.018879443 |
| Cd69 | 2.271918877 | 0.019449278 |
| Serpinb2 | 0.528205551 | 0.020219997 |
| Daglb | 1.434950887 | 0.020237232 |
| Cdh5 | 0.655575915 | 0.021099117 |
| Fadd | 1.930424528 | 0.022187745 |
| Il1rl1 | 0.638237046 | 0.022305333 |
| Btg2 | 0.59956607 | 0.023491599 |
| Acot3 | 0.51974776 | 0.023537604 |
| Cybb | 1.291565134 | 0.024078228 |
| Csf1r | 1.249650513 | 0.025798752 |
| Alcam | 2.59375 | 0.026499779 |
| Siglec1 | 1.538892762 | 0.027850092 |
| Cav1 | 0.818915334 | 0.028560646 |
| Tlr8 | 1.575182755 | 0.028921201 |
| Ptafr | 1.251588022 | 0.031572899 |
| Cxcl3 | 0.392713801 | 0.032485427 |
| Trem2 | 1.383186016 | 0.033016862 |
| Pggt1b | 0.83920854 | 0.03451755 |
| Top2a | 0.875054945 | 0.034715738 |
| Cd84 | 1.406546666 | 0.037685523 |
| Golim4 | 0.742970918 | 0.038149076 |
| S100a11 | 0.841353214 | 0.038759704 |
| Fut4 | 1.715613776 | 0.038768069 |
| Itgam | 1.553638662 | 0.038883962 |
| Lpl | 2.802654034 | 0.040038446 |
| Fasn | 1.441342496 | 0.04065451 |
| Cd274 | 0.81349797 | 0.040723239 |
| Col11a1 | 1.548266556 | 0.041097092 |
| Fscn1 | 0.748738548 | 0.042948469 |
| Krba1 | 0.737894737 | 0.043487245 |
| Rgs1 | 1.802356262 | 0.043813713 |
| Nox1 | 0.413793103 | 0.046524537 |
| Cdkn1a | 1.384719085 | 0.046572621 |
| Il1r1 | 0.74529739 | 0.046675498 |
| Casp1 | 1.582736092 | 0.047674556 |
| Grn | 1.229931036 | 0.048851552 |
| Casp7 | 1.319331384 | 0.049516972 |
| Havcr1 | 0.445811365 | 0.050786347 |
| Ms4a2 | 0.416461916 | 0.050898953 |

TABLE 8
Genes Differentially Expressed in anti-CD47 mAb-treated tumor immune
infiltrates vs control IgG treated in FaDu xenograft model.

| | FaDu anti-CD47 | |
|---|---|---|
| Gene Name | mAb/control IgG | p-value |
| Adgre1 | 1.516902584 | 0.001731923 |
| Id1 | 0.628838874 | 0.003095236 |
| Trem2 | 1.452868265 | 0.004127761 |
| Csf2ra | 1.480632806 | 0.004527397 |
| Kalrn | 0.587497046 | 0.004828595 |
| Ifit1bl1 | 1.468709855 | 0.005423336 |
| Il1rn | 1.866559722 | 0.005720447 |
| C3ar1 | 1.536660976 | 0.007061782 |
| Il5 | 0.597140039 | 0.00753 |
| Tnfsf10 | 0.620708447 | 0.008142318 |
| Itga4 | 0.654479591 | 0.009551557 |
| Itgam | 1.783651731 | 0.011030274 |
| Ccl26 | 0.241218176 | 0.018091634 |
| Tyrobp | 1.170197993 | 0.021656838 |
| Kcnab1 | 0.403480241 | 0.022491234 |
| Cd180 | 1.902346041 | 0.024907931 |
| Lat2 | 1.587322493 | 0.024996862 |
| Birc5 | 0.646324549 | 0.026688594 |
| Cd40lg | 0.378794179 | 0.028044467 |
| Tlr9 | 1.392219646 | 0.032574155 |
| Sirpa | 1.158096481 | 0.03340137 |
| Stat5a | 1.165007414 | 0.033854352 |
| Laptm5 | 1.24162249 | 0.034135163 |
| Plau | 1.690751293 | 0.034799627 |
| Pik3cg | 1.222535883 | 0.036265352 |
| Il27ra | 0.273972603 | 0.039029377 |
| Lif | 0.636557914 | 0.041543633 |
| Itgax | 1.827801466 | 0.044572726 |
| Prdx1 | 0.826453015 | 0.046714224 |
| Ptgs1 | 0.668704991 | 0.050724337 |
| Myd88 | 1.219682168 | 0.050728298 |

TABLE 9
Genes Differentially Expressed in anti-CD47 mAb-
treated tumor immune infiltrates vs Control (IgG
treated) immune infiltrates in H1975.

| | H1975 anti-CD47 | |
|---|---|---|
| Gene Name | mAb/control IgG | p-value |
| Il1rn | 2.637595993 | 0.00199429 |
| Ccl3 | 1.916071429 | 0.002405839 |
| Ccl4 | 2.091132142 | 0.004452419 |
| Itgam | 2.063473054 | 0.013622859 |
| Il17ra | 1.281697756 | 0.016495838 |
| Sell | 2.327515866 | 0.022150811 |
| Adam8 | 2.024203133 | 0.028365099 |
| Havcr1 | 2.008952551 | 0.030168007 |
| Itgax | 1.793497519 | 0.033842303 |
| Tnfrsf9 | 0.641342756 | 0.038715113 |
| Tek | 1.483068908 | 0.041088044 |
| Cdh1 | 1.531969309 | 0.041210991 |

A comparison of gene expression profiles in responder versus non-responder tumor types was determined in control IgG treated xenograft tumors to understand the baseline gene expression levels in both human tumor specific genes and mouse myeloid genes. Fold changes and p values at baseline as shown in Table 10. Of the human tumor specific genes, 477 genes were differentially expressed in H1975 control IgG vs Raji control IgG treated tumors, 481 genes were differentially expressed in H1975 control IgG vs RPMI-8226 control IgG tumors, 579 genes were differentially expressed in FaDu control IgG vs Raji control IgG treated tumors and 480 genes were differentially expressed in FaDu control IgG vs RPMI-8226 control IgG treated tumors. Mouse immune gene expression profiling identified 392 genes differentially expressed in H1975 control IgG vs Raji control IgG treated tumors, 345 genes differentially expressed in H1975 control IgG vs RPMI-8226 control IgG treated tumors, 203 genes differentially expressed in FaDu control IgG vs Raji control IgG treated tumors and 169 genes differentially expressed in FaDu control IgG vs RPMI-8226 control IgG treated tumors. The total number of differentially expressed genes listed in Table 10 refer to the individual genes listed in Tables 11-18.

TABLE 10
Analysis of NanoString Data Showing A Comparison of Genes
Differentially Expressed at baseline in anti-CD47 responsive
versus nonresponsive xenograft tumor models

| | Tumor Cells (Human) | | | |
|---|---|---|---|---|
| No. of | H1975/Raji | H1975/RPMI- | FaDu/Raji | FaDu/RPMI- |
| Genes | IgG/IgG | 8226 IgG/IgG | IgG/IgG | 8226 IgG/IgG |
| <0.05 | 477 | 481 | 579 | 480 |
| | Immune Infiltrates (mouse) | | | |
| No. of | H1975/Raji | H1975/RPMI- | FaDu/Raji | FaDu/RPMI- |
| Genes | IgG/IgG | 8226 IgG/IgG | IgG/IgG | 8226 IgG/IgG |
| ≤0.05 | 392 | 345 | 203 | 169 |

In Tables 11 through Table 18, fold changes of greater than one are indicative of higher levels of gene expression in the non-responsive tumor types while the fold changes of less than one are indicative of higher levels of gene expression in the responsive tumor types.

TABLE 11
Genes Differentially Expressed in H1975
tumors vs Raji tumors at baseline

| | H1975 control | |
|---|---|---|
| Gene Name | IgG/Raji control IgG | p-value |
| IL10 | 0.064247227 | 1.08863E−07 |
| GPSM3 | 0.18631921 | 1.40907E−07 |
| TMEM173 | 32.22399305 | 1.6462E−07 |
| SOCS1 | 0.110428896 | 4.11857E−07 |
| PVR | 11.65870941 | 7.59934E−07 |
| MET | 13.73049225 | 9.39873E−07 |
| BLK | 0.088840776 | 2.2617E−06 |
| BLM | 0.358552423 | 2.57209E−06 |
| HLA-B | 0.226775895 | 4.66562E−06 |
| PSMB10 | 0.329205513 | 4.94651E−06 |
| IL21R | 0.112570005 | 8.36771E−06 |
| NFKB1 | 0.54272411 | 1.24245E−05 |
| ARID1A | 0.420150327 | 1.37079E−05 |
| IL6R | 3.987362637 | 1.71038E−05 |
| MICB | 0.218617782 | 2.27385E−05 |
| IFITM2 | 6.448103277 | 2.33337E−05 |
| NOTCH2 | 2.414796158 | 2.52748E−05 |
| CD70 | 0.286047422 | 2.9581E−05 |
| ADORA2A | 0.177033273 | 3.49674E−05 |
| CD79A | 0.006901799 | 3.55754E−05 |
| CD276 | 83.21954206 | 3.78161E−05 |
| ATM | 0.322950132 | 4.52737E−05 |
| STAT3 | 1.677156559 | 4.79071E−05 |
| BCL2L1 | 3.249679003 | 5.19146E−05 |
| BMP2 | 4.732783211 | 5.34776E−05 |
| PIK3R1 | 0.33607409 | 5.49373E−05 |
| SYK | 0.051964738 | 5.49933E−05 |
| PRKCA | 4.836098462 | 6.45665E−05 |
| FYN | 0.347824255 | 6.71519E−05 |
| HLA-DQA1 | 0.002067338 | 6.82932E−05 |
| OLFML2B | 3.720754717 | 7.58893E−05 |
| TNFSF12 | 5.416136961 | 8.09063E−05 |
| RELB | 0.318488791 | 8.21783E−05 |
| GBP2 | 4.191726511 | 8.25928E−05 |
| SLAMF7 | 0.180468217 | 8.66606E−05 |
| E2F3 | 0.428953804 | 9.64241E−05 |
| HIF1A | 2.078409751 | 9.76653E−05 |
| TLR9 | 0.099837688 | 0.000100791 |
| IKBKG | 1.842346904 | 0.000103903 |
| DUSP2 | 0.051568448 | 0.000105927 |
| HELLS | 0.40713845 | 0.000110738 |
| NLRC5 | 0.405393626 | 0.000111851 |
| CD247 | 0.229315088 | 0.000114623 |
| TGFB1 | 0.3116074 | 0.000120885 |
| CTSS | 0.197105412 | 0.000122845 |
| TGFBR2 | 2.414945583 | 0.000132148 |
| FADD | 2.705694032 | 0.000150807 |
| FANCA | 0.46732219 | 0.000169303 |
| JAG1 | 230.969942 | 0.000176231 |
| CDKN2B | 7.318334409 | 0.000177056 |
| BRCA2 | 0.387153611 | 0.000183813 |
| ITGB2 | 0.618885909 | 0.000196891 |
| CXCL5 | 5.302150538 | 0.000224135 |
| HMGA1 | 0.533956351 | 0.000229255 |
| CD74 | 0.025437606 | 0.00023564 |
| ITGAV | 16.49266228 | 0.000263137 |
| WDR76 | 0.344432137 | 0.000265044 |
| ITGA6 | 5.501476143 | 0.000272989 |
| ERBB2 | 27.10330189 | 0.000282535 |
| RB1 | 0.081099042 | 0.00028422 |
| CDH1 | 162.60719 | 0.000289496 |
| TNFAIP3 | 0.308242812 | 0.000289661 |
| HLA-DPB1 | 0.008426534 | 0.000292826 |
| ADAM12 | 4.647667176 | 0.000296411 |
| SLC1A5 | 0.438012209 | 0.000340733 |
| PTCD2 | 0.58034584 | 0.000345112 |
| MS4A1 | 0.003674042 | 0.000348867 |
| BBS1 | 3.511094154 | 0.000349652 |
| TNFRSF17 | 0.073673743 | 0.000358632 |
| IL10RA | 0.031607282 | 0.000366388 |
| BIRC3 | 0.179317111 | 0.000368795 |
| RNLS | 16.15215274 | 0.000379694 |
| FCGR2B | 0.013627825 | 0.000385567 |
| HLA-DPA1 | 0.014335202 | 0.000394182 |
| CD79B | 0.004561064 | 0.000396595 |
| TNFRSF1A | 16.5259035 | 0.000408231 |
| BAχ | 0.542088209 | 0.000426673 |
| WNT5A | 0.093009547 | 0.000466165 |
| TLR8 | 4.698829328 | 0.00046791 |
| DNMT1 | 0.430154851 | 0.000476798 |
| MTOR | 0.393759913 | 0.000478984 |
| HLA-DRA | 0.005628373 | 0.0004963 |
| SAMD9 | 6.660249427 | 0.000497544 |
| AKT1 | 1.641574675 | 0.000542388 |
| AXIN1 | 0.524149554 | 0.000552566 |
| TNFSF13 | 3.042967931 | 0.000567602 |
| SFXN1 | 0.547375517 | 0.000569951 |
| IFIT2 | 3.72880771 | 0.000580975 |
| PTPRC | 0.007409066 | 0.000582218 |
| MELK | 1.579469083 | 0.000588373 |
| CTNNB1 | 3.590714769 | 0.000592802 |
| RAD51 | 0.526274254 | 0.000615574 |
| MSH2 | 0.582166119 | 0.000617472 |
| NFIL3 | 2.912486253 | 0.000622563 |
| LY9 | 0.064013745 | 0.000651157 |
| CD274 | 5.155667603 | 0.000669381 |
| EGFR | 41.33915997 | 0.000711285 |
| TYMS | 0.417278179 | 0.000716061 |
| ITGA4 | 0.008813041 | 0.000719313 |
| CD80 | 0.022699319 | 0.000757057 |
| BCL2 | 0.055314443 | 0.00077395 |
| HLA-DMB | 0.021740361 | 0.00082223 |
| JAK3 | 3.032564689 | 0.000836727 |
| IL15 | 2.395762413 | 0.000879062 |
| CDK2 | 1.351093279 | 0.000890169 |
| UBA7 | 1.931672084 | 0.000897817 |
| BRD4 | 0.684242511 | 0.00090426 |
| CXCL3 | 7.536600369 | 0.00091511 |
| PC | 12.76055237 | 0.000927814 |
| CD68 | 6.675104061 | 0.000961007 |
| WNT7B | 17.55143639 | 0.00098981 |
| IL1A | 7.834444244 | 0.000991437 |
| CD38 | 0.010309973 | 0.001017801 |
| CX3CL1 | 145.1279923 | 0.001032085 |
| CASP9 | 0.256272289 | 0.001045133 |
| SERPINH1 | 39.62176822 | 0.001051881 |
| FZD9 | 2.825074902 | 0.001112356 |
| TNF | 0.120288864 | 0.001118597 |
| GLUL | 1.780116478 | 0.001120635 |
| CD27 | 0.00349194 | 0.001140725 |
| RPS6KB1 | 0.506571055 | 0.001148605 |
| NF1 | 1.85580467 | 0.001154662 |
| KIF2C | 0.501748305 | 0.001207165 |
| CD48 | 0.002471856 | 0.001207378 |
| FAM30A | 0.064544004 | 0.001209751 |
| SLC16A1 | 0.64794632 | 0.001218985 |
| FCGR2A | 0.235100142 | 0.001220798 |
| TNFRSF10C | 3.976013326 | 0.001232714 |
| HLA-DOB | 0.025677973 | 0.001268152 |
| PARP12 | 4.173124162 | 0.00126925 |
| PFKM | 0.508700745 | 0.001279561 |
| NFKB2 | 0.413914298 | 0.001313153 |
| CCND1 | 85.55598777 | 0.001330696 |
| TNFSF9 | 0.530799153 | 0.001332703 |
| CD44 | 146.8425377 | 0.001337102 |
| HLA-DOA | 0.075399977 | 0.001356966 |
| IRF1 | 0.516165745 | 0.001386181 |
| INHBA | 4.23355526 | 0.001412248 |
| MAP3K8 | 0.142353679 | 0.001450547 |
| PKM | 1.56229795 | 0.00148196 |
| NFKBIE | 0.09398409 | 0.001511226 |
| MAP3K5 | 0.581443162 | 0.001513395 |
| HLA-DQB1 | 0.015842949 | 0.001526247 |
| ANLN | 6.267534171 | 0.001547349 |
| TPI1 | 0.575618197 | 0.001553029 |
| B2M | 0.435467305 | 0.001563477 |
| PPARG | 44.46174938 | 0.001564774 |
| RUNX3 | 0.009520033 | 0.0015729 |
| GOT2 | 0.61156119 | 0.001600418 |
| CBLC | 2.446431266 | 0.001609928 |
| TNFRSF9 | 2.22405514 | 0.00162748 |
| RIPK1 | 2.266703423 | 0.001681431 |
| BCAT1 | 0.072710777 | 0.001709434 |
| TNFRSF10B | 2.286148214 | 0.001749594 |
| CYBB | 0.016033622 | 0.001774466 |
| IRF8 | 0.004644557 | 0.001794356 |
| PTGER4 | 0.443091043 | 0.001825685 |
| GPC4 | 8.707317073 | 0.00185254 |
| MB21D1 | 8.562225141 | 0.001902966 |
| NFATC2 | 0.258770183 | 0.001921934 |
| LRRC32 | 0.408236208 | 0.00192405 |
| IL16 | 0.028133257 | 0.002006552 |
| PIK3CD | 0.06423217 | 0.002009134 |
| IL18 | 70.55306334 | 0.002011975 |
| TNFSF8 | 0.619117075 | 0.002074946 |
| RAD50 | 0.660263811 | 0.002124755 |
| GLS | 1.959107311 | 0.002135424 |
| FUT4 | 1.806022528 | 0.002139729 |
| EPCAM | 107.4719028 | 0.002151117 |
| TTC30A | 4.061503583 | 0.002187756 |
| P4HA2 | 12.95057412 | 0.002213985 |
| CCNB1 | 2.49564669 | 0.002230129 |
| HLA-DRB1 | 0.003700872 | 0.002238469 |
| TGFB3 | 2.734609878 | 0.002296374 |
| TPM1 | 82.22792512 | 0.002341266 |
| PIK3R2 | 1.561571797 | 0.002344301 |
| SRP54 | 0.619433184 | 0.002360253 |
| ITGB8 | 56.84642321 | 0.002373051 |
| CHUK | 0.593001574 | 0.00240504 |
| THBS1 | 1196.708558 | 0.002448459 |
| CXCL6 | 15.92584515 | 0.002474643 |
| ITGB3 | 10.81063038 | 0.00247494 |
| PNOC | 0.171558772 | 0.002483755 |
| UBE2C | 2.142167931 | 0.002502134 |
| HDAC11 | 3.093630623 | 0.002512996 |
| IGF2R | 1.872748289 | 0.002525163 |
| EPM2AIP1 | 0.424107115 | 0.002540939 |
| CCL20 | 2.27664904 | 0.002541064 |
| SREBF1 | 0.503634678 | 0.002567364 |
| MX1 | 32.53668962 | 0.002585735 |
| GHR | 0.695072938 | 0.0026142 |
| PECAM1 | 0.387404918 | 0.002629385 |
| BRD3 | 1.451931918 | 0.002633368 |
| IER3 | 14.38548851 | 0.002705529 |
| LIF | 73.22141503 | 0.002767509 |
| CRABP2 | 14.16795512 | 0.002770806 |
| FSTL3 | 64.47832817 | 0.002810696 |
| POLD1 | 0.509184069 | 0.002824889 |
| CD84 | 0.007046394 | 0.002834189 |
| CSF2RB | 0.315359193 | 0.002871291 |
| HLA-DMA | 0.066642422 | 0.002883052 |
| ULBP2 | 2.343249769 | 0.002888014 |
| NRAS | 0.633766378 | 0.002907965 |
| VCAN | 173.3314917 | 0.002984638 |
| CCND3 | 0.255459014 | 0.002991093 |
| IL2RG | 0.006802443 | 0.003047652 |
| SERPINA1 | 73.88257669 | 0.00310404 |
| HLA-F | 0.274136397 | 0.003140277 |
| PPARGC1B | 0.313613604 | 0.003167086 |
| PLA1A | 0.408092265 | 0.003203263 |
| F2RL1 | 129.3762322 | 0.00321498 |
| NT5E | 63.88647978 | 0.003251886 |
| LY96 | 0.634269135 | 0.003283164 |
| TCF3 | 0.249178438 | 0.003290159 |
| LAMB3 | 867.9981982 | 0.003331768 |
| CASP3 | 0.628646469 | 0.003339098 |
| ZEB2 | 0.100260796 | 0.003410348 |
| TLR1 | 0.068618193 | 0.003434149 |
| CLECL1 | 0.002487847 | 0.003435571 |
| HLA-A | 0.25122443 | 0.00348434 |
| CXCR4 | 0.004312055 | 0.003491058 |
| ENTPD1 | 0.252270276 | 0.003549554 |
| DPP4 | 11.23091664 | 0.003571619 |
| DTX4 | 0.696608058 | 0.003629717 |
| FLNB | 2.90574418 | 0.003708921 |
| AREG | 53.67951374 | 0.003808212 |
| PTGS2 | 3.0257934 | 0.003867853 |
| TGFB2 | 26.01107941 | 0.003901518 |
| TNFRSF14 | 0.235095987 | 0.003926428 |
| ICOSLG | 0.098664572 | 0.003949581 |
| SPIB | 0.012312691 | 0.003977924 |
| PSMB9 | 0.542804779 | 0.003997444 |
| MFGE8 | 5.531830143 | 0.004017744 |
| CD40 | 0.221024323 | 0.004083005 |
| ICAM5 | 0.379340724 | 0.004101867 |
| COL6A3 | 6.064094713 | 0.004141196 |
| CCL4 | 0.021748277 | 0.004293687 |
| SPRY4 | 14.86439056 | 0.004312848 |
| HLA-E | 0.535868147 | 0.004332753 |
| TCL1A | 0.001628589 | 0.004361385 |
| EZH2 | 0.608110698 | 0.004371233 |
| FAS | 0.519293961 | 0.004425647 |
| FGF13 | 6.842942865 | 0.004433692 |
| CD86 | 0.026847993 | 0.004509713 |
| IL1B | 2.032588296 | 0.004640264 |
| CENPF | 1.254880239 | 0.004826115 |
| MFNG | 0.062327358 | 0.00486148 |
| BAD | 1.49123196 | 0.004883971 |
| HMGB1 | 0.441680361 | 0.004939736 |
| COL5A1 | 170.4327974 | 0.004977122 |
| VCAM1 | 0.390465598 | 0.005094735 |
| STAT2 | 0.638925729 | 0.005111077 |
| CDKN2A | 0.59220164 | 0.005133517 |
| IRF4 | 0.049196781 | 0.005267268 |
| MAP3K7 | 1.279129196 | 0.005302277 |
| CDK6 | 0.737737099 | 0.005343334 |
| BATF3 | 1.714041746 | 0.005385308 |
| NECTIN2 | 19.63117927 | 0.005423287 |
| ID4 | 3.51861167 | 0.005456416 |
| HLA-DQA2 | 0.337824389 | 0.005554444 |
| ICAM2 | 0.330420588 | 0.005608801 |
| CD45RA | 0.004241374 | 0.005624592 |
| TLR2 | 1.831025914 | 0.005770327 |
| PRKACB | 0.401685272 | 0.005801513 |
| FOSL1 | 118.8455443 | 0.005856825 |
| TRAF1 | 0.163320147 | 0.006039641 |
| IL11 | 68.90015426 | 0.006049442 |
| EGF | 4.384847912 | 0.006073425 |
| RPL7A | 0.46359214 | 0.006113644 |
| TAF3 | 0.647574367 | 0.006128286 |
| FCRL2 | 0.032742626 | 0.006135037 |
| GPR160 | 0.046994286 | 0.006215277 |
| PGPEP1 | 0.502590177 | 0.006271815 |
| PDGFB | 3.864312456 | 0.006361748 |
| RBL2 | 0.832430503 | 0.006382578 |
| IRF3 | 0.610248044 | 0.006389093 |
| HLA-C | 0.097771935 | 0.006454391 |
| MICA | 3.442659385 | 0.006543038 |
| RPL23 | 0.592266696 | 0.006571502 |
| IFITM1 | 5.15592452 | 0.006657696 |
| EIF4EBP1 | 22.65167782 | 0.006680186 |
| CD19 | 0.013102376 | 0.00675508 |
| IL6 | 10.36851598 | 0.006763891 |
| ITGAL | 0.094974835 | 0.006791481 |
| TAP2 | 0.439357974 | 0.007050284 |
| KAT2B | 0.335760903 | 0.007063783 |
| OASL | 3.329795261 | 0.007075781 |
| SLC2A1 | 0.587002824 | 0.007113218 |
| NOTCH1 | 2.171742656 | 0.007124646 |
| IL24 | 3.752304553 | 0.007136092 |
| MLH1 | 0.658977931 | 0.007170484 |
| CCL22 | 0.034336418 | 0.007279645 |
| PDCD1LG2 | 2.590412713 | 0.007280357 |
| WNT5B | 0.553675992 | 0.007322666 |
| ITGAX | 0.760481552 | 0.007479185 |
| CD69 | 0.022629391 | 0.007531482 |
| JAG2 | 2.989288447 | 0.007606909 |
| MMP1 | 145.2622278 | 0.007736469 |
| ITGA2 | 101.7373197 | 0.007769495 |
| HES1 | 2.77584002 | 0.007804922 |
| OAS3 | 50.80061983 | 0.007912305 |
| SAMSN1 | 0.07548424 | 0.008043547 |
| CCNO | 2.054995871 | 0.008303319 |
| TIGIT | 0.675901964 | 0.008332228 |
| WNT10A | 0.118190612 | 0.008345997 |
| ARG2 | 0.372977442 | 0.008405387 |
| EIF2AK2 | 1.562041265 | 0.008416798 |
| EGR1 | 1.271753764 | 0.008579928 |
| TNFRSF4 | 0.529488946 | 0.008678128 |
| IFIH1 | 0.414566178 | 0.008694829 |
| TLR5 | 6.619548407 | 0.00877541 |
| NECTIN1 | 2.887224329 | 0.009026663 |
| SIRPA | 19.34279919 | 0.00915566 |
| DKK1 | 68.37088916 | 0.009263989 |
| PMS2 | 0.791342395 | 0.00934818 |
| HERC6 | 7.658971668 | 0.009518071 |
| SGK1 | 0.395044381 | 0.009655662 |
| LCK | 0.332651055 | 0.009697062 |
| SERPINB5 | 55.17384106 | 0.009767486 |
| MAPK10 | 0.523194788 | 0.009897491 |
| GNG4 | 0.587483499 | 0.010093019 |
| VEGFA | 7.206627365 | 0.010134841 |
| CDKN1C | 12.55731407 | 0.010194596 |
| LYZ | 0.557335634 | 0.010276047 |
| STC1 | 15.85526485 | 0.010354726 |
| PDGFA | 6.241770276 | 0.010464683 |
| FCN1 | 0.526361656 | 0.010470641 |
| CSF2 | 8.350981075 | 0.010545012 |
| PDK1 | 0.477366507 | 0.010575723 |
| ZEB1 | 0.283131397 | 0.010633554 |
| CEBPB | 4.803264721 | 0.010896203 |
| LTB | 0.061018033 | 0.010974196 |
| TNFRSF11A | 3.881588647 | 0.011032937 |
| KRAS | 0.475362141 | 0.011149881 |
| CCL3/L1 | 0.003284886 | 0.011195868 |
| CXCL2 | 75.92846034 | 0.011372312 |
| CSF1R | 4.651190981 | 0.011436906 |
| SMAP1 | 0.449913696 | 0.011451715 |
| PCK2 | 0.339488049 | 0.011589775 |
| PSMB8 | 0.449351426 | 0.011658419 |
| CD58 | 0.486697396 | 0.01169 |
| PLOD2 | 259.8772874 | 0.012052785 |
| VHL | 0.468190207 | 0.012100635 |
| TRAT1 | 0.606895441 | 0.012220998 |
| KLRB1 | 0.591067864 | 0.01231171 |
| BCL6B | 1.622628062 | 0.012543445 |
| JAK1 | 0.589057692 | 0.012569558 |
| TLR7 | 0.217908058 | 0.012693836 |
| TNFRSF11B | 1.302272727 | 0.01285953 |
| COL11A1 | 0.555509077 | 0.01286051 |
| LOXL2 | 2.394563348 | 0.012970668 |
| PVRIG | 0.205756339 | 0.013270423 |
| CD3E | 0.633715685 | 0.013281349 |
| GOT1 | 1.451509907 | 0.013372148 |
| KDR | 2.592203898 | 0.013385983 |
| CXCL8 | 31.50008685 | 0.01345515 |
| PTEN | 0.754631591 | 0.013568663 |
| TAPBPL | 1.256399858 | 0.01390406 |
| IFI16 | 0.858215284 | 0.0139126 |
| S100A9 | 64.77697842 | 0.013927048 |
| SOX2 | 5.447427681 | 0.01434819 |
| TNFRSF25 | 2.750121714 | 0.014440957 |
| MGMT | 1.581009481 | 0.014523021 |
| TMEM140 | 0.402984256 | 0.014527114 |
| EIF2B4 | 0.834304633 | 0.01457338 |
| VTCN1 | 1.230887277 | 0.014812732 |
| GLUD1 | 0.830571113 | 0.01496018 |
| COL17A1 | 57.13199879 | 0.015042499 |
| ADM | 10.07905305 | 0.015312289 |
| IFI27 | 19.09793421 | 0.015734544 |
| CD45RO | 0.488169181 | 0.015749084 |
| CXCL1 | 25.48546424 | 0.015761186 |
| LDHB | 0.305015002 | 0.015869504 |
| LILRA1 | 0.537882281 | 0.016007229 |
| IFNAR1 | 0.880378079 | 0.016135217 |
| BAMBI | 1.33557047 | 0.016277647 |
| UBE2T | 0.531452413 | 0.016531119 |
| AXL | 12.38279362 | 0.016815238 |
| HK1 | 1.888526847 | 0.016822211 |
| FCGR3A/B | 0.66989635 | 0.016856033 |
| IRF2 | 0.522265692 | 0.017626229 |
| RASGRF1 | 0.429351054 | 0.017839735 |
| IRF9 | 1.541256737 | 0.017917174 |
| CCNA1 | 12.51348376 | 0.018572726 |
| APH1B | 9.189145534 | 0.018927793 |
| TREM1 | 2.720595353 | 0.018934956 |
| BID | 0.574122016 | 0.018990196 |
| ALDOC | 0.645764668 | 0.019043831 |
| TYMP | 6.998817459 | 0.019053384 |
| NFKBIA | 0.833475681 | 0.019089549 |
| TWF1 | 4.003088119 | 0.01951246 |
| LAMC2 | 115.74743 | 0.019784357 |
| BRIP1 | 1.603301663 | 0.019840314 |
| SNAI1 | 4.970481564 | 0.019873312 |
| SBNO2 | 1.963544844 | 0.020062473 |
| MSH6 | 0.650284748 | 0.020110113 |
| ROCK1 | 0.579187783 | 0.020352255 |
| ANGPTL4 | 53.97861451 | 0.020613267 |
| GZMK | 0.549312977 | 0.02062562 |
| CES3 | 0.190385208 | 0.020694532 |
| HAVCR2 | 0.295832478 | 0.020720742 |
| EDN1 | 6.98929263 | 0.02085412 |
| ITPK1 | 1.304735061 | 0.020894815 |
| FGF18 | 0.713040486 | 0.021067953 |
| APOL6 | 1.329828995 | 0.021120976 |
| ALDOA | 1.533239472 | 0.021317819 |
| NKG7 | 0.138409488 | 0.021525365 |
| NEIL1 | 0.347271927 | 0.021635944 |
| TNFSF4 | 0.435732517 | 0.021988898 |
| DUSP1 | 4.598522109 | 0.022233537 |
| IL2RB | 0.531258591 | 0.02242709 |
| CD2 | 0.38750676 | 0.022833692 |
| BNIP3 | 2.650540227 | 0.023381549 |
| CCNE1 | 1.264003761 | 0.0238245 |
| TAP1 | 0.388511133 | 0.024065403 |
| TAPBP | 0.723770868 | 0.024178337 |
| DUSP5 | 1.861214886 | 0.024189775 |
| CXCL9 | 0.336707369 | 0.024199304 |
| CD14 | 3.954636182 | 0.024634366 |
| MKI67 | 0.766752822 | 0.024763557 |
| CASP1 | 0.545159474 | 0.024966466 |
| FBP1 | 20.20157266 | 0.025124866 |
| JAK2 | 0.579262274 | 0.026210023 |
| ATF3 | 1.329028841 | 0.026452687 |
| MYD88 | 0.766656615 | 0.026599018 |
| IL17A | 0.628462501 | 0.026601718 |
| STAT4 | 3.220178042 | 0.026748469 |
| IFNGR1 | 0.549364113 | 0.026825671 |
| SIRPB2 | 2.858319605 | 0.027390941 |
| TNFRSF10D | 3.16428639 | 0.027405144 |
| ENO1 | 0.758744422 | 0.02741494 |
| MRE11 | 0.769350467 | 0.027857349 |
| TNFRSF1B | 0.089529164 | 0.02815507 |
| MS4A4A | 0.657798686 | 0.028245285 |
| PLA2G2A | 0.764517948 | 0.02883439 |
| S100A8 | 1.588235294 | 0.028994615 |
| LILRB2 | 0.560880911 | 0.029489869 |
| KIR2DL3 | 0.750568137 | 0.029606353 |
| CXCL10 | 0.030171343 | 0.03010874 |
| BRCA1 | 0.782570832 | 0.030148935 |
| FAP | 2.400961788 | 0.031589486 |
| CXCR2 | 0.568520812 | 0.031818111 |
| NBN | 0.77715025 | 0.032286523 |
| IFNGR2 | 0.764670838 | 0.032442966 |
| OAS2 | 0.262921572 | 0.032492355 |
| MAP3K12 | 2.074060759 | 0.033516884 |
| TNKS | 0.828911465 | 0.033894612 |
| IL1R2 | 0.10447363 | 0.033931858 |
| PTPN11 | 0.761711868 | 0.034478143 |
| CCL5 | 0.468681238 | 0.03450911 |
| BTLA | 0.227785783 | 0.034951377 |
| SLC7A5 | 1.397225391 | 0.034976996 |
| TNFSF13B | 0.613440351 | 0.036208548 |
| PRR5 | 1.118449944 | 0.036219468 |
| FCGRT | 1.488051471 | 0.036395134 |
| CD28 | 0.581772899 | 0.03678948 |
| BBC3 | 0.542704185 | 0.037311243 |
| IL32 | 5.071346305 | 0.038444327 |
| EIF5AL1 | 0.752221865 | 0.038819245 |
| TNFAIP6 | 1.573868488 | 0.039017842 |
| CD8B | 0.562542587 | 0.039151743 |
| CCL19 | 0.688959144 | 0.039733848 |
| ITGAM | 0.601693678 | 0.041463203 |
| CD3G | 0.792331136 | 0.041551046 |
| SIGLEC1 | 0.487555639 | 0.041645039 |
| RIPK2 | 0.823861636 | 0.041738518 |
| CDC20 | 1.342629552 | 0.041797494 |
| ELOB | 0.620940074 | 0.043074949 |
| RIPK3 | 0.63921145 | 0.043646712 |
| CDH11 | 1.458402976 | 0.045070439 |
| PIAS4 | 1.116051976 | 0.045471516 |
| PIK3CG | 0.540949175 | 0.045850423 |
| ERO1A | 3.097683906 | 0.045930837 |
| SH2D1A | 0.501818182 | 0.047333446 |
| ICOS | 0.514253486 | 0.04937549 |
| TBXAS1 | 0.582360949 | 0.049954771 |
| APOE | 5.231221424 | 0.050679378 |

TABLE 12
Genes Differentially Expressed in H1975
tumors vs RPMI-8226 tumors at baseline

| | H1975 control IgG/ | |
|---|---|---|
| Gene Name | RPMI-8226 control IgG | p-value |
| JAK3 | 0.252873226 | 7.4456E−08 |
| CD276 | 7.723077622 | 7.47796E−08 |
| GPR160 | 0.009018245 | 7.57167E−08 |
| RB1 | 0.132868223 | 1.75745E−07 |
| PVR | 7.759600269 | 3.16286E−07 |
| RUNX3 | 0.015132184 | 3.56054E−07 |
| HLA-B | 0.116474559 | 3.70035E−07 |
| MB21D1 | 0.211491555 | 7.08466E−07 |
| HLA-A | 0.095532019 | 1.56217E−06 |
| HIF1A | 9.669484491 | 2.4266E−06 |
| CDH1 | 9.048332957 | 3.70615E−06 |
| CTSS | 0.189194068 | 4.30592E−06 |
| HLA-C | 0.052725093 | 4.88961E−06 |
| TGFBR1 | 8.02145524 | 7.73252E−06 |
| RNLS | 8.282617383 | 1.27821E−05 |
| B2M | 0.24762143 | 1.28686E−05 |
| GLUD1 | 2.060185776 | 1.93144E−05 |
| CDH11 | 2.548426606 | 2.25627E−05 |
| UBA7 | 5.113575526 | 2.30093E−05 |
| CD86 | 0.058189327 | 2.3058E−05 |
| MET | 68.09869959 | 2.35967E−05 |
| CX3CR1 | 0.002802084 | 2.54618E−05 |
| RPL7A | 0.559644463 | 2.62686E−05 |
| ULBP2 | 5.194885012 | 3.36285E−05 |
| BMP2 | 3.642257938 | 3.62102E−05 |
| PSMB10 | 0.436507821 | 3.63242E−05 |
| DTX3L | 1.510576414 | 5.71015E−05 |
| MICB | 0.122708006 | 5.82436E−05 |
| LTBP1 | 4.329163771 | 5.84341E−05 |
| BCL2L1 | 2.182945791 | 6.07635E−05 |
| FUT4 | 2.591536712 | 6.58372E−05 |
| MAGEA1 | 0.060890362 | 6.65001E−05 |
| SERPINH1 | 3.951603015 | 7.27455E−05 |
| ITGB2 | 4.981244109 | 7.63871E−05 |
| PRKCA | 22.54070501 | 8.88649E−05 |
| TNFSF12 | 8.261797027 | 9.66451E−05 |
| VHL | 0.626934801 | 9.90039E−05 |
| POLD1 | 0.395702199 | 0.000101372 |
| TAP1 | 0.389477414 | 0.000112855 |
| MGMT | 1.820648683 | 0.000113397 |
| TGFBR2 | 5.495834379 | 0.000115421 |
| ATM | 0.260430926 | 0.00013701 |
| IL1A | 6.053591314 | 0.000138267 |
| ICAM3 | 0.126964944 | 0.000139948 |
| SMAP1 | 0.325409817 | 0.000157928 |
| JAG1 | 18.93641481 | 0.000167614 |
| ICOSLG | 0.095232679 | 0.000187007 |
| RASAL1 | 0.166254421 | 0.000194621 |
| LGALS9 | 0.162568404 | 0.000208321 |
| CDKN2B | 12.31114248 | 0.000211153 |
| ITPK1 | 1.596549279 | 0.000218837 |
| MFNG | 0.107682773 | 0.000232631 |
| BCL2 | 0.034597765 | 0.000250091 |
| TLR7 | 0.253329038 | 0.000258926 |
| H2AFX | 2.425493692 | 0.000269669 |
| ITGAE | 2.195203936 | 0.000282276 |
| TMEM173 | 2.465341925 | 0.00029681 |
| PRKACB | 0.350011661 | 0.000340992 |
| BBS1 | 1.960986804 | 0.000344521 |
| MYC | 0.442192526 | 0.000349687 |
| MAML2 | 0.401118635 | 0.000363835 |
| IFI16 | 0.649012329 | 0.000369349 |
| TAPBP | 0.422477436 | 0.000369592 |
| TMEM140 | 0.300185931 | 0.000369601 |
| ADAM12 | 8.405313496 | 0.000372232 |
| E2F3 | 0.292774504 | 0.000393777 |
| TYMS | 0.455559677 | 0.000398542 |
| GLS | 2.807285584 | 0.000409613 |
| TNFRSF1A | 2.506129202 | 0.000409826 |
| FADD | 2.632284683 | 0.000410337 |
| TLR8 | 7.198602983 | 0.000417714 |
| ITGAV | 4.609252401 | 0.000470321 |
| ZEB1 | 0.437357684 | 0.000497392 |
| PIK3CA | 0.443578845 | 0.000506032 |
| EGR1 | 4.56747089 | 0.000508093 |
| CXCL6 | 3.546955959 | 0.00051614 |
| IFITM2 | 5.72616318 | 0.0005201 |
| IFIH1 | 0.479467237 | 0.000533499 |
| NOTCH1 | 0.492329691 | 0.000535707 |
| ZEB2 | 2.113275517 | 0.000556452 |
| SLC16A1 | 1.192167449 | 0.000558925 |
| CXCL5 | 10.27903514 | 0.00057151 |
| ERBB2 | 47.14584615 | 0.000592211 |
| RIPK2 | 2.61676715 | 0.000602229 |
| EGFR | 46.83294228 | 0.000608191 |
| KAT2B | 0.257568371 | 0.000609016 |
| CXCL12 | 0.039241516 | 0.000633231 |
| CBLC | 5.509770603 | 0.000639266 |
| CCL20 | 5.515846258 | 0.000643387 |
| MAP3K7 | 1.367684331 | 0.000645858 |
| PTPRC | 0.075575729 | 0.000670935 |
| PTCD2 | 0.59834909 | 0.000674434 |
| BLK | 0.115363706 | 0.000681298 |
| TNFRSF9 | 10.84847036 | 0.00070466 |
| HLA-DMA | 0.588922787 | 0.000721414 |
| CXCL3 | 4.564828614 | 0.000723526 |
| ITGA4 | 0.025816881 | 0.000724543 |
| KIF2C | 0.400220644 | 0.000764315 |
| CD274 | 11.53162027 | 0.000786366 |
| TYMP | 0.195945382 | 0.000816948 |
| NFKBIE | 0.418721813 | 0.000822669 |
| IFIT3 | 2.303119057 | 0.000839923 |
| GPSM3 | 0.520683192 | 0.000892364 |
| HLA-DMB | 0.33932516 | 0.000906881 |
| INHBA | 6.687841817 | 0.000948569 |
| CD74 | 0.12001492 | 0.000963425 |
| TNFRSF1B | 0.040625972 | 0.000979249 |
| CASP1 | 0.314841842 | 0.000980187 |
| PIK3R1 | 0.366203089 | 0.000990617 |
| APC | 0.773756855 | 0.000993352 |
| TAP2 | 0.47699095 | 0.001031524 |
| CX3CL1 | 204.3532584 | 0.001037196 |
| WNT7B | 20.50847187 | 0.001045571 |
| UBE2C | 50.78876768 | 0.001054948 |
| NFIL3 | 1.98787095 | 0.00106373 |
| LDHB | 0.589071731 | 0.001072683 |
| AKT1 | 3.133783918 | 0.001118715 |
| DNMT1 | 0.701836949 | 0.001131867 |
| IRF2 | 0.560699002 | 0.001172767 |
| HLA-F | 0.277616864 | 0.001195294 |
| PDCD1 | 0.205481805 | 0.001196974 |
| PC | 15.98400783 | 0.001216942 |
| ITGA6 | 122.4508004 | 0.001236655 |
| TTC30A | 2.94282535 | 0.00127205 |
| CD19 | 2.561253197 | 0.001277427 |
| CXCR4 | 0.008213047 | 0.00128808 |
| WNT2B | 6.201217193 | 0.001288202 |
| MELK | 2.15871624 | 0.001289499 |
| PSMB8 | 0.692632245 | 0.001290144 |
| LCK | 3.933598726 | 0.001293175 |
| CD44 | 138.0697152 | 0.001336592 |
| OAS2 | 0.316839005 | 0.001368175 |
| ADORA2A | 0.243972918 | 0.001386381 |
| APOL6 | 1.452634118 | 0.001411111 |
| NOD2 | 0.180893028 | 0.001416035 |
| TNFRSF10B | 4.327809165 | 0.001468882 |
| CDK6 | 0.581426084 | 0.001482444 |
| HDAC5 | 0.422212553 | 0.00148574 |
| PECAM1 | 0.332388951 | 0.001501372 |
| P4HA2 | 7.931500506 | 0.001504217 |
| CCND2 | 0.001209972 | 0.00151175 |
| PPARG | 163.789899 | 0.001564689 |
| SOX11 | 0.227889579 | 0.001581338 |
| CD8B | 0.001686788 | 0.001642896 |
| KRAS | 0.290565906 | 0.00166711 |
| TNFRSF25 | 0.23785212 | 0.001687667 |
| SYK | 0.151013844 | 0.001693354 |
| CCND1 | 6.787673045 | 0.001695416 |
| RELB | 1.5285555 | 0.001718516 |
| IFIT2 | 16.74651246 | 0.001742766 |
| TLR1 | 0.067239678 | 0.001805581 |
| FGFR1 | 2.163837527 | 0.001811809 |
| TGFB3 | 4.398675878 | 0.001828763 |
| ARG2 | 0.204694808 | 0.001843852 |
| NOTCH2 | 0.603713947 | 0.001866162 |
| NF1 | 1.558727437 | 0.001871464 |
| IL16 | 0.043057594 | 0.001970479 |
| CD38 | 0.007983032 | 0.001995922 |
| IRF4 | 0.008753288 | 0.002019732 |
| IL18 | 9.034439657 | 0.002027493 |
| TNFRSF10C | 7.944086976 | 0.002033727 |
| TLR4 | 0.036548656 | 0.002038105 |
| TNFRSF18 | 0.113648694 | 0.002076276 |
| SLC2A1 | 0.491713583 | 0.002093392 |
| ITGB3 | 24.15490534 | 0.002094685 |
| ARID1A | 0.408418949 | 0.002143812 |
| DTX4 | 2.122679717 | 0.00221565 |
| ANLN | 7.010987226 | 0.002237269 |
| ITGA1 | 3.243343518 | 0.002243106 |
| GPC4 | 11.3626943 | 0.002251753 |
| ITGB8 | 52.944246 | 0.002308907 |
| CD68 | 1.912952366 | 0.002323512 |
| IRF5 | 0.446625658 | 0.002341754 |
| VEGFB | 0.506072299 | 0.002368154 |
| TLR2 | 4.529527991 | 0.002382281 |
| CASP3 | 0.646912969 | 0.002444544 |
| THBS1 | 1356.372543 | 0.002447321 |
| MTOR | 0.573416921 | 0.002449403 |
| VTCN1 | 2.185804534 | 0.002483811 |
| FSTL3 | 77.45072518 | 0.002554974 |
| TPM1 | 9.818579785 | 0.002594172 |
| TNFRSF17 | 0.011966124 | 0.002640315 |
| PFKM | 0.599897837 | 0.002661637 |
| BLM | 0.327165241 | 0.002665978 |
| CCR2 | 0.026121101 | 0.002677543 |
| BID | 1.434274685 | 0.002678175 |
| IER3 | 11.96999223 | 0.002709648 |
| DPP4 | 12.09423904 | 0.002723828 |
| FANCA | 1.555039274 | 0.002735619 |
| STAT4 | 0.127281257 | 0.002737905 |
| LIF | 112.6428571 | 0.002746627 |
| CTNNB1 | 5.276078965 | 0.002801298 |
| CDKN2A | 0.544404935 | 0.002811567 |
| IL10 | 0.094422955 | 0.002812246 |
| HES1 | 11.48285877 | 0.002831231 |
| JAG2 | 2.787230248 | 0.002885046 |
| CD28 | 0.006111866 | 0.002885183 |
| PSMB5 | 1.853977181 | 0.002895208 |
| SERPINA1 | 36.87116527 | 0.002919575 |
| VCAN | 291.4721284 | 0.002972129 |
| CRABP2 | 13.70913155 | 0.002982129 |
| MX1 | 0.22237984 | 0.003006283 |
| MAGEA12 | 0.113617543 | 0.003053604 |
| RBL2 | 0.680005929 | 0.003083301 |
| EPCAM | 3.798899867 | 0.003099542 |
| IRF3 | 0.541308608 | 0.003123857 |
| FLNB | 5.379051478 | 0.003126239 |
| NT5E | 43.44305997 | 0.003131437 |
| TNFRSF11B | 2.672218521 | 0.003133748 |
| CYBB | 0.0077649 | 0.003164977 |
| IL15 | 2.986027798 | 0.003182823 |
| NFKBIA | 2.642960867 | 0.003186941 |
| CCL22 | 2.32970543 | 0.003207808 |
| GOT1 | 2.195378804 | 0.003216554 |
| HDAC11 | 2.424512739 | 0.00326801 |
| F2RL1 | 13.79509489 | 0.003283024 |
| IL1R2 | 2.390187723 | 0.003315878 |
| HMGA1 | 0.681440994 | 0.003327641 |
| FZD9 | 5.355176933 | 0.003340188 |
| BBC3 | 0.570087132 | 0.00343464 |
| TRIM21 | 1.555680929 | 0.003465796 |
| LAMB3 | 22.40820219 | 0.00346661 |
| FAS | 3.213216101 | 0.003498523 |
| IL2RG | 0.018966515 | 0.003587696 |
| PGPEP1 | 0.461240481 | 0.003628126 |
| DUSP2 | 0.144506287 | 0.0036499 |
| AREG | 78.32815965 | 0.003764576 |
| TGFB2 | 48.78857641 | 0.00382372 |
| CASP8 | 0.548382131 | 0.004037612 |
| PIK3R5 | 0.10734562 | 0.004055528 |
| CDH2 | 0.046242624 | 0.004081665 |
| RELA | 0.660846808 | 0.004132926 |
| RAD51 | 0.61582525 | 0.004157245 |
| CD48 | 0.225665764 | 0.004170836 |
| ICAM5 | 0.23319944 | 0.004217107 |
| HLA-E | 0.467887792 | 0.004217921 |
| AXIN1 | 0.54395761 | 0.004336441 |
| TNFAIP3 | 1.992625748 | 0.004375852 |
| CD45RA | 0.225097401 | 0.00441465 |
| GBP4 | 0.1739503 | 0.004457911 |
| COL6A3 | 5.197340798 | 0.004514992 |
| CD6 | 0.268967961 | 0.004523251 |
| CCL4 | 1.686251067 | 0.004559522 |
| CD40 | 1.977650074 | 0.00459049 |
| HK1 | 47.84902256 | 0.004623261 |
| OAS1 | 0.689506192 | 0.004651081 |
| CSF1 | 3.751487898 | 0.004664564 |
| CD58 | 2.293417167 | 0.00470492 |
| CSF2RB | 0.044018744 | 0.004813483 |
| COL5A1 | 29.86342893 | 0.004847782 |
| EXO1 | 0.681215238 | 0.004887728 |
| FYN | 21.27680412 | 0.004897729 |
| GBP1 | 2.717610698 | 0.004934261 |
| FGF13 | 0.193551958 | 0.005002491 |
| SREBF1 | 0.140066552 | 0.005057864 |
| TP53 | 2.688322629 | 0.00529716 |
| CLEC7A | 0.03308045 | 0.005305533 |
| NECTIN2 | 15.77635515 | 0.005441242 |
| PTGS2 | 5.944113264 | 0.00564166 |
| GIMAP4 | 0.033576861 | 0.005702319 |
| GIMAP6 | 0.113688291 | 0.005755358 |
| IL10RA | 0.019809157 | 0.005769222 |
| IFNAR1 | 0.565635485 | 0.005835915 |
| SHC2 | 3.714961307 | 0.005857804 |
| EPM2AIP1 | 0.792842019 | 0.005858124 |
| CCNE1 | 1.523407215 | 0.005875296 |
| ANGPT1 | 0.218496485 | 0.005958285 |
| BCAT1 | 2.70613382 | 0.005964759 |
| IL6 | 21.96851116 | 0.006013235 |
| IL11 | 80.81036184 | 0.006020708 |
| SLC1A5 | 0.42668854 | 0.006040235 |
| HLA-DPA1 | 0.395601572 | 0.006108888 |
| PDCD1LG2 | 5.482665179 | 0.00612732 |
| FOSL1 | 20.19527759 | 0.006375643 |
| HEY1 | 1.939391892 | 0.006393011 |
| CCL13 | 2.855587808 | 0.006549496 |
| LY96 | 0.257861706 | 0.006630203 |
| BCL6B | 3.613888889 | 0.006662444 |
| PPARGC1B | 0.642560721 | 0.006787691 |
| ICAM2 | 0.155497311 | 0.006902508 |
| IL7R | 57.09219858 | 0.006917811 |
| LOXL2 | 9.764854861 | 0.006970303 |
| STAT2 | 0.615572059 | 0.007039329 |
| CD300A | 0.028182715 | 0.007100933 |
| PIK3CG | 0.357477163 | 0.007104007 |
| WDR76 | 0.558004584 | 0.007277736 |
| PIK3R2 | 1.437198203 | 0.007409142 |
| MSH2 | 0.720066606 | 0.0074189 |
| TLR9 | 0.094117789 | 0.007493702 |
| MFGE8 | 2.054605704 | 0.007525327 |
| ITGA2 | 236.8242173 | 0.007679349 |
| MMP1 | 277.9806763 | 0.007688615 |
| C5 | 2.337118524 | 0.007763843 |
| IL24 | 5.740553343 | 0.007855873 |
| CASP9 | 0.375271749 | 0.007986902 |
| SPRY4 | 2.044457123 | 0.008077467 |
| TNFAIP6 | 3.772773797 | 0.008098046 |
| LAIR1 | 0.014036959 | 0.008294726 |
| CTAG1B | 0.034347656 | 0.00833511 |
| CDK2 | 1.262899303 | 0.008347272 |
| MYD88 | 1.532416224 | 0.008437621 |
| CENPF | 1.283179171 | 0.008463208 |
| KDR | 8.705941591 | 0.008577072 |
| EGF | 3.456806673 | 0.008602341 |
| S100A8 | 2.159268045 | 0.00861335 |
| SGK1 | 1.628722553 | 0.008762336 |
| MKI67 | 1.891374 | 0.008843087 |
| BRCA1 | 0.63417497 | 0.008853674 |
| RSAD2 | 4.933443696 | 0.008910218 |
| EZH2 | 0.581050912 | 0.009024276 |
| HLA-DQA1 | 2.894332373 | 0.009029714 |
| TLR5 | 11.91592428 | 0.009071559 |
| HMGB1 | 1.427769757 | 0.009074412 |
| FCGR2A | 0.023739174 | 0.009125099 |
| HLA-DRA | 0.060349385 | 0.009162785 |
| DKK1 | 87.16226708 | 0.009198891 |
| CSF2 | 19.97972222 | 0.009212201 |
| TNFRSF11A | 11.62013793 | 0.009275792 |
| TNFRSF14 | 0.342813325 | 0.009427111 |
| CSF1R | 23.53265869 | 0.009447479 |
| TREM1 | 7.809308788 | 0.009487786 |
| IFNGR1 | 1.281081372 | 0.009553928 |
| SERPINB5 | 83.73115578 | 0.009653204 |
| SLC11A1 | 2.372466216 | 0.00989694 |
| PDGFRB | 2.606668774 | 0.009940672 |
| ID4 | 4.573389997 | 0.009980799 |
| CES3 | 0.702205047 | 0.010012641 |
| STC1 | 33.8434663 | 0.010017335 |
| SOX2 | 5.386776293 | 0.010076491 |
| HAVCR2 | 0.015060556 | 0.010626859 |
| PNOC | 0.398878155 | 0.010912194 |
| HDAC3 | 1.490770723 | 0.010914119 |
| GNLY | 2.537461437 | 0.011006831 |
| SIRPB2 | 6.657074341 | 0.01107933 |
| PSMB9 | 0.738817924 | 0.011218559 |
| CXCL2 | 96.81142177 | 0.011337254 |
| IRF9 | 2.184493869 | 0.011505652 |
| NCR1 | 1.777170613 | 0.011771097 |
| ACVR1C | 1.559322034 | 0.011786691 |
| IL6R | 0.295608842 | 0.011810721 |
| TNFSF9 | 1.945848375 | 0.011928348 |
| PARP12 | 2.083704551 | 0.011932229 |
| PLOD2 | 402.6003335 | 0.012017082 |
| BRIP1 | 1.696396382 | 0.012075582 |
| CXCL16 | 0.282945808 | 0.012149071 |
| CD7 | 1.719874804 | 0.012152343 |
| MAP3K12 | 3.667514304 | 0.012212299 |
| MICA | 1.635790363 | 0.012334039 |
| CCL5 | 0.12548308 | 0.012353425 |
| ELOB | 0.552488419 | 0.01236522 |
| TNFSF10 | 0.484528455 | 0.012390736 |
| IL21R | 0.325859554 | 0.012452747 |
| OAS3 | 4.346288972 | 0.012798796 |
| BRD4 | 0.698402778 | 0.012898896 |
| CCNO | 1.807262164 | 0.013031742 |
| ATF3 | 0.516136687 | 0.013283039 |
| BATF3 | 1.787886785 | 0.013531077 |
| SPP1 | 0.000611337 | 0.013704988 |
| TNFRSF8 | 0.209085554 | 0.014293248 |
| NLRC5 | 0.452049136 | 0.014501677 |
| PDK1 | 0.53593764 | 0.014549525 |
| HERC6 | 3.622470795 | 0.014661667 |
| NRAS | 1.203519611 | 0.014720872 |
| COL17A1 | 151.4425149 | 0.01473486 |
| NEIL1 | 2.721073225 | 0.014782229 |
| WNT5B | 0.24346748 | 0.014929053 |
| SIRPA | 3.941717474 | 0.015004929 |
| BAX | 1.621227549 | 0.015279884 |
| CD79B | 0.482097547 | 0.015360963 |
| CXCL1 | 36.21369354 | 0.015369144 |
| AXL | 18.10600707 | 0.015507606 |
| EDN1 | 17.4861784 | 0.015653566 |
| IL1B | 2.003194888 | 0.015915186 |
| APH1B | 14.74598237 | 0.015978874 |
| NBN | 1.912178256 | 0.016087285 |
| PTEN | 0.730778467 | 0.016779964 |
| CXCL10 | 0.028791896 | 0.016798309 |
| NECTIN1 | 2.400324233 | 0.01699385 |
| DEPTOR | 0.127078979 | 0.017064954 |
| CXCL8 | 6.510123492 | 0.017126849 |
| FCGR2B | 0.354529677 | 0.017274978 |
| SNAI1 | 5.763219375 | 0.017333812 |
| IRF8 | 0.062393802 | 0.017499765 |
| MAP3K8 | 0.791354663 | 0.01772245 |
| EIF2B4 | 0.730870019 | 0.017819077 |
| IL18R1 | 2.474964006 | 0.017937713 |
| LILRB4 | 0.087162147 | 0.018092107 |
| CXCL9 | 0.056857851 | 0.01848327 |
| CCNA1 | 19.25232854 | 0.018621194 |
| BAMBI | 0.535237424 | 0.018871522 |
| LY9 | 0.091221419 | 0.019211082 |
| TWF1 | 5.929752858 | 0.01925003 |
| IFI35 | 1.08354262 | 0.01928318 |
| PRLR | 2.341899805 | 0.019303241 |
| GOT2 | 0.793663403 | 0.019478664 |
| LAMC2 | 295.4966284 | 0.019584253 |
| CHUK | 1.5944388 | 0.019702468 |
| GBP2 | 1.232134219 | 0.019770355 |
| MRC1 | 0.605515128 | 0.020311411 |
| ANGPTL4 | 68.9831815 | 0.020447885 |
| FPR1 | 4.436864153 | 0.020490443 |
| MAGEA3/A6 | 0.232850285 | 0.020590511 |
| CEBPB | 2.279111105 | 0.020678524 |
| TAPBPL | 0.633239653 | 0.020898191 |
| HCK | 0.495788207 | 0.020907322 |
| HDAC4 | 0.743146008 | 0.021195476 |
| FGF18 | 2.300036996 | 0.021519667 |
| PDZK1IP1 | 0.288467768 | 0.021614024 |
| IFI27 | 4.423376462 | 0.022606437 |
| FAP | 3.912942173 | 0.023200956 |
| VEGFA | 2.214182332 | 0.023484474 |
| S100A9 | 3.601515258 | 0.023498689 |
| IRF1 | 0.703569544 | 0.023602497 |
| P2RY13 | 1.581804281 | 0.024356065 |
| TGFB1 | 0.73266677 | 0.024594137 |
| AQP9 | 2.335757977 | 0.02485619 |
| IFNA1 | 1.97950014 | 0.025530242 |
| COMP | 2.03746594 | 0.026095186 |
| NGFR | 1.671117812 | 0.026472396 |
| PTGER4 | 2.223390948 | 0.026585255 |
| TICAM1 | 1.35506581 | 0.026707891 |
| SAMSN1 | 0.050030952 | 0.027620847 |
| FOXP3 | 2.02022129 | 0.028564804 |
| DUSP5 | 0.613460732 | 0.02857385 |
| RPS6KB1 | 0.858258759 | 0.02872949 |
| MS4A4A | 0.140925836 | 0.028904429 |
| CSF3 | 1.527572016 | 0.028986222 |
| CD45RO | 0.283591065 | 0.029045097 |
| HLA-DRB1 | 0.145702063 | 0.029249498 |
| TNF | 2.239139663 | 0.029919806 |
| SAMD9 | 1.34119756 | 0.030165115 |
| REN | 2.113347458 | 0.030402661 |
| FCGRT | 1.651759905 | 0.030692243 |
| PDGFA | 1.704716998 | 0.031068666 |
| DEFB134 | 2.462809917 | 0.031349959 |
| CMKLR1 | 0.293040703 | 0.031378626 |
| FZD8 | 1.348660126 | 0.031441116 |
| CEP55 | 1.903498916 | 0.032106744 |
| CCL3/L1 | 0.273473261 | 0.032413688 |
| ITGAL | 0.287950664 | 0.032570927 |
| PARP4 | 0.755118668 | 0.032578709 |
| ZAP70 | 0.160269122 | 0.03270298 |
| NCAM1 | 0.031808231 | 0.033367299 |
| PIAS4 | 1.266541927 | 0.033472837 |
| C2 | 0.333253051 | 0.034268624 |
| IL2 | 1.672740138 | 0.034324343 |
| CD14 | 2.482232346 | 0.034582459 |
| GZMM | 1.676544091 | 0.034792819 |
| TCF3 | 0.885405963 | 0.035153018 |
| MAGEC2 | 0.093479815 | 0.035409284 |
| ADM | 0.543011365 | 0.035471672 |
| WNT5A | 0.362614913 | 0.036659804 |
| IGF2R | 0.832539802 | 0.036664067 |
| IFNGR2 | 0.749816196 | 0.037593258 |
| IL32 | 5.052968533 | 0.037902717 |
| TNFRSF4 | 2.201582106 | 0.038190092 |
| PARP9 | 0.849243479 | 0.038798085 |
| PROM1 | 1.344952318 | 0.039170512 |
| THY1 | 1.882909255 | 0.039257443 |
| IFIT1 | 2.144477899 | 0.039729915 |
| ERO1A | 3.803819428 | 0.039779076 |
| ITGAX | 1.349596088 | 0.039895756 |
| HLA-DOB | 0.439437619 | 0.040775971 |
| APOE | 10.45788901 | 0.040867735 |
| SBNO2 | 1.380908323 | 0.042351929 |
| MSH6 | 0.735432593 | 0.042627539 |
| NID2 | 1.209015294 | 0.042746996 |
| HLA-DRB5 | 3.167113494 | 0.044169633 |
| IL11RA | 1.824319177 | 0.044760712 |
| CD8A | 0.624841471 | 0.046035715 |
| FBP1 | 3.113765616 | 0.04673887 |
| CDKN1A | 4.540434393 | 0.046751223 |
| ICAM1 | 0.785926464 | 0.046860368 |
| SPIB | 0.561259466 | 0.046990785 |
| SFXN1 | 1.107478391 | 0.047050208 |
| TNFSF4 | 0.295261155 | 0.047447135 |
| BIRC5 | 0.759203089 | 0.048084233 |
| MS4A1 | 0.28258882 | 0.048593591 |
| KIT | 0.08085626 | 0.049129911 |
| MRE11 | 0.87848235 | 0.049377248 |
| IDO1 | 3.451156812 | 0.050492631 |

TABLE 13
Genes Differentially Expressed in FaDu
tumors vs Raji tumors at baseline

| | FaDu control | |
|---|---|---|
| | IgG/Raji | |
| Gene Name | control IgG | p-value |
| GPSM3 | 0.030335281 | 6.99429E−08 |
| IL10 | 0.029429498 | 2.78173E−07 |
| ARID1A | 0.42613065 | 1.84082E−06 |
| BLK | 0.043565114 | 2.56233E−06 |
| FADD | 5.55768871 | 2.82155E−06 |
| F2RL1 | 46.80558598 | 3.9077E−06 |
| ENO1 | 0.470447691 | 6.24596E−06 |
| RNLS | 5.308201831 | 6.4497E−06 |
| ADORA2A | 0.092472987 | 6.57272E−06 |
| RELA | 2.651382271 | 8.03387E−06 |
| ITPK1 | 3.815626632 | 9.44695E−06 |
| SYK | 0.141772611 | 1.09205E−05 |
| APH1B | 3.466483605 | 1.12835E−05 |
| ENTPD1 | 0.08137522 | 1.51462E−05 |
| SLAMF7 | 0.041423593 | 2.36831E−05 |
| MET | 5.176179889 | 2.5827E−05 |
| CD79A | 0.002884037 | 3.05727E−05 |
| MSH6 | 0.384740494 | 4.64377E−05 |
| SOCS1 | 0.066841099 | 4.68644E−05 |
| TYMS | 0.474166592 | 4.88655E−05 |
| ATM | 0.333941788 | 5.18244E−05 |
| RB1 | 0.475772494 | 5.51011E−05 |
| BAX | 0.433928703 | 5.58607E−05 |
| PTCD2 | 0.536961892 | 5.7398E−05 |
| CTSS | 0.13325717 | 6.76123E−05 |
| HLA-DQA1 | 0.000631934 | 6.83223E−05 |
| NCR1 | 0.32723068 | 6.88609E−05 |
| ICAM1 | 0.188190043 | 7.0416E−05 |
| MAGEA1 | 6.952792598 | 7.41931E−05 |
| EIF2B4 | 0.411518132 | 7.7145E−05 |
| FANCA | 0.254149662 | 8.58215E−05 |
| PTGER4 | 0.225211036 | 9.37601E−05 |
| RRM2 | 0.379277532 | 9.41521E−05 |
| CD247 | 0.18494745 | 9.79142E−05 |
| CASP9 | 0.34084018 | 0.000115102 |
| GLUD1 | 0.412777513 | 0.000122554 |
| HLA-DQA2 | 0.149429687 | 0.000129157 |
| NF1 | 2.740266957 | 0.000129644 |
| RELB | 0.221449213 | 0.000131792 |
| TGFB1 | 0.235627466 | 0.000140015 |
| IFITM2 | 4.705766303 | 0.000142037 |
| TNFSF8 | 0.245429794 | 0.000147507 |
| LRRC32 | 0.256254856 | 0.00015452 |
| MKI67 | 0.727473166 | 0.000172975 |
| SLC7A5 | 3.046616045 | 0.000174147 |
| NFKB1 | 0.717154281 | 0.000186377 |
| DUSP2 | 0.053425942 | 0.00018698 |
| CX3CL1 | 6.654963294 | 0.000220398 |
| PIK3R1 | 0.217806796 | 0.000222167 |
| ANLN | 2.588117513 | 0.000245078 |
| CCL14 | 0.338718327 | 0.000254569 |
| TLR9 | 0.022396438 | 0.000262054 |
| E2F3 | 0.541576782 | 0.000263465 |
| CD14 | 2.728361459 | 0.000263475 |
| IL21R | 0.057429563 | 0.000272988 |
| PPARG | 2.956950919 | 0.000277668 |
| CD74 | 0.002094132 | 0.000279014 |
| PSMB9 | 0.356016881 | 0.000280165 |
| SLC1A5 | 0.445871699 | 0.000281373 |
| ALDOA | 1.748584247 | 0.000292119 |
| HLA-DPB1 | 0.002584834 | 0.000293696 |
| HEY1 | 0.098031524 | 0.000297069 |
| NFKBIA | 1.398814259 | 0.000299721 |
| ERO1A | 1.874391824 | 0.000303431 |
| ITGB2 | 0.064902748 | 0.000304043 |
| IFI16 | 0.484452705 | 0.000310899 |
| IL10RA | 0.022648731 | 0.000348223 |
| SFXN1 | 0.632610502 | 0.000348852 |
| MS4A1 | 0.000998068 | 0.000352262 |
| FCGR2B | 0.008232199 | 0.000379211 |
| CD28 | 0.462595702 | 0.000379752 |
| CD79B | 0.00378755 | 0.000392107 |
| CD3G | 0.266496562 | 0.000403133 |
| HLA-DPA1 | 0.001721844 | 0.000419198 |
| MAGEC1 | 0.314914343 | 0.000436974 |
| ITGAX | 0.573430762 | 0.000467856 |
| KLRD1 | 0.277427491 | 0.000490881 |
| HLA-DRA | 0.003143794 | 0.00050256 |
| WNT5A | 0.041592297 | 0.000517444 |
| MB21D1 | 18.96241933 | 0.000525078 |
| CD70 | 0.032578382 | 0.000540849 |
| PTPRC | 0.003969337 | 0.000543612 |
| LY9 | 0.018192651 | 0.000567351 |
| GPC4 | 4.514180374 | 0.000573387 |
| FYN | 0.203940272 | 0.000574108 |
| MAP3K8 | 0.111039543 | 0.000637886 |
| PNOC | 0.135169548 | 0.000639124 |
| IL18 | 85.08930426 | 0.000640762 |
| MSH2 | 0.363942119 | 0.000678377 |
| HRAS | 1.719494737 | 0.000701515 |
| PSMB10 | 0.390988567 | 0.00070205 |
| PIAS4 | 0.657379237 | 0.000720142 |
| MAP3K5 | 0.126678264 | 0.000724517 |
| ITGA4 | 0.002401846 | 0.000728022 |
| CD80 | 0.010344343 | 0.000734182 |
| CXorf36 | 0.350974432 | 0.000753443 |
| CBLC | 2.840199185 | 0.000776115 |
| BCAT1 | 0.208326396 | 0.000784966 |
| PMS2 | 0.573793828 | 0.000801369 |
| BMP2 | 5.642216789 | 0.000804252 |
| IDO1 | 0.297413793 | 0.000807055 |
| HLA-DMB | 0.011414176 | 0.00080722 |
| VEGFB | 0.360645815 | 0.000808197 |
| SLC2A1 | 1.728570752 | 0.000818785 |
| SERPINH1 | 20.37642379 | 0.000843162 |
| BCL2 | 0.015890689 | 0.00086396 |
| SFRP1 | 0.346685493 | 0.000877539 |
| MICB | 0.701754261 | 0.000883344 |
| DEPTOR | 0.331519805 | 0.000885972 |
| MTOR | 0.460976318 | 0.000920709 |
| FGF18 | 0.261956646 | 0.00092864 |
| CCNE1 | 0.60769165 | 0.00093327 |
| FAM30A | 0.037024627 | 0.000934057 |
| CCND1 | 185.6335093 | 0.000948945 |
| THBS1 | 75.45720785 | 0.000960228 |
| COL11A1 | 0.445411194 | 0.000965889 |
| TNFRSF17 | 0.033572571 | 0.000971574 |
| RPS6KB1 | 0.613843082 | 0.000974869 |
| EPM2AIP1 | 0.465128251 | 0.000983392 |
| BRCA2 | 0.71145473 | 0.000996102 |
| CD38 | 0.006719205 | 0.001011495 |
| TPM1 | 15.32043682 | 0.001011635 |
| LY96 | 0.034968789 | 0.001043248 |
| SLC16A1 | 1.201299252 | 0.001048423 |
| PPARGC1B | 0.192889382 | 0.001051662 |
| NFATC2 | 0.096288183 | 0.001078843 |
| IFNAR1 | 2.163188833 | 0.001092807 |
| DNMT1 | 0.316292435 | 0.001093686 |
| NFKB2 | 0.293164851 | 0.00111557 |
| EDN1 | 2.595739834 | 0.001136421 |
| CD27 | 0.002848484 | 0.001139298 |
| EIF5AL1 | 0.323268016 | 0.001157414 |
| PLA1A | 0.220464185 | 0.001185762 |
| HLA-DOB | 0.026903819 | 0.001192684 |
| HLA-DOA | 0.047651197 | 0.001197196 |
| ULBP2 | 2.325573077 | 0.001208612 |
| FGFR1 | 0.286059577 | 0.001208987 |
| CD48 | 0.001207337 | 0.00121003 |
| NECT1N1 | 6.188555289 | 0.00121912 |
| CDKN2A | 0.598776572 | 0.001224368 |
| EZH2 | 0.41760561 | 0.001262484 |
| TNFRSF11B | 0.388095238 | 0.001266485 |
| CCNA1 | 20.09466153 | 0.001282842 |
| CDK6 | 0.49260888 | 0.001290935 |
| CD276 | 40.76098935 | 0.001320208 |
| TNFSF13 | 2.656675749 | 0.001322505 |
| NFKBIE | 0.134579015 | 0.001370042 |
| RAD51 | 0.610051623 | 0.001395982 |
| MYD88 | 0.408425043 | 0.001409019 |
| MAP3K7 | 1.314926269 | 0.001418124 |
| BIRC3 | 0.101177394 | 0.001423204 |
| EIF4EBP1 | 4.431154013 | 0.001455034 |
| TNF | 0.047008742 | 0.001479499 |
| TMEM173 | 9.270549599 | 0.001516223 |
| HLA-DQB1 | 0.023419783 | 0.001523729 |
| HIF1A | 0.616474682 | 0.001537134 |
| IL4 | 0.260755048 | 0.001567419 |
| CEACAM3 | 0.253868472 | 0.001576502 |
| NDUFA4L2 | 0.536200887 | 0.001608863 |
| CDC20 | 1.99288892 | 0.001622811 |
| WDR76 | 0.509764379 | 0.001635515 |
| RUNX3 | 0.028547308 | 0.001642252 |
| CD3E | 0.280319629 | 0.001693127 |
| ITGAE | 0.49956533 | 0.001705967 |
| CYBB | 0.005686522 | 0.001717794 |
| TNFRSF1A | 19.1902153 | 0.00172418 |
| NECTIN2 | 21.94654556 | 0.001725114 |
| MAGEA4 | 20.59037711 | 0.001754472 |
| IL2RA | 0.330104267 | 0.00175875 |
| S100A9 | 388.379179 | 0.001766215 |
| IRF8 | 0.003107448 | 0.001782942 |
| PFKM | 0.394466176 | 0.00179193 |
| TBXAS1 | 0.282574874 | 0.00181616 |
| ADM | 22.19034031 | 0.001831602 |
| BRD3 | 3.105408921 | 0.001861295 |
| ARNT2 | 3.151543675 | 0.001866391 |
| VCAM1 | 0.232798957 | 0.001870078 |
| EPCAM | 29.81225022 | 0.001893192 |
| C5 | 0.285312085 | 0.001929603 |
| STAT1 | 2.39061785 | 0.001940184 |
| IL16 | 0.008488627 | 0.001948845 |
| LILRA1 | 0.266904872 | 0.001963422 |
| FCGR2A | 0.118365788 | 0.001965263 |
| LYZ | 0.246164274 | 0.001977783 |
| BRD4 | 0.660496015 | 0.001978377 |
| COL17A1 | 57.5125872 | 0.001986939 |
| TNFSF9 | 0.155103649 | 0.002046842 |
| HK1 | 1.446420835 | 0.002054389 |
| PIK3CD | 0.024982912 | 0.00207812 |
| MS4A4A | 0.245275367 | 0.002111952 |
| CCL2 | 0.197036313 | 0.002117965 |
| ITGA6 | 3.320803303 | 0.00212787 |
| PECAM1 | 0.248658529 | 0.002158819 |
| CCNB1 | 2.224421009 | 0.002165766 |
| HLA-DRB1 | 0.002276877 | 0.002232428 |
| GZMK | 0.441017812 | 0.002315782 |
| TNFRSF4 | 0.1216296 | 0.00235611 |
| EIF2AK2 | 1.792327562 | 0.002396512 |
| PVR | 4.389540278 | 0.002482396 |
| BIRC5 | 1.780229251 | 0.002490263 |
| KAT2B | 0.266378154 | 0.002535626 |
| RIPK2 | 0.288719444 | 0.002565348 |
| HLA-DMA | 0.008284587 | 0.002611287 |
| CSF2RB | 0.070360063 | 0.00265686 |
| GZMM | 0.262168142 | 0.002709095 |
| TCF3 | 0.167534398 | 0.002727684 |
| KLRB1 | 0.353127079 | 0.002743683 |
| CCR2 | 0.367468322 | 0.002759937 |
| SERPINB5 | 25.78718798 | 0.002781699 |
| CD84 | 0.002656745 | 0.002827958 |
| BBS1 | 2.502700164 | 0.002857109 |
| ZEB2 | 0.028674879 | 0.002911384 |
| S100A8 | 53.81817049 | 0.002916084 |
| DKK1 | 56.20523752 | 0.002944176 |
| CHUK | 0.570377165 | 0.003015249 |
| SGK1 | 0.462626226 | 0.003031328 |
| NRAS | 1.260487861 | 0.003041795 |
| IL2RG | 0.003599101 | 0.003048871 |
| PLOD2 | 90.78794403 | 0.003092373 |
| TAF3 | 0.489354024 | 0.003093824 |
| HELLS | 0.535370341 | 0.003170024 |
| IL7R | 2.027567531 | 0.00320269 |
| TLR1 | 0.027793516 | 0.003235689 |
| CPA3 | 0.32116122 | 0.003242447 |
| PGPEP1 | 0.21613584 | 0.003285887 |
| HLA-A | 0.366476716 | 0.003291759 |
| PC | 11.68429738 | 0.003395174 |
| TNFSF4 | 0.17136294 | 0.003424297 |
| HLA-F | 0.469317062 | 0.003426359 |
| CLECL1 | 0.001657854 | 0.003428752 |
| LTBP1 | 0.076592056 | 0.003448824 |
| CD40 | 0.0323077 | 0.003449449 |
| TNFRSF14 | 0.082747557 | 0.003490732 |
| CXCR4 | 0.011618508 | 0.003535569 |
| VCAN | 1.955298845 | 0.003578674 |
| CEBPB | 7.406710665 | 0.003646886 |
| JAG1 | 50.90509612 | 0.003850317 |
| GMIP | 0.185512208 | 0.003905951 |
| NID2 | 0.580920688 | 0.00391334 |
| CD44 | 42.97467992 | 0.003944292 |
| PRLR | 2.490777052 | 0.003948847 |
| ICOSLG | 0.15435978 | 0.003949817 |
| TAP2 | 0.518729654 | 0.003961408 |
| OAS3 | 112.4719783 | 0.003998794 |
| SPIB | 0.01533097 | 0.004008109 |
| NLRC5 | 0.482756742 | 0.004042696 |
| DLL1 | 44.51055495 | 0.0041026 |
| CCL4 | 0.009661495 | 0.004170695 |
| SAMD9 | 14.73610952 | 0.004180688 |
| RELN | 0.238266125 | 0.004188177 |
| GLI1 | 0.369105885 | 0.004230017 |
| LAMB3 | 144.7987988 | 0.004283172 |
| CCND2 | 11.80801078 | 0.004315782 |
| FCN1 | 0.423602033 | 0.004328273 |
| ICAM2 | 0.015705354 | 0.004332782 |
| TCL1A | 0.002409878 | 0.004366941 |
| CDKN1C | 3.394744929 | 0.004367271 |
| NOTCH2 | 2.443473179 | 0.004388065 |
| WNT7B | 14.02818057 | 0.004428693 |
| MAPK10 | 0.403631538 | 0.004439579 |
| CD86 | 0.014100963 | 0.00449488 |
| IFITM1 | 8.649540423 | 0.004506545 |
| FPR1 | 0.268491228 | 0.004578402 |
| GNG4 | 0.465678627 | 0.004591724 |
| IL2RB | 0.272942211 | 0.004641433 |
| MFNG | 0.066276442 | 0.004852007 |
| IL11RA | 0.444243494 | 0.004858984 |
| AXIN1 | 0.742375632 | 0.004872138 |
| IL1B | 19.83040437 | 0.004902044 |
| ICAM5 | 0.373687688 | 0.004927124 |
| MLH1 | 0.574742992 | 0.004935475 |
| JAG2 | 5.373947972 | 0.004949936 |
| ERBB2 | 16.73667453 | 0.004962354 |
| RPL23 | 0.414413755 | 0.00496489 |
| MAML2 | 0.198979729 | 0.004971628 |
| RPTOR | 1.453171323 | 0.005058646 |
| BID | 0.513398485 | 0.005090067 |
| P4HA2 | 3.544015499 | 0.005107858 |
| TNFRSF10B | 1.937048189 | 0.005292425 |
| PIK3CG | 0.065775335 | 0.005325808 |
| VEGFA | 4.588925678 | 0.005326592 |
| MYC | 0.658233803 | 0.005353587 |
| EGFR | 57.19575843 | 0.005355464 |
| BNIP3 | 2.214662023 | 0.005399466 |
| SERPINA1 | 3.887239264 | 0.005428935 |
| TRAF1 | 0.025465072 | 0.005448512 |
| CCR5 | 0.243658724 | 0.005508489 |
| IHH | 0.246757641 | 0.005517828 |
| CSF3R | 0.392234432 | 0.005556662 |
| IRF4 | 0.035532965 | 0.005566254 |
| CD45RA | 0.001827593 | 0.005598231 |
| OLFML2B | 0.185444744 | 0.005657111 |
| PF4 | 0.320618408 | 0.005702187 |
| CTLA4 | 0.450178238 | 0.00573903 |
| FLNB | 1.461553316 | 0.005765268 |
| OASL | 40.25589818 | 0.005806229 |
| FCRL2 | 0.017310894 | 0.005922397 |
| ARG2 | 0.340834552 | 0.005934886 |
| ITGAV | 6.071898566 | 0.006024869 |
| ITGAL | 0.047304874 | 0.006056926 |
| CSF1R | 0.146008849 | 0.006081062 |
| LDHA | 0.59146083 | 0.006120337 |
| SPP1 | 0.237511085 | 0.006149468 |
| RIPK1 | 1.972632951 | 0.006199942 |
| CTNNB1 | 1.807447105 | 0.006290478 |
| IL6 | 7.61222134 | 0.006306088 |
| ZEB1 | 0.032211969 | 0.00638142 |
| POLD1 | 0.589163532 | 0.006461274 |
| HDAC11 | 1.81632494 | 0.006481573 |
| CD19 | 0.0045805 | 0.006641599 |
| ITGB3 | 0.417768648 | 0.006650148 |
| KDR | 0.275412294 | 0.006724494 |
| LCK | 0.080432529 | 0.00675123 |
| TWF1 | 1.726953707 | 0.006794262 |
| FCGR3A/B | 0.423539132 | 0.006804646 |
| BRCA1 | 0.64356525 | 0.006979721 |
| IRF5 | 0.586059357 | 0.007004864 |
| AXL | 12.21636862 | 0.007005997 |
| ITGB8 | 38.89761547 | 0.007057789 |
| CCL22 | 0.028695921 | 0.007186074 |
| PDGFA | 4.353797591 | 0.007203485 |
| IRF3 | 0.406153446 | 0.007310753 |
| CCL19 | 0.416666667 | 0.007395338 |
| CLEC5A | 0.250431253 | 0.007441399 |
| CD69 | 0.012791308 | 0.00758589 |
| IRF1 | 0.584396077 | 0.007593382 |
| PSMB8 | 0.478799852 | 0.007602931 |
| NBN | 0.511289459 | 0.007684357 |
| TICAM1 | 0.331124507 | 0.007701335 |
| IFIT1 | 6.788157087 | 0.007874714 |
| TNFRSF10D | 0.38513194 | 0.007904788 |
| CD47 | 0.726743745 | 0.0079058 |
| CDH1 | 58.8345689 | 0.007937657 |
| GHR | 0.655775528 | 0.008019401 |
| SAMSN1 | 0.029895832 | 0.008035739 |
| HMGA1 | 0.685454194 | 0.008094816 |
| IL17A | 0.285041997 | 0.008155016 |
| LILRB2 | 0.146776144 | 0.008349801 |
| FSTL3 | 3.731888545 | 0.008365802 |
| LILRB4 | 0.297525309 | 0.008397075 |
| EOMES | 5.715083799 | 0.008412625 |
| IFI27 | 52.14786634 | 0.008581814 |
| CD96 | 0.177356201 | 0.008643118 |
| LILRA3 | 0.186171032 | 0.008699538 |
| CXCR3 | 0.265580448 | 0.008749746 |
| GPR160 | 0.350504799 | 0.008891261 |
| PRR5 | 2.033173105 | 0.009030778 |
| NOTCH1 | 9.302261669 | 0.00911464 |
| MMP1 | 50.9706532 | 0.009136873 |
| CCND3 | 0.278655479 | 0.00915129 |
| CXCL12 | 0.314536478 | 0.009354612 |
| IFIT3 | 3.131009736 | 0.009358409 |
| IRF9 | 2.619659443 | 0.009467263 |
| ITGA2 | 12.38923999 | 0.009503202 |
| GIMAP6 | 0.323656378 | 0.009563708 |
| FCAR | 0.348081683 | 0.009564424 |
| ANGPT2 | 0.235386769 | 0.009567004 |
| RIPK3 | 0.427329193 | 0.009631423 |
| WNT10A | 0.352921976 | 0.009686811 |
| NGFR | 2.026278907 | 0.009702707 |
| CASP3 | 0.661088518 | 0.009843953 |
| MICA | 2.521082042 | 0.009856404 |
| CD40LG | 0.194029851 | 0.009959295 |
| LTB | 0.017137796 | 0.010008839 |
| CRABP2 | 5.668143679 | 0.010176228 |
| CCL21 | 0.419870038 | 0.010332657 |
| PVRIG | 0.124628388 | 0.010510314 |
| VEGFC | 16.8066622 | 0.010533406 |
| COL5A1 | 10.75209003 | 0.010593493 |
| CD68 | 2.439532006 | 0.010681004 |
| IFI6 | 3.294868596 | 0.01073457 |
| FCGRT | 2.522160948 | 0.010785892 |
| SLC11A1 | 0.228346457 | 0.010812941 |
| BNIP3L | 1.376899082 | 0.010822086 |
| UBE2T | 0.462490957 | 0.010832361 |
| APLNR | 0.320725589 | 0.011034626 |
| IRF2 | 0.413734013 | 0.011043201 |
| SMAD5 | 0.665275466 | 0.011102944 |
| CCL3/L1 | 0.001778894 | 0.011164808 |
| SBNO2 | 1.597010372 | 0.011286791 |
| STAT3 | 1.758320581 | 0.011294065 |
| SRP54 | 0.728583169 | 0.011606077 |
| TP53 | 0.332622681 | 0.011657193 |
| PRKACB | 0.677268685 | 0.011705656 |
| CD2 | 0.436776636 | 0.011741152 |
| CXCL1 | 31.84106227 | 0.011775715 |
| CXCL13 | 0.201626223 | 0.011821939 |
| STAT4 | 4.884866469 | 0.012065918 |
| MRE11 | 0.687545812 | 0.012078205 |
| TNFRSF10C | 0.345696835 | 0.012088629 |
| CCL5 | 4.428780989 | 0.01240215 |
| IL2 | 0.385975929 | 0.012435913 |
| PRF1 | 0.313196336 | 0.01251374 |
| RICTOR | 1.440415525 | 0.01253355 |
| BCL6B | 0.449175947 | 0.01259986 |
| NFAM1 | 0.507322383 | 0.012623522 |
| RAD50 | 0.763759901 | 0.012655798 |
| HMGB1 | 1.797421312 | 0.012660864 |
| PARP12 | 3.375845804 | 0.012665627 |
| IL22RA1 | 0.488461868 | 0.012673771 |
| IFIH1 | 0.598604029 | 0.012762958 |
| IL1A | 75.24752297 | 0.012816582 |
| TPSAB1/B2 | 0.329835265 | 0.013139129 |
| HK2 | 1.560049337 | 0.013231908 |
| MX1 | 152.4291142 | 0.013263049 |
| GLUL | 2.934650039 | 0.013479958 |
| S100A12 | 0.596985357 | 0.013846834 |
| ITGAM | 0.318732662 | 0.013879514 |
| HSD11B1 | 0.413314257 | 0.013932718 |
| FBP1 | 2.159987903 | 0.013946796 |
| IFIT2 | 12.0773356 | 0.013991956 |
| NEIL1 | 0.152829134 | 0.014179858 |
| HLA-C | 1.93904304 | 0.014210628 |
| KIF2C | 1.245553601 | 0.014218369 |
| FOSL1 | 179.1985531 | 0.014298206 |
| FAM124B | 0.47224626 | 0.014434425 |
| CX3CR1 | 0.179553265 | 0.01459675 |
| TLR7 | 0.134433599 | 0.014641443 |
| CXCL5 | 0.345929339 | 0.014672286 |
| TNFRSF25 | 3.250486855 | 0.014736476 |
| FZD8 | 1.564627035 | 0.014955344 |
| VHL | 0.529356511 | 0.014963223 |
| ISG15 | 2.053519894 | 0.015120918 |
| FCGR1A | 0.182936029 | 0.01512223 |
| HDAC3 | 0.845303172 | 0.015140344 |
| OTOA | 0.150191734 | 0.015278593 |
| BCL2L1 | 0.765894818 | 0.015584982 |
| TYMP | 5.505724575 | 0.015592292 |
| TMEM140 | 0.317461188 | 0.015897386 |
| PCK2 | 0.442269633 | 0.016015796 |
| HES1 | 2.91607322 | 0.016122461 |
| PRKX | 0.363274176 | 0.016613799 |
| HERC6 | 18.44474937 | 0.016833921 |
| VTCN1 | 0.723353795 | 0.017023592 |
| GBP1 | 2.469613772 | 0.017033611 |
| CXCL9 | 0.101863753 | 0.017301844 |
| CXCL2 | 65.75272162 | 0.017360441 |
| FAS | 0.507149201 | 0.017499592 |
| CD300A | 0.390374787 | 0.017618286 |
| TNFRSF8 | 0.333469555 | 0.017856793 |
| GLS | 0.451420019 | 0.017860448 |
| NKG7 | 0.050976963 | 0.017965608 |
| LAMC2 | 39.03241005 | 0.017991363 |
| PLA2G2A | 0.312535238 | 0.018002809 |
| TLR2 | 2.675363862 | 0.018009847 |
| CLEC14A | 0.522569444 | 0.018155623 |
| CES3 | 0.162588598 | 0.018229655 |
| MELK | 0.850789959 | 0.018415465 |
| SOX11 | 0.247072392 | 0.018600808 |
| CCR4 | 0.500449457 | 0.018761772 |
| AQP9 | 0.185929357 | 0.018932573 |
| CCL20 | 11.49819093 | 0.019007419 |
| CASP8 | 0.3189755 | 0.019021513 |
| SELE | 0.391451415 | 0.01941404 |
| CXCL3 | 4.872872668 | 0.019422173 |
| CCL7 | 0.335153335 | 0.019710285 |
| MGMT | 1.624546849 | 0.019838276 |
| NFIL3 | 2.080610212 | 0.019984784 |
| RSAD2 | 3.421078488 | 0.019989806 |
| PDCD1 | 0.302555311 | 0.020717596 |
| JAK3 | 0.256116881 | 0.020784999 |
| CD8B | 0.460332912 | 0.02113985 |
| TTGIT | 0.498553813 | 0.021228779 |
| ATF3 | 3.648117973 | 0.021271478 |
| SHC2 | 0.348148148 | 0.021274597 |
| ROR2 | 0.670381483 | 0.02129291 |
| PDGFRB | 0.31374192 | 0.02129577 |
| HAVCR2 | 0.157996305 | 0.021422148 |
| TDO2 | 0.405295975 | 0.021588879 |
| TLR3 | 12.52743782 | 0.021699925 |
| LILRA5 | 0.396348252 | 0.021885396 |
| SMAP1 | 0.557257611 | 0.022175751 |
| MS4A2 | 0.300640439 | 0.02221397 |
| PARP9 | 1.821046199 | 0.022304987 |
| PTEN | 0.756935762 | 0.022445873 |
| TNFRSF9 | 0.347499428 | 0.022464424 |
| TGFBR1 | 0.399485984 | 0.022606182 |
| IL1RN | 2.20559178 | 0.022855092 |
| B2M | 0.754969645 | 0.023158863 |
| CXCL16 | 4.422726828 | 0.023254682 |
| EGR1 | 5.585290966 | 0.023454785 |
| ALDOC | 0.707506871 | 0.023878832 |
| KIT | 0.311867936 | 0.024241919 |
| CD4 | 0.303914591 | 0.024312636 |
| OLR1 | 5.095165641 | 0.024441928 |
| JAK1 | 0.618613518 | 0.024564143 |
| COL4A5 | 11.19502165 | 0.024585157 |
| EXO1 | 0.713923362 | 0.024630136 |
| TNFRSF1B | 0.021644413 | 0.024742044 |
| IFNA1 | 0.327277861 | 0.024769542 |
| VSIR | 2.99064707 | 0.025120861 |
| IKBKB | 2.168283093 | 0.025442694 |
| CD7 | 0.26716626 | 0.025497125 |
| ICAM3 | 0.136986037 | 0.025544603 |
| AREG | 64.70424092 | 0.025614657 |
| LIF | 20.95578016 | 0.025748863 |
| CTAG1B | 0.323932092 | 0.025817843 |
| ICOS | 0.282813782 | 0.026165829 |
| P4HA1 | 0.679436789 | 0.026794931 |
| STC1 | 10.03827813 | 0.027041483 |
| MS4A6A | 0.212024757 | 0.027073315 |
| TNFSF12 | 2.716586151 | 0.027155944 |
| IFI35 | 1.65171532 | 0.027715794 |
| BTLA | 0.274135447 | 0.027739003 |
| CD45RO | 0.407275954 | 0.027935374 |
| ARG1 | 0.418796016 | 0.02807971 |
| CNTFR | 0.357337485 | 0.028596439 |
| CDC25C | 1.260200943 | 0.028801023 |
| CXCR2 | 0.525227505 | 0.029030702 |
| SOX2 | 2.431144985 | 0.029105712 |
| CXCL6 | 0.429843693 | 0.029497993 |
| GBP4 | 6.08513181 | 0.030007444 |
| C5AR1 | 0.259818731 | 0.030016018 |
| IL1R2 | 0.050802152 | 0.030351301 |
| TREM2 | 0.443524746 | 0.030870689 |
| BBC3 | 0.472530196 | 0.030971073 |
| CCL18 | 0.62929325 | 0.031177325 |
| IER3 | 11.37923851 | 0.031291836 |
| CDKN1A | 1.551184979 | 0.031354467 |
| COMP | 0.377956719 | 0.031367817 |
| TWIST2 | 0.344539167 | 0.031676588 |
| ELOB | 0.552884166 | 0.031988497 |
| HCK | 0.307486941 | 0.032074663 |
| THBD | 2.468757369 | 0.032083458 |
| CD244 | 0.136770854 | 0.032360787 |
| CXCL10 | 0.069538428 | 0.032663429 |
| GZMH | 0.417308564 | 0.032776545 |
| MAGEB2 | 0.514444903 | 0.032783836 |
| CD5 | 0.277006375 | 0.032864882 |
| C1QA | 0.133559881 | 0.032929232 |
| PDZK1IP1 | 6.237711655 | 0.032945182 |
| CDH5 | 0.404537775 | 0.033048215 |
| SIGLEC5 | 0.361414712 | 0.033306476 |
| SIGLEC1 | 0.381292709 | 0.033378976 |
| WNT2 | 0.46983866 | 0.033485926 |
| MMP7 | 1.581006979 | 0.033624688 |
| CDK2 | 1.131979054 | 0.033926897 |
| P2RY13 | 0.413699743 | 0.034648964 |
| SH2D1A | 0.329491525 | 0.034651843 |
| TRAT1 | 0.606734332 | 0.034673 |
| PRKCA | 0.758287425 | 0.034746367 |
| HLA-B | 0.661596348 | 0.035422912 |
| CLEC4E | 0.28901775 | 0.035766039 |
| TBX21 | 0.255674518 | 0.036043299 |
| CD209 | 0.350046425 | 0.036091412 |
| HDAC4 | 0.752795709 | 0.036133483 |
| IFNGR1 | 1.345002621 | 0.03746272 |
| DUSP1 | 5.673509635 | 0.03753107 |
| MLANA | 0.615827838 | 0.037772371 |
| JAK2 | 1.614384166 | 0.038245047 |
| WNT11 | 0.410420475 | 0.038250608 |
| SNAI1 | 0.685438915 | 0.038591853 |
| SIRPA | 1.797329277 | 0.039285621 |
| TTC30A | 2.172297791 | 0.039657599 |
| MARCO | 0.288813559 | 0.040129841 |
| TLR5 | 1.781317662 | 0.040166627 |
| DTX4 | 0.791693202 | 0.040264004 |
| TGFBR2 | 1.374676091 | 0.040700025 |
| TNFSF13B | 0.580537415 | 0.041202454 |
| CD1C | 0.23943662 | 0.041712385 |
| RASAL1 | 0.266211074 | 0.04190487 |
| CASP1 | 2.404908196 | 0.042424052 |
| CD45RB | 0.032477781 | 0.042523592 |
| COL11A2 | 0.343700159 | 0.042971846 |
| KIR3DL1 | 0.439407289 | 0.043064497 |
| LDHB | 0.622861802 | 0.043614647 |
| TSLP | 1.495996757 | 0.044248202 |
| CDH2 | 1.352570738 | 0.044296302 |
| CXCL8 | 101.8421053 | 0.044357571 |
| TAP1 | 0.628460459 | 0.044611673 |
| CST2 | 0.318942909 | 0.045524104 |
| IFNGR2 | 1.210074949 | 0.045586462 |
| LGALS9 | 1.404910197 | 0.045771867 |
| PSMB5 | 0.828371031 | 0.045890087 |
| ESR1 | 1.221367801 | 0.046261721 |
| ANGPTL4 | 12.97788339 | 0.046427959 |
| IL11 | 2.592623755 | 0.04685132 |
| CLEC7A | 2.84097561 | 0.04754715 |
| IL6R | 2.255128205 | 0.047923778 |
| DPP4 | 0.490897477 | 0.048359994 |
| PROM1 | 0.309958308 | 0.048930816 |
| CXCL14 | 0.366289278 | 0.048933079 |
| GZMA | 0.199196787 | 0.049157233 |
| TLR8 | 1.642267406 | 0.049417515 |
| XCL1/2 | 2.141545323 | 0.04954183 |
| FGF9 | 0.327263531 | 0.05042456 |
| PRKAA2 | 1.867962466 | 0.050441792 |
| MYCT1 | 0.388950686 | 0.050443176 |
| SELL | 0.05565357 | 0.050786772 |

TABLE 14
Genes Differentially Expressed in FaDu
tumors vs RPMI-8226 tumors at baseline

| | FaDu control IgG/RPMI- | |
|---|---|---|
| Gene Name | 8226 control IgG | p-value |
| JAK3 | 0.021356544 | 6.91818E−08 |
| RASAL1 | 0.074377834 | 7.06096E−08 |
| HLA-A | 0.139358504 | 7.29427E−07 |
| RUNX3 | 0.045376221 | 7.49882E−07 |
| FADD | 5.406900666 | 2.40208E−06 |
| EZH2 | 0.399022943 | 2.58214E−06 |
| CTSS | 0.127908543 | 4.45531E−06 |
| MB21D1 | 0.468381931 | 1.10337E−05 |
| PTPRC | 0.040488987 | 1.1801E−05 |
| MAGEA1 | 0.475330815 | 1.25308E−05 |
| DNMT1 | 0.516060011 | 1.45178E−05 |
| HELLS | 1.358688351 | 1.943E−05 |
| GPR160 | 0.067262182 | 2.07067E−05 |
| KAT2B | 0.204343587 | 2.43089E−05 |
| CX3CR1 | 0.000728553 | 2.52401E−05 |
| SMAP1 | 0.403048626 | 2.53466E−05 |
| E2F3 | 0.369643241 | 2.53672E−05 |
| SLC16A1 | 2.210290916 | 2.56909E−05 |
| LGALS9 | 0.277172127 | 2.7378E−05 |
| MAML2 | 0.082407624 | 3.2451E−05 |
| ENO1 | 0.536674157 | 3.34279E−05 |
| PTCD2 | 0.553619303 | 3.90164E−05 |
| AKT1 | 1.634133094 | 4.18635E−05 |
| PECAM1 | 0.21334615 | 4.40718E−05 |
| SLC7A5 | 2.116754818 | 4.96925E−05 |
| B2M | 0.429301261 | 5.44816E−05 |
| ULBP2 | 5.155696537 | 5.92624E−05 |
| IRF3 | 0.360270481 | 6.80454E−05 |
| VEGFB | 0.198464055 | 7.54129E−05 |
| NRAS | 2.393660996 | 7.81252E−05 |
| PPARG | 10.89292929 | 8.16944E−05 |
| MKI67 | 1.7944816 | 9.43008E−05 |
| ISG15 | 3.499718624 | 9.60549E−05 |
| MS4A6A | 0.222531552 | 0.000106454 |
| ICOSLG | 0.148990617 | 0.000108638 |
| CD86 | 0.030561895 | 0.00011154 |
| GPSM3 | 0.084774249 | 0.00011366 |
| MICB | 0.393887749 | 0.000135439 |
| TYMS | 0.517667088 | 0.000140042 |
| PARP4 | 0.588638115 | 0.000140855 |
| CTNNB1 | 2.655803722 | 0.000145512 |
| ATM | 0.269294731 | 0.000152779 |
| ARNT2 | 5.547051027 | 0.000152794 |
| NFKBIA | 4.435655927 | 0.000152921 |
| MSH2 | 0.450150838 | 0.000164352 |
| IFI16 | 0.366360032 | 0.000167163 |
| FYN | 12.47525773 | 0.000182628 |
| MFNG | 0.114505591 | 0.000190196 |
| ITPK1 | 4.66902142 | 0.000204318 |
| MYC | 0.294066867 | 0.000204335 |
| MET | 25.67213999 | 0.000206393 |
| ICAM3 | 0.038195082 | 0.000210134 |
| HRAS | 2.155403551 | 0.000219663 |
| NF1 | 2.301604991 | 0.00022259 |
| VHL | 0.708840155 | 0.000227129 |
| IFNGR2 | 1.186567802 | 0.000230717 |
| PGPEP1 | 0.198353656 | 0.000237502 |
| EIF4EBP1 | 0.280140619 | 0.000238544 |
| GPC4 | 5.890821614 | 0.000238639 |
| TGFBR1 | 2.430366878 | 0.000265545 |
| BCL2 | 0.009939218 | 0.000304323 |
| TRAF1 | 0.13221359 | 0.000308593 |
| POLD1 | 0.457856637 | 0.000311719 |
| BRCA2 | 1.75796332 | 0.000320798 |
| RPL23 | 0.607182748 | 0.000332418 |
| PIK3CA | 0.452167884 | 0.000337808 |
| F2RL1 | 4.990773723 | 0.000352472 |
| BMP2 | 4.342140337 | 0.000359073 |
| PRKACB | 0.590143462 | 0.000379107 |
| TNFRSF1A | 2.910168207 | 0.000383689 |
| CBLC | 6.396601529 | 0.000396804 |
| HIF1A | 2.868054468 | 0.00040569 |
| SIRPA | 0.366263649 | 0.000409971 |
| IRF2 | 0.444180523 | 0.000411072 |
| IL18 | 10.89583001 | 0.000453732 |
| MAP3K7 | 1.405959664 | 0.000466519 |
| H2AFX | 2.700565206 | 0.00046817 |
| HLA-DMB | 0.178153302 | 0.000481052 |
| CDC20 | 1.645234155 | 0.000519841 |
| TAP2 | 0.56316117 | 0.000531963 |
| NGFR | 4.982123627 | 0.000533269 |
| HLA-DMA | 0.07321136 | 0.000536435 |
| CDKN2A | 0.550449203 | 0.000542626 |
| ANGPT1 | 0.143988802 | 0.000565604 |
| MAGEA12 | 0.096028355 | 0.000601884 |
| PRLR | 5.578724789 | 0.000612947 |
| PPARGC1B | 0.395209707 | 0.00061838 |
| ZEB1 | 0.049758353 | 0.000638327 |
| IFITM2 | 4.178901078 | 0.000639569 |
| TICAM1 | 0.550632589 | 0.000645302 |
| FBP1 | 0.332929331 | 0.000647219 |
| RRM2 | 0.390122108 | 0.000647325 |
| VCAN | 3.288006757 | 0.000650115 |
| TLR7 | 0.156285796 | 0.000668368 |
| BLK | 0.056571242 | 0.000695695 |
| TMEM140 | 0.23647917 | 0.000717748 |
| CDK6 | 0.388235392 | 0.000731952 |
| RELA | 1.627794121 | 0.000746129 |
| EIF2B4 | 0.360499334 | 0.000757204 |
| HK1 | 36.6475188 | 0.000769357 |
| THBS1 | 85.52465356 | 0.000777525 |
| ITGA4 | 0.007035957 | 0.0008225 |
| EXO1 | 0.468331355 | 0.000823689 |
| CXCR3 | 0.228061738 | 0.000831269 |
| LOXL2 | 4.030976037 | 0.000841014 |
| NOD2 | 0.188782219 | 0.000944243 |
| EDN1 | 6.49415788 | 0.000990657 |
| PSMB9 | 0.484578734 | 0.001007052 |
| NOTCH2 | 0.610883379 | 0.00100894 |
| CD276 | 3.782768769 | 0.001020907 |
| HLA-F | 0.475275565 | 0.001027004 |
| TNFRSF1B | 0.009821663 | 0.0010279 |
| CCND1 | 14.72742703 | 0.001061502 |
| CXCL12 | 0.016935936 | 0.001066419 |
| HDAC5 | 0.373765654 | 0.001201913 |
| APH1B | 5.562726801 | 0.001211157 |
| RPTOR | 1.354427465 | 0.001271206 |
| CXCR4 | 0.02212944 | 0.001292774 |
| ARG2 | 0.187054377 | 0.001298348 |
| FLNB | 2.705596239 | 0.001308627 |
| ERO1A | 2.30167062 | 0.001326453 |
| CD47 | 0.630932347 | 0.00134244 |
| MGMT | 1.870785164 | 0.001342445 |
| TNFRSF25 | 0.281127626 | 0.001344758 |
| ANLN | 2.89511925 | 0.001367048 |
| CX3CL1 | 9.370786517 | 0.001412438 |
| MAGEA4 | 25.16440049 | 0.00142051 |
| CD74 | 0.00988014 | 0.001444382 |
| TYMP | 0.154143369 | 0.001483143 |
| CASP3 | 0.680297683 | 0.001485341 |
| ICAM1 | 0.180188844 | 0.001494134 |
| PIK3CG | 0.043466523 | 0.001539056 |
| CCND2 | 0.019499151 | 0.001554274 |
| CD8B | 0.001380311 | 0.001641803 |
| NECTIN2 | 17.63707072 | 0.001648278 |
| MSH6 | 0.435118154 | 0.001673445 |
| TAPBP | 0.60620561 | 0.00170736 |
| TLR1 | 0.027235154 | 0.001746018 |
| CD70 | 0.13879824 | 0.001750492 |
| CEP55 | 1.615506955 | 0.001758374 |
| ARID1A | 0.414232291 | 0.001824989 |
| S100A9 | 21.59337427 | 0.001855197 |
| SOX11 | 0.086092306 | 0.001881515 |
| CD14 | 1.712528474 | 0.001885079 |
| TPM1 | 1.829365523 | 0.001890997 |
| CCNA1 | 30.91617273 | 0.001895511 |
| IL16 | 0.012991737 | 0.001901023 |
| HLA-B | 0.33980306 | 0.001901487 |
| BRD3 | 2.117018918 | 0.001945576 |
| COL17A1 | 152.4513587 | 0.001968259 |
| CD38 | 0.005202693 | 0.001985351 |
| SFXN1 | 1.279930211 | 0.002008855 |
| MXI1 | 0.604210694 | 0.00200923 |
| PIK3R1 | 0.237333147 | 0.002011821 |
| LILRA1 | 0.382628332 | 0.002020253 |
| GLS | 0.646858344 | 0.002026932 |
| IRF4 | 0.006322167 | 0.00202819 |
| TLR4 | 0.01266786 | 0.00216116 |
| SYK | 0.412002983 | 0.002185208 |
| IL10 | 0.043251987 | 0.002260488 |
| TSLP | 3.015526047 | 0.002266616 |
| ADORA2A | 0.12743878 | 0.002276791 |
| JAG2 | 5.01070091 | 0.002296392 |
| PSMB10 | 0.518428643 | 0.002315321 |
| IFNAR1 | 1.389830568 | 0.002334866 |
| P4HA2 | 2.17051078 | 0.002363983 |
| NECTIN1 | 5.144920357 | 0.002372739 |
| STAT4 | 0.193079991 | 0.002399924 |
| TNFRSF18 | 0.027441506 | 0.002402957 |
| TGFBR2 | 3.128431619 | 0.002409349 |
| CCR2 | 0.010962222 | 0.00256842 |
| TNFRSF17 | 0.005452873 | 0.002635249 |
| APC | 0.814959492 | 0.002646773 |
| PFKM | 0.465183919 | 0.002673124 |
| SERPINB5 | 39.13432547 | 0.002741865 |
| ITGAM | 0.463826623 | 0.002765925 |
| UBE2C | 27.01745455 | 0.002847599 |
| PRKCA | 3.53432282 | 0.002883165 |
| CD28 | 0.00485984 | 0.002884916 |
| S100A8 | 73.16790918 | 0.002886206 |
| DKK1 | 71.65295031 | 0.002907326 |
| PIK3R5 | 0.062712901 | 0.002959174 |
| LAMB3 | 3.738119234 | 0.003009933 |
| PLOD2 | 140.6481379 | 0.003026262 |
| TNFRSF10B | 3.666942875 | 0.003051394 |
| API5 | 1.218437077 | 0.003052619 |
| CYBB | 0.002753918 | 0.00312929 |
| TWF1 | 2.558127219 | 0.003133801 |
| IL7R | 89.6108156 | 0.003150236 |
| PDCD1 | 0.09093809 | 0.003196302 |
| DTX4 | 2.412419845 | 0.003227776 |
| BRD4 | 0.674164854 | 0.003232517 |
| BCAT1 | 7.753446371 | 0.003239693 |
| SLC2A1 | 1.447968705 | 0.003245796 |
| TCF3 | 0.595300126 | 0.003251543 |
| KRAS | 0.478131315 | 0.003252092 |
| FCGRT | 2.79963725 | 0.003252628 |
| CASP8 | 0.152973196 | 0.003315095 |
| BAMBI | 0.356664852 | 0.003324812 |
| TGFB1 | 0.554018981 | 0.00334258 |
| IRF5 | 0.242939721 | 0.003534386 |
| BRCA1 | 0.521528476 | 0.003598277 |
| ITGA6 | 73.91380275 | 0.003614669 |
| PC | 14.63587903 | 0.003666264 |
| IL2RG | 0.010034984 | 0.003672731 |
| TNFRSF10D | 0.150208418 | 0.003689828 |
| TPI1 | 1.355557272 | 0.003715636 |
| RB1 | 0.779479561 | 0.003737571 |
| CD79B | 0.400338314 | 0.003783598 |
| SREBF1 | 0.425545739 | 0.003919939 |
| CD58 | 4.39154044 | 0.003921491 |
| CD44 | 40.40724104 | 0.003940792 |
| FSTL3 | 4.482707326 | 0.004002343 |
| LY96 | 0.014216538 | 0.004006081 |
| DLL1 | 57.56431185 | 0.004025924 |
| CCL2 | 0.203703704 | 0.00403355 |
| PDGFB | 0.305183241 | 0.004081957 |
| BAX | 1.297754049 | 0.004150494 |
| PNOC | 0.314272359 | 0.004200882 |
| BIRC3 | 0.45519555 | 0.00428095 |
| CD45RA | 0.096993662 | 0.004335796 |
| NBN | 1.258027756 | 0.004352532 |
| HCK | 0.265643803 | 0.004421373 |
| WNT7B | 16.39162404 | 0.004433342 |
| IL1B | 19.54363545 | 0.004488802 |
| CSF2RB | 0.00982106 | 0.004620522 |
| DUSP2 | 0.1497114 | 0.004672302 |
| CDH2 | 0.081584123 | 0.004706909 |
| CTLA4 | 0.400876729 | 0.004708484 |
| NFKB1 | 1.394387502 | 0.004727308 |
| IGF2R | 0.462876814 | 0.004779385 |
| ICAM5 | 0.229724241 | 0.004832531 |
| BAD | 0.572701068 | 0.0048688 |
| FGF13 | 0.027229659 | 0.004878251 |
| OAS3 | 9.622632963 | 0.004895145 |
| IFNGR1 | 3.136458614 | 0.004919324 |
| CEBPB | 3.514425607 | 0.004953729 |
| SERPINH1 | 2.032204551 | 0.005069167 |
| BBC3 | 0.496372409 | 0.005086828 |
| HDAC4 | 0.551650621 | 0.005162981 |
| CLEC7A | 0.047680384 | 0.005204256 |
| GIMAP6 | 0.047255881 | 0.005229235 |
| EGFR | 64.79680901 | 0.005298357 |
| IL6 | 16.12855396 | 0.00530818 |
| IL6R | 0.167187161 | 0.00536172 |
| GIMAP4 | 0.013822777 | 0.005424591 |
| ERBB2 | 29.11323077 | 0.005486513 |
| GOT1 | 1.493023182 | 0.005499853 |
| ICAM2 | 0.007391005 | 0.005527934 |
| TNFRSF11A | 2.896551724 | 0.005607682 |
| IL10RA | 0.014194586 | 0.005699579 |
| PVR | 2.921513586 | 0.005704965 |
| TAPBPL | 0.539020184 | 0.005846862 |
| CD8A | 0.342897907 | 0.005867861 |
| JAG1 | 4.173530148 | 0.005946863 |
| TGFB2 | 1.706321553 | 0.005999975 |
| SLC1A5 | 0.434344842 | 0.006034058 |
| AXL | 17.86266196 | 0.006053073 |
| MTOR | 0.671301503 | 0.006093614 |
| PSMB5 | 1.83617402 | 0.006153297 |
| NFKBIE | 0.599582004 | 0.006247471 |
| CASP9 | 0.49910855 | 0.006249386 |
| NFKB2 | 0.670732794 | 0.006391467 |
| OASL | 12.79913952 | 0.00640038 |
| TNFRSF14 | 0.120661205 | 0.006598264 |
| HMGB1 | 5.810318995 | 0.00666793 |
| HLA-DPA1 | 0.047516892 | 0.006668383 |
| TLR9 | 0.021113302 | 0.006823674 |
| MAP3K8 | 0.617277059 | 0.006902614 |
| RAD51C | 0.704226118 | 0.006905648 |
| CD300A | 0.01303607 | 0.006941955 |
| CCNE1 | 0.732404343 | 0.006950107 |
| ITGB8 | 36.2275198 | 0.006970622 |
| RICTOR | 1.223319078 | 0.007091008 |
| STAT1 | 1.606954632 | 0.007202677 |
| BN1P3L | 1.546202218 | 0.007508898 |
| CD6 | 0.149263552 | 0.00752 |
| HES1 | 12.06296354 | 0.007537324 |
| PMS2 | 0.716559489 | 0.007537954 |
| RAD51 | 0.713858203 | 0.007558913 |
| C1QA | 0.247471857 | 0.007636568 |
| PROM1 | 0.745642881 | 0.007691823 |
| CDH1 | 3.273869801 | 0.007816375 |
| CTAG1B | 0.017166572 | 0.007981098 |
| PDK1 | 0.737652262 | 0.008166862 |
| EOMES | 10.47650602 | 0.008167402 |
| LAIR1 | 0.011338364 | 0.008233933 |
| CHUK | 1.533607197 | 0.00831615 |
| ITGA2 | 28.83968314 | 0.008366596 |
| BCL2L1 | 0.514483697 | 0.00860537 |
| MAP3K5 | 0.238605898 | 0.008929537 |
| NFATC2 | 0.30146283 | 0.00894379 |
| MMP1 | 97.53985507 | 0.008986547 |
| FCGR2A | 0.011951954 | 0.009018087 |
| HLA-DRA | 0.033708861 | 0.00915853 |
| MMP7 | 4.618128868 | 0.009172315 |
| CSF1 | 4.090464224 | 0.009242476 |
| IFI27 | 12.07825108 | 0.009629522 |
| BIRC5 | 1.487884734 | 0.009643019 |
| HEY1 | 0.185743243 | 0.009734687 |
| FZD8 | 2.632303412 | 0.009795842 |
| WNT10A | 1.833364163 | 0.009950554 |
| VEGFC | 25.26179394 | 0.009984065 |
| RNLS | 2.721978022 | 0.010016913 |
| ELOB | 0.491934909 | 0.010172203 |
| CD274 | 3.2124197 | 0.010227045 |
| SAMD9 | 2.967461557 | 0.010282678 |
| PRR5 | 1.973275738 | 0.010329223 |
| IRF9 | 3.71296349 | 0.010344489 |
| HAVCR2 | 0.008043445 | 0.010507998 |
| CD48 | 0.110222692 | 0.010646136 |
| FCGR2B | 0.214161749 | 0.010713342 |
| TAP1 | 0.630023529 | 0.011146193 |
| TNFRSF8 | 0.084826224 | 0.011148784 |
| TMEM173 | 0.70925644 | 0.01120653 |
| CRABP2 | 5.484583105 | 0.011238381 |
| CDKN1A | 2.877865663 | 0.011389383 |
| CXCL1 | 45.24471126 | 0.011538065 |
| SELE | 0.511039743 | 0.011561658 |
| PDCD1LG2 | 1.852554395 | 0.011793128 |
| DUSP5 | 0.571389135 | 0.011928487 |
| PRKAA2 | 2.799598192 | 0.012022709 |
| NDUFA4L2 | 0.722566183 | 0.012084867 |
| CCL22 | 1.947001065 | 0.012442799 |
| RBL2 | 0.787075625 | 0.012761051 |
| CD3E | 0.410183876 | 0.012818147 |
| IL1A | 58.14295657 | 0.012840822 |
| BBS1 | 1.397787067 | 0.01316386 |
| RORC | 0.218897936 | 0.01321063 |
| OLFML2B | 0.042802041 | 0.013392627 |
| IFIT2 | 54.2407297 | 0.013495884 |
| SPP1 | 0.000126682 | 0.013691983 |
| MRC1 | 0.447338185 | 0.013706802 |
| GBP1 | 5.035416929 | 0.013729336 |
| C2 | 0.211464355 | 0.013826776 |
| IKBKG | 0.576796702 | 0.013854882 |
| NLRC5 | 0.538315736 | 0.013990295 |
| IL1RN | 2.723557399 | 0.014547679 |
| GMIP | 0.316964286 | 0.014798024 |
| DEPTOR | 0.046913056 | 0.014812096 |
| CES3 | 0.599681746 | 0.014830429 |
| IFIT3 | 6.946190364 | 0.014842148 |
| TPSAB1/B2 | 0.407200964 | 0.015028194 |
| FOSL1 | 30.45099034 | 0.015099046 |
| IL21R | 0.166242967 | 0.015364512 |
| IFIT1 | 8.011607104 | 0.015465292 |
| MAGEA3/A6 | 0.065278837 | 0.015674362 |
| TLR2 | 6.618221734 | 0.0158337 |
| TGFB3 | 1.988054116 | 0.016152723 |
| IRF8 | 0.041744665 | 0.016211243 |
| EPM2AIP1 | 0.869528497 | 0.016314987 |
| TNFSF12 | 4.143891403 | 0.01663901 |
| LY9 | 0.025925048 | 0.016742823 |
| JAK2 | 3.680546429 | 0.016749941 |
| LILRB4 | 0.024172359 | 0.01691062 |
| CCL20 | 27.85772084 | 0.017057344 |
| CCL8 | 0.434927198 | 0.017225662 |
| CXCL9 | 0.01720115 | 0.017241116 |
| CXCL2 | 83.83700178 | 0.017281009 |
| LAMC2 | 99.64753053 | 0.017461826 |
| MRE11 | 0.785073755 | 0.017472249 |
| CMKLR1 | 0.129779968 | 0.017484503 |
| TNFSF8 | 0.37375981 | 0.017665874 |
| EGR1 | 20.05942866 | 0.017776485 |
| PKM | 0.604324865 | 0.017920734 |
| TLR5 | 3.206570156 | 0.018143551 |
| CXCL10 | 0.066359099 | 0.018234706 |
| TLR8 | 2.515952426 | 0.018589078 |
| MAGEB2 | 0.447938191 | 0.018692518 |
| MMRN2 | 2.294974359 | 0.018761767 |
| PSMB8 | 0.738024175 | 0.018860819 |
| ZEB2 | 0.604402935 | 0.018995162 |
| RIPK3 | 0.437296192 | 0.019233057 |
| HERC6 | 8.723829885 | 0.019244154 |
| ZC3H12A | 1.961591816 | 0.019325938 |
| CDKN2B | 2.403344917 | 0.019731259 |
| UBE2T | 0.726931366 | 0.019736616 |
| IFITM1 | 1.832666816 | 0.020027714 |
| CD45RO | 0.236597938 | 0.020102117 |
| TLR3 | 9.746698065 | 0.020917995 |
| WNT5B | 0.478065993 | 0.021199226 |
| XCL1/2 | 3.437565803 | 0.0213437 |
| LDHA | 0.783222199 | 0.021806921 |
| ITGAL | 0.143421885 | 0.021995976 |
| WNT5A | 0.16215526 | 0.022016648 |
| SLAMF7 | 0.284173597 | 0.02204568 |
| TP53 | 0.890627695 | 0.022173575 |
| OLR1 | 14.96769606 | 0.02221587 |
| CD40 | 0.289078255 | 0.022462659 |
| RASGRF1 | 1.713318734 | 0.02262894 |
| RSAD2 | 9.955867414 | 0.02275374 |
| KLRD1 | 0.529801325 | 0.022994728 |
| CDKN1C | 0.202667621 | 0.023617012 |
| THBD | 4.017651573 | 0.023626005 |
| HK2 | 1.595849165 | 0.023644425 |
| PVRIG | 0.679836242 | 0.023714955 |
| COL4A5 | 17.59217687 | 0.023763682 |
| MS4A4A | 0.052547439 | 0.023866864 |
| SOX2 | 2.404076739 | 0.02476937 |
| STC1 | 21.42696011 | 0.024821828 |
| CD45RB | 0.098585524 | 0.024854966 |
| NOTCH1 | 2.108804004 | 0.02492947 |
| LIF | 32.23809524 | 0.024932087 |
| AREG | 94.41523886 | 0.025373332 |
| FUT4 | 1.549951267 | 0.025562585 |
| CCL19 | 0.661263347 | 0.025724938 |
| CXCL3 | 2.951440636 | 0.025981771 |
| IRF7 | 1.775355384 | 0.026077505 |
| HLA-DRB1 | 0.089639849 | 0.026126932 |
| SAMSN1 | 0.019814956 | 0.026223818 |
| LTBP1 | 0.38205544 | 0.02628653 |
| IDO1 | 0.650385604 | 0.026611394 |
| TNFSF4 | 0.11611899 | 0.027165126 |
| CCL3/L1 | 0.148096461 | 0.027716078 |
| LILRB2 | 0.230231629 | 0.027789061 |
| FAS | 3.138068416 | 0.027892666 |
| TNFSF9 | 0.568592058 | 0.028828192 |
| ITGAV | 1.696931191 | 0.029292287 |
| WNT2B | 2.845568657 | 0.029431331 |
| PARP9 | 1.879528025 | 0.030234712 |
| SERPINA1 | 1.939930194 | 0.030389832 |
| GZMM | 0.50767583 | 0.030593968 |
| CD247 | 0.539617205 | 0.030681196 |
| IL24 | 2.501776295 | 0.03076744 |
| PTEN | 0.733009806 | 0.030918922 |
| MAGEC2 | 0.074205292 | 0.031603408 |
| TNFAIP6 | 2.934493347 | 0.032384309 |
| IER3 | 9.468527706 | 0.032413486 |
| BNIP3 | 1.502404865 | 0.032511553 |
| TNFAIP3 | 6.929816194 | 0.032514249 |
| IFI6 | 2.169370011 | 0.03274536 |
| CXCL6 | 0.095733484 | 0.032768797 |
| HDAC11 | 1.423474064 | 0.032818786 |
| NCAM1 | 0.03072863 | 0.033307507 |
| FGF9 | 0.204768435 | 0.033460905 |
| COL11A2 | 0.516064658 | 0.03353656 |
| SGK1 | 1.907354728 | 0.034178577 |
| MELK | 1.162804718 | 0.034559647 |
| AXIN1 | 0.770430637 | 0.034648533 |
| CD68 | 0.699121464 | 0.034681554 |
| IL4 | 0.497279196 | 0.034779317 |
| DAB2 | 1.253872863 | 0.035372955 |
| ZAP70 | 0.179886686 | 0.03546089 |
| MS4A1 | 0.076766381 | 0.035757299 |
| DEFB134 | 0.667543201 | 0.036008515 |
| FCAR | 0.48849327 | 0.037169891 |
| GLUL | 1.788942914 | 0.037305038 |
| IL11 | 3.040789474 | 0.037825804 |
| MFGE8 | 0.550937207 | 0.038716283 |
| PIK3CD | 0.456044181 | 0.039037181 |
| HLA-DOB | 0.46041602 | 0.039225219 |
| IFIH1 | 0.692316535 | 0.039459978 |
| FCRL2 | 0.135958152 | 0.039657656 |
| CD84 | 0.18017847 | 0.039832967 |
| VEGFA | 1.409913077 | 0.039944107 |
| IL15 | 1.703072421 | 0.039961603 |
| IKBKB | 1.576069353 | 0.040068667 |
| NID2 | 0.702575798 | 0.040315919 |
| COL5A1 | 1.883993464 | 0.042622079 |
| FGFR1 | 0.663685965 | 0.043491757 |
| HDAC3 | 1.331031138 | 0.043522729 |
| SMAD5 | 0.787917126 | 0.043667604 |
| FCGR1A | 0.112060998 | 0.043796837 |
| ANGPTL4 | 16.58537725 | 0.044866931 |
| KIT | 0.040008395 | 0.04580592 |
| FCN1 | 0.692426401 | 0.046315574 |
| SIGLEC8 | 0.645459721 | 0.047266688 |
| TBXAS1 | 0.239709643 | 0.047327715 |
| RELN | 0.405496625 | 0.047509092 |
| CXCL8 | 21.04770965 | 0.047668898 |
| CD3G | 0.450515217 | 0.047963958 |
| DTX3L | 1.598617616 | 0.048525451 |
| UBA7 | 2.110962396 | 0.048669918 |
| BLM | 0.829341038 | 0.048688456 |
| FLT1 | 2.276127321 | 0.048870719 |
| TRIM21 | 1.663330118 | 0.049492806 |
| PIAS4 | 0.746021139 | 0.049523675 |
| CD5 | 0.328636552 | 0.050250437 |
| TDO2 | 0.561917593 | 0.050503849 |
| STAT3 | 1.238098668 | 0.050972411 |

TABLE 15
Genes Differentially Expressed in H1975 immune infiltrates
vs Raji immune infiltrates at baseline

| | H1975 control IgG/ | |
|---|---|---|
| Gene Name | Raji control IgG | p-value |
| Rnase2b | 0.242233 | 0.000005 |
| Ccr4 | 0.120263 | 0.000014 |
| Itgam | 0.157314 | 0.000036 |
| Nfil3 | 0.255383 | 0.000046 |
| Tnfaip3 | 0.054600 | 0.000064 |
| Nox1 | 5.018713 | 0.000067 |
| Pfdn6 | 3.936779 | 0.000076 |
| Ccl2 | 0.172905 | 0.000121 |
| Ptgdr | 0.208159 | 0.000127 |
| Klf10 | 0.455844 | 0.000145 |
| Tnfaip8 | 0.208543 | 0.000153 |
| Cd28 | 4.390562 | 0.000162 |
| Tgm2 | 0.245075 | 0.000174 |
| Aoah | 0.087406 | 0.000200 |
| Rictor | 0.316662 | 0.000203 |
| Ccr7 | 0.070031 | 0.000251 |
| Lgals3 | 0.150129 | 0.000260 |
| Gtf2h2 | 3.818177 | 0.000287 |
| Ccl9 | 0.109250 | 0.000292 |
| Mafb | 0.253124 | 0.000371 |
| Yap1 | 2.666263 | 0.000391 |
| Ccl24 | 0.235350 | 0.000411 |
| Stat5a | 0.587714 | 0.000424 |
| Cdkn1a | 0.241985 | 0.000425 |
| Eif4ebp2 | 0.470642 | 0.000491 |
| 2810417H13Rik | 3.485619 | 0.000495 |
| Ccnb2 | 2.833720 | 0.000537 |
| Tspan7 | 0.390920 | 0.000565 |
| Tnfsf9 | 0.176323 | 0.000573 |
| Tnfrsf14 | 0.247605 | 0.000578 |
| Ptafr | 0.123758 | 0.000621 |
| Cd80 | 0.132482 | 0.000676 |
| Ifnar1 | 0.360519 | 0.000709 |
| Prdx3 | 1.653359 | 0.000714 |
| Rgl1 | 0.251979 | 0.000734 |
| C5ar1 | 0.207702 | 0.000790 |
| Ccr5 | 0.335303 | 0.000839 |
| Il4i1 | 0.056369 | 0.000869 |
| Ccr2 | 0.180246 | 0.000877 |
| Cstb | 0.239561 | 0.000894 |
| Irf1 | 0.189128 | 0.000897 |
| Clec5a | 0.240799 | 0.000904 |
| Ccl22 | 0.110117 | 0.001078 |
| Klk1 | 0.310336 | 0.001127 |
| Map3k4 | 3.925148 | 0.001155 |
| Acod1 | 0.179580 | 0.001209 |
| Id2 | 0.214039 | 0.001225 |
| Cd68 | 0.245842 | 0.001226 |
| S100a4 | 0.154630 | 0.001291 |
| Arg1 | 0.077485 | 0.001326 |
| Cav1 | 0.238382 | 0.001380 |
| Batf3 | 0.155304 | 0.001410 |
| Maff | 0.210437 | 0.001515 |
| Vamp2 | 0.138408 | 0.001520 |
| Twistnb | 2.826299 | 0.001625 |
| Ms4a1 | 0.031820 | 0.001686 |
| Mmp19 | 0.310322 | 0.001741 |
| Nfkbiz | 0.435417 | 0.001839 |
| Fcgr2b | 0.169951 | 0.001881 |
| Prg2 | 0.251708 | 0.002038 |
| Nfkbia | 0.222760 | 0.002043 |
| Cd274 | 0.185308 | 0.002063 |
| Il1a | 0.075467 | 0.002081 |
| Erg | 0.331732 | 0.002141 |
| Adgre5 | 0.257757 | 0.002270 |
| Rhog | 0.367090 | 0.002287 |
| Hif1a | 0.405290 | 0.002364 |
| Yes1 | 3.526137 | 0.002513 |
| Irf8 | 0.300793 | 0.002530 |
| Fzd6 | 0.760728 | 0.002563 |
| Tlr5 | 0.110357 | 0.002583 |
| Ptprc | 0.289859 | 0.002648 |
| Nox3 | 0.276093 | 0.002692 |
| Il1rn | 0.039417 | 0.002737 |
| Hivep1 | 0.355638 | 0.002755 |
| Csf1r | 0.295373 | 0.002763 |
| Fbxl7 | 0.420801 | 0.002801 |
| Ear6 | 0.246939 | 0.002841 |
| Fem1c | 0.433836 | 0.002882 |
| Fyn | 0.162359 | 0.002892 |
| Flt3 | 0.326475 | 0.002955 |
| Rad51 | 0.351197 | 0.002959 |
| Fcgr3 | 0.257958 | 0.002987 |
| Ell2 | 4.181589 | 0.003084 |
| Marcksl1 | 0.092696 | 0.003090 |
| Tlr8 | 0.305374 | 0.003183 |
| Zfp148 | 0.243157 | 0.003189 |
| Cebpb | 0.210440 | 0.003225 |
| Map2k3 | 0.298906 | 0.003226 |
| Ndufa7 | 0.625140 | 0.003233 |
| Ifnar2 | 0.387098 | 0.003291 |
| Col4a2 | 0.575436 | 0.003328 |
| H2-D1 | 0.313357 | 0.003439 |
| Nod2 | 0.165871 | 0.003516 |
| Angpt2 | 0.256789 | 0.003553 |
| Il1rl1 | 0.259863 | 0.003581 |
| Psme2 | 0.138131 | 0.003713 |
| H2-Q1 | 0.382562 | 0.003720 |
| Csf2ra | 0.249821 | 0.003757 |
| Selp | 0.116613 | 0.003775 |
| Ccr1 | 0.170646 | 0.004195 |
| Kif20a | 5.560170 | 0.004206 |
| Cebpd | 0.285088 | 0.004217 |
| Tek | 0.503457 | 0.004234 |
| Ifngr1 | 0.263915 | 0.004426 |
| Mmp9 | 0.312285 | 0.004627 |
| Vasp | 0.322755 | 0.004823 |
| Il6ra | 0.390894 | 0.004844 |
| Laptm5 | 0.393519 | 0.004886 |
| Mcm5 | 5.918278 | 0.004899 |
| Ier3 | 0.229260 | 0.004929 |
| Marco | 0.008050 | 0.004959 |
| Il5 | 0.132653 | 0.004995 |
| Des | 0.049585 | 0.005256 |
| Sema4a | 0.093883 | 0.005285 |
| Tpsb2 | 0.271684 | 0.005298 |
| Nr4a1 | 0.273722 | 0.005310 |
| Armc1 | 1.681844 | 0.005321 |
| Txn1 | 1.711753 | 0.005328 |
| Sele | 0.109975 | 0.005360 |
| Tyrobp | 0.294246 | 0.005373 |
| Cd38 | 0.107777 | 0.005532 |
| Stat3 | 0.591871 | 0.005539 |
| Malt1 | 0.311922 | 0.005561 |
| Vsir | 0.418730 | 0.005587 |
| Tuba4a | 4.651330 | 0.005741 |
| H2-DMb2 | 0.135821 | 0.005771 |
| F11r | 0.435258 | 0.005785 |
| Skil | 0.715657 | 0.005871 |
| Ikzf1 | 2.195265 | 0.005875 |
| Cd84 | 0.281488 | 0.006013 |
| Cxcl16 | 0.258664 | 0.006020 |
| Il2 | 0.411293 | 0.006046 |
| Csf2 | 0.109657 | 0.006058 |
| Ccrl2 | 0.081021 | 0.006154 |
| Areg | 0.248976 | 0.006216 |
| Pggt1b | 0.557523 | 0.006297 |
| Lamp3 | 0.246561 | 0.006927 |
| Fosb | 0.146583 | 0.007195 |
| Ncf2 | 0.264462 | 0.007230 |
| Thbd | 0.288241 | 0.007242 |
| Prg3 | 0.094666 | 0.007332 |
| Stat6 | 0.453737 | 0.007622 |
| Nfyc | 0.302832 | 0.007715 |
| Vav1 | 0.486723 | 0.007720 |
| Mapk12 | 0.391989 | 0.007721 |
| Cd86 | 0.409055 | 0.008072 |
| Lat2 | 0.511459 | 0.008106 |
| Cyp3a13 | 0.169765 | 0.008126 |
| Cd83 | 0.334726 | 0.008169 |
| Ccr6 | 0.536719 | 0.008194 |
| Hist2h2aa1 | 5.929416 | 0.008472 |
| Cd14 | 0.069464 | 0.008508 |
| Cd44 | 0.459286 | 0.008658 |
| Acly | 1.641297 | 0.008759 |
| Rnase2a | 0.152118 | 0.009142 |
| Btla | 0.181142 | 0.009148 |
| Pglyrp1 | 0.095082 | 0.009175 |
| Nos2 | 0.101990 | 0.009176 |
| Ctss | 0.478490 | 0.009354 |
| Cdh4 | 0.164766 | 0.009437 |
| Peli1 | 0.110999 | 0.009457 |
| Sirpa | 0.170275 | 0.009470 |
| Top2a | 4.860298 | 0.009696 |
| Pbx3 | 3.028747 | 0.009879 |
| Tlr13 | 0.384141 | 0.009915 |
| Ccl3 | 0.114428 | 0.010067 |
| Alox5ap | 0.322499 | 0.010121 |
| Tnfaip6 | 0.189924 | 0.010137 |
| Icam1 | 0.127739 | 0.010139 |
| Cd99 | 0.260547 | 0.010144 |
| Il10ra | 0.359941 | 0.010273 |
| Crabp2 | 2.738927 | 0.010372 |
| Clec7a | 0.087083 | 0.010663 |
| Cxcl13 | 0.018367 | 0.010681 |
| Cnn2 | 0.246648 | 0.010748 |
| C3ar1 | 0.372734 | 0.010797 |
| Batf | 0.137241 | 0.010814 |
| Smad1 | 0.489142 | 0.010821 |
| Cldn1 | 0.205414 | 0.010844 |
| Il21r | 0.348941 | 0.010847 |
| Atf3 | 0.198397 | 0.011193 |
| Cd40 | 0.223573 | 0.011217 |
| Fscn1 | 0.427736 | 0.011266 |
| Birc2 | 0.443071 | 0.011309 |
| Il1b | 0.051465 | 0.011346 |
| Hist1h1c | 4.568969 | 0.011361 |
| S1pr1 | 0.303393 | 0.011381 |
| Timd4 | 0.161279 | 0.011407 |
| Fas | 0.132705 | 0.011551 |
| Slc16a6 | 0.419191 | 0.011635 |
| Il33 | 0.320081 | 0.011678 |
| Acot11 | 0.310851 | 0.011679 |
| Cdh5 | 0.556639 | 0.011735 |
| Bcl2 | 0.033445 | 0.011921 |
| Tnfrsf11a | 0.424326 | 0.012159 |
| Adam8 | 0.111380 | 0.012172 |
| H2-K1 | 0.379048 | 0.012232 |
| Nr1h3 | 0.072125 | 0.012270 |
| Klf4 | 0.358734 | 0.012496 |
| Itga4 | 0.323285 | 0.012712 |
| Tnf | 0.258142 | 0.012858 |
| Csf3 | 0.106341 | 0.012878 |
| Mmp1a | 0.159709 | 0.012990 |
| Itgb2 | 0.233839 | 0.013293 |
| Ttk | 2.914750 | 0.013307 |
| Serpine3 | 0.314853 | 0.013335 |
| Gch1 | 0.267172 | 0.013470 |
| Cxcl5 | 0.275873 | 0.013550 |
| Hoxd4 | 0.520961 | 0.013892 |
| Tlr9 | 0.324242 | 0.013982 |
| Tlr2 | 0.142900 | 0.014418 |
| Il18r1 | 0.191176 | 0.014507 |
| C3 | 0.099500 | 0.014563 |
| Igf2 | 0.171258 | 0.014621 |
| Cel12 | 0.473715 | 0.014707 |
| Fasn | 4.672687 | 0.014897 |
| Fcgr1 | 0.518109 | 0.015351 |
| Cd1d1 | 1.582386 | 0.015440 |
| Enpp2 | 0.023968 | 0.015481 |
| Rgs16 | 0.463805 | 0.015493 |
| Msh2 | 5.213708 | 0.015677 |
| Cbr1 | 2.014828 | 0.015728 |
| Mrc1 | 0.295738 | 0.015956 |
| Il27ra | 0.103826 | 0.016034 |
| Cyp1b1 | 0.290433 | 0.016057 |
| Col4a1 | 0.699588 | 0.016169 |
| Ccl6 | 0.249865 | 0.016185 |
| Cd70 | 0.221823 | 0.016274 |
| Serpinb2 | 0.027599 | 0.016283 |
| Traf1 | 0.065502 | 0.016664 |
| Insig1 | 2.971816 | 0.016729 |
| Acox1 | 0.459835 | 0.017159 |
| Itgal | 0.345207 | 0.017479 |
| Daxx | 0.184953 | 0.017614 |
| Tlr4 | 0.339135 | 0.017640 |
| Acot3 | 0.125498 | 0.018052 |
| Ptx3 | 0.388363 | 0.018127 |
| Dusp1 | 0.140478 | 0.018494 |
| Adamts3 | 0.270447 | 0.018551 |
| Gpr65 | 0.373650 | 0.018552 |
| Mmp12 | 0.052645 | 0.018689 |
| Serpine1 | 0.481846 | 0.018696 |
| Faf1 | 0.625184 | 0.019339 |
| Ccl26 | 0.176653 | 0.019377 |
| Cd47 | 0.455048 | 0.019378 |
| S100a10 | 0.463751 | 0.019494 |
| Selplg | 0.531978 | 0.019572 |
| Nkg7 | 0.172662 | 0.019720 |
| Csf3r | 0.177468 | 0.019721 |
| Sp4 | 0.685864 | 0.019730 |
| Met | 3.415005 | 0.020238 |
| Ets1 | 1.287413 | 0.020629 |
| Fcnb | 0.194408 | 0.020678 |
| Grn | 0.555410 | 0.020731 |
| Birc5 | 1.492233 | 0.020737 |
| Ikbke | 0.132605 | 0.020775 |
| Fabp4 | 0.099928 | 0.020844 |
| Gata2 | 0.411592 | 0.021037 |
| Cd74 | 0.211690 | 0.021038 |
| Sesn1 | 0.177741 | 0.021421 |
| Dhrs3 | 0.484168 | 0.021436 |
| Cxcr5 | 0.185337 | 0.021726 |
| Rbp1 | 0.216065 | 0.021942 |
| Ctsl | 0.334727 | 0.022009 |
| Il10 | 4.699225 | 0.022310 |
| Lst1 | 0.263043 | 0.022503 |
| Smarcd3 | 0.101035 | 0.022512 |
| Itgb7 | 0.293812 | 0.022801 |
| Nfkb1 | 0.661356 | 0.022878 |
| Alcam | 0.444914 | 0.023082 |
| Tnfrsf1b | 0.227106 | 0.023113 |
| Cysltr1 | 0.257110 | 0.023507 |
| Crem | 0.243719 | 0.023547 |
| Tap1 | 0.687186 | 0.024098 |
| Irf4 | 17.963797 | 0.024336 |
| Mertk | 0.390574 | 0.024461 |
| Cx3cr1 | 8.311072 | 0.024463 |
| Anxa4 | 0.303347 | 0.024538 |
| Tigit | 0.248739 | 0.024728 |
| Nfkbie | 0.093522 | 0.024793 |
| S100a9 | 0.105020 | 0.024835 |
| Fpr-rs4 | 0.052929 | 0.024926 |
| Trim9 | 0.147698 | 0.025039 |
| Jun | 2.004713 | 0.025053 |
| Chil3 | 0.379882 | 0.025288 |
| Chil4 | 0.270459 | 0.025637 |
| Vcam1 | 0.109494 | 0.025843 |
| Myc | 6.778348 | 0.025899 |
| Havcr2 | 0.531882 | 0.026258 |
| Egr3 | 0.220949 | 0.026471 |
| Ptgdr2 | 0.104310 | 0.026544 |
| Pdgfb | 0.523153 | 0.026733 |
| Pbx1 | 2.809932 | 0.026735 |
| H2-Ab1 | 0.192621 | 0.026852 |
| Map2k1 | 1.257664 | 0.027143 |
| Adcyap1r1 | 0.139699 | 0.027299 |
| Rab20 | 0.387463 | 0.027382 |
| Fpr-rs6 | 0.184519 | 0.027421 |
| Lamb3 | 0.065240 | 0.028162 |
| H2-Aa | 0.197630 | 0.028259 |
| Trem1 | 0.051854 | 0.028513 |
| Cdh13 | 0.370439 | 0.028835 |
| Ms4a2 | 0.333544 | 0.028857 |
| Rnd3 | 3.108069 | 0.029034 |
| Ptgs2 | 0.397200 | 0.029142 |
| Il23a | 0.203342 | 0.029618 |
| Olr1 | 0.272356 | 0.029766 |
| Dnajb6 | 0.523398 | 0.029899 |
| C1qa | 0.510834 | 0.030002 |
| Fpr-rs5 | 0.034068 | 0.030057 |
| H2-Eb1 | 0.179418 | 0.030394 |
| Adamts14 | 0.489270 | 0.030417 |
| Pf4 | 0.057102 | 0.030665 |
| Cybb | 0.241795 | 0.030790 |
| Tlr6 | 0.301169 | 0.030850 |
| Cd247 | 0.200510 | 0.031004 |
| Adora2a | 0.216708 | 0.031537 |
| Ndc80 | 1.610530 | 0.031826 |
| Kitl | 0.451793 | 0.031890 |
| Alox5 | 0.316802 | 0.032124 |
| Ctgf | 6.713420 | 0.032310 |
| Map2k4 | 1.374239 | 0.032343 |
| Icos | 0.182362 | 0.032876 |
| Col12a1 | 9.167768 | 0.032996 |
| Amica1 | 0.568566 | 0.033100 |
| Gpr183 | 0.335105 | 0.033156 |
| Cd36 | 0.085920 | 0.033206 |
| Ccl5 | 0.063108 | 0.033268 |
| Tnc | 6.185290 | 0.033314 |
| Gem | 0.191019 | 0.033613 |
| Sell | 0.076174 | 0.033713 |
| Was | 0.364785 | 0.034195 |
| C1qb | 0.539716 | 0.034515 |
| Tlr11 | 0.138966 | 0.034677 |
| Havcr1 | 0.153709 | 0.034796 |
| Camp | 0.150217 | 0.035155 |
| Psmb9 | 0.420197 | 0.035462 |
| Zmpste24 | 0.413088 | 0.035826 |
| TnfrsfS | 0.211296 | 0.035863 |
| Syk | 0.596861 | 0.036014 |
| Hnf1b | 0.273178 | 0.036102 |
| Adamts17 | 0.505122 | 0.036924 |
| Aif1 | 0.238855 | 0.037020 |
| Btg2 | 4.116198 | 0.037245 |
| Ctsd | 0.505686 | 0.037377 |
| Il1r2 | 0.110239 | 0.037673 |
| S100a8 | 0.067184 | 0.038821 |
| Ncor1 | 1.377778 | 0.039395 |
| He | 0.186667 | 0.039467 |
| Cxcr4 | 0.627046 | 0.039686 |
| Csf1 | 0.394390 | 0.039932 |
| Edn1 | 0.378740 | 0.040192 |
| Tgfb1 | 0.570695 | 0.040214 |
| Gata6 | 0.416022 | 0.040294 |
| Cd34 | 0.540273 | 0.041022 |
| Nampt | 0.705911 | 0.041128 |
| Anxa1 | 0.552374 | 0.041453 |
| Tspan8 | 0.291661 | 0.042245 |
| Serpinb9 | 0.468627 | 0.042863 |
| Hmgb1 | 1.356982 | 0.043118 |
| Nlrp3 | 0.112150 | 0.043145 |
| Il15ra | 0.536019 | 0.043274 |
| Il18 | 0.162720 | 0.043369 |
| Pdzk1ip1 | 0.270717 | 0.043702 |
| Col11a1 | 9.476761 | 0.043719 |
| Ptpn14 | 0.251194 | 0.043797 |
| Tap2 | 0.465800 | 0.044026 |
| Cnksr1 | 0.203938 | 0.044161 |
| Fosl1 | 0.304797 | 0.044425 |
| Cel19 | 0.022238 | 0.044852 |
| Prkci | 1.560791 | 0.044977 |
| Btk | 0.418923 | 0.045933 |
| Cebpa | 0.502332 | 0.046301 |
| Serinc2 | 2.003640 | 0.046371 |
| Cxcl11 | 0.220291 | 0.046619 |
| Socs2 | 1.642912 | 0.047003 |
| Flnb | 0.636359 | 0.047110 |
| Nod1 | 0.358624 | 0.047205 |
| Cd151 | 0.565169 | 0.047284 |
| Cxcl1 | 0.212880 | 0.047284 |
| Flt1 | 0.542062 | 0.047570 |
| Birc3 | 0.453764 | 0.047984 |
| Ptprb | 0.545002 | 0.048492 |
| Tgfa | 0.574790 | 0.048695 |
| Pparg | 0.170172 | 0.049111 |
| Ltb4rl | 0.143405 | 0.049274 |
| Map2k2 | 1.153432 | 0.049340 |
| Cxcl3 | 0.069406 | 0.049981 |
| Cxcl9 | 0.438896 | 0.050145 |
| Cel11 | 0.247518 | 0.050361 |
| Dpp4 | 0.364947 | 0.050467 |

TABLE 16
Genes Differentially Expressed in H1975 immune infiltrates
vs RPMI-8226 immune infiltrates at baseline.

| | H1975 control IgG/ | |
|---|---|---|
| Gene Name | RPMI-8226 control IgG | p-value |
| Tnfaip8 | 0.112030014 | 6.07008E−06 |
| Pggt1b | 0.201773241 | 3.70394E−05 |
| Col4a1 | 0.250912837 | 4.0398E−05 |
| Acly | 2.688678683 | 5.39024E−05 |
| Nox1 | 6.115684093 | 5.80953E−05 |
| Peli1 | 0.117596001 | 7.52395E−05 |
| Irf1 | 0.228024866 | 8.64092E−05 |
| Col15a1 | 0.268340311 | 9.94753E−05 |
| Tgm2 | 0.218488976 | 0.000106829 |
| Aifl | 0.10774578 | 0.000115102 |
| Cd68 | 0.167184641 | 0.000153025 |
| Lipa | 0.312047267 | 0.000174166 |
| Timd4 | 0.183104895 | 0.000176208 |
| Cdkn1a | 0.425174282 | 0.000206895 |
| Sesn1 | 0.170646158 | 0.000258095 |
| Dnajb6 | 0.474795129 | 0.000280406 |
| Ifit1bl2 | 0.247126437 | 0.000310734 |
| Socs2 | 2.165508388 | 0.00042276 |
| Smad7 | 0.174919109 | 0.000431159 |
| Pde4a | 0.39815258 | 0.000463777 |
| Ctsd | 0.263415082 | 0.000485629 |
| Cav1 | 0.277841072 | 0.000489411 |
| Mertk | 0.189385704 | 0.000491654 |
| Col4a2 | 0.208904316 | 0.000496935 |
| Pfdn6 | 3.945093039 | 0.000501441 |
| Fzd9 | 0.148170547 | 0.000537577 |
| Was | 0.300059382 | 0.000566943 |
| Hdc | 0.273013295 | 0.000663295 |
| C1qb | 0.280185948 | 0.000869184 |
| Crem | 0.254716087 | 0.000889683 |
| Zmpste24 | 0.199460554 | 0.00091815 |
| Lrp6 | 0.348711838 | 0.000940952 |
| Phlda2 | 0.417404859 | 0.000988817 |
| Fcgr3 | 0.304870278 | 0.001067935 |
| Tlr8 | 0.279018657 | 0.001102372 |
| Twistnb | 3.624299542 | 0.001248066 |
| Il18r1 | 0.128045619 | 0.001264675 |
| Cd38 | 0.245482749 | 0.001362191 |
| Cd99 | 0.192513369 | 0.001381374 |
| Batf3 | 0.175040872 | 0.001432333 |
| H2-Q1 | 0.338719804 | 0.001455924 |
| Cxcr5 | 0.161532133 | 0.001482396 |
| Fzd4 | 0.394857601 | 0.001489915 |
| Cxcl12 | 0.082928141 | 0.001535383 |
| Cdh4 | 0.106780643 | 0.001676095 |
| Mapk13 | 0.182439638 | 0.001780192 |
| Adamts17 | 0.020533325 | 0.001806394 |
| Ccl5 | 0.15203125 | 0.001955359 |
| Psmb9 | 0.377603459 | 0.002036443 |
| Ndufa7 | 0.638262731 | 0.002038307 |
| Irf5 | 0.394786965 | 0.002041658 |
| Gtf2h2 | 3.490968016 | 0.002143876 |
| Laptm5 | 0.337861637 | 0.002159452 |
| Prdx3 | 1.746327546 | 0.002222301 |
| Tmem173 | 3.62106182 | 0.002286826 |
| Map2k3 | 0.233253009 | 0.002419614 |
| Grn | 0.397807968 | 0.002466137 |
| Nmb | 0.324944719 | 0.002506282 |
| Fem1c | 0.259284546 | 0.00252641 |
| Il1m | 0.325819717 | 0.002532944 |
| Vegfa | 0.44119421 | 0.002605017 |
| Rgl1 | 0.35368442 | 0.002613195 |
| Vsir | 0.422711736 | 0.002640791 |
| Birc2 | 2.137382912 | 0.002671959 |
| Zfp148 | 0.259527396 | 0.00268144 |
| Cd36 | 0.065889118 | 0.002728147 |
| Pdgfb | 0.267082602 | 0.002818621 |
| Mcm5 | 3.245925377 | 0.002932982 |
| Itgax | 0.475404313 | 0.002933846 |
| Vcam1 | 0.091424159 | 0.002967836 |
| Ccr3 | 0.206612829 | 0.002983667 |
| Tlr13 | 0.341624082 | 0.003000475 |
| Pparg | 0.112441722 | 0.003049328 |
| Il4i1 | 0.316034483 | 0.003127383 |
| Map3k4 | 2.384713579 | 0.003242691 |
| Sirpa | 0.264259584 | 0.003379241 |
| Il22 | 0.380217786 | 0.003421344 |
| Trem2 | 0.280912995 | 0.003426167 |
| Psme2 | 0.186946726 | 0.003492661 |
| 2810417H13Rik | 1.989489489 | 0.003528265 |
| Ikzf1 | 3.537390646 | 0.003568698 |
| C1qa | 0.353849934 | 0.003586274 |
| Itgb7 | 0.334746598 | 0.00359779 |
| Pdgfrb | 0.382473502 | 0.003620214 |
| Gpr65 | 0.367477522 | 0.003692528 |
| Cldn1 | 0.176799733 | 0.003914841 |
| Aoah | 0.185138802 | 0.003976055 |
| Fpr1 | 0.237783238 | 0.004024596 |
| Cdh5 | 0.318796992 | 0.004217463 |
| Cstb | 0.509319698 | 0.004277676 |
| Tnfaip3 | 0.362488871 | 0.004344324 |
| Tlr4 | 0.313485114 | 0.004389337 |
| Gata2 | 0.247390538 | 0.004474555 |
| Adgre5 | 0.267420622 | 0.004504331 |
| Fabp4 | 0.081952527 | 0.004615664 |
| F11r | 0.577225543 | 0.004619979 |
| Selp | 0.461080658 | 0.004649951 |
| Pbx1 | 0.056504143 | 0.004701444 |
| Pglyrp1 | 0.24386149 | 0.004731136 |
| Nod2 | 0.202589128 | 0.004754504 |
| Cytip | 3.189440036 | 0.004816784 |
| C5ar1 | 0.310848728 | 0.004932498 |
| Stat3 | 0.54619698 | 0.004978282 |
| H2-M3 | 0.544797014 | 0.005030138 |
| Fcgr4 | 0.206949182 | 0.005114836 |
| Glg1 | 0.458332786 | 0.005131973 |
| Ell2 | 2.797364709 | 0.005133794 |
| Txn1 | 2.550966829 | 0.005261062 |
| Ltb4r1 | 0.094595265 | 0.005329784 |
| Tuba4a | 7.847138456 | 0.005339111 |
| Stat5a | 0.17576126 | 0.005353731 |
| Cd84 | 0.324833922 | 0.005382937 |
| Nod1 | 0.382414393 | 0.005383592 |
| Coasy | 1.600661278 | 0.005436799 |
| Tgfb1 | 0.415703341 | 0.005467363 |
| Ptafr | 0.325647583 | 0.005547564 |
| Trex1 | 0.316274973 | 0.005633256 |
| Syk | 0.632326013 | 0.005723163 |
| Fosl1 | 0.357911207 | 0.005739431 |
| Anxa4 | 0.339804881 | 0.005813386 |
| Klk1 | 0.348398913 | 0.005860274 |
| Il18 | 0.201160428 | 0.005901136 |
| Hist2h2aa1 | 4.313462456 | 0.0060346 |
| Vamp2 | 0.159612209 | 0.006144887 |
| Rad51 | 0.321939111 | 0.0061706 |
| C1qc | 0.32866603 | 0.006253171 |
| Enpep | 0.150230112 | 0.006255852 |
| Il15 | 0.421034483 | 0.006261229 |
| Id3 | 0.384188312 | 0.006287585 |
| Fcgr1 | 0.428641232 | 0.00633297 |
| Tlr7 | 0.271087911 | 0.006458315 |
| Tek | 0.270497054 | 0.006487306 |
| Rhoj | 0.456196881 | 0.006502414 |
| H2-K1 | 0.414108849 | 0.006568241 |
| Map2k1 | 1.847722979 | 0.006655604 |
| Olr1 | 0.329073482 | 0.006725268 |
| H2-D1 | 0.281206984 | 0.007086067 |
| Lrp5 | 0.394136047 | 0.007198291 |
| Nfyc | 0.220795578 | 0.00725825 |
| Mrc1 | 0.270059412 | 0.007390174 |
| Adamts3 | 0.227611579 | 0.00767808 |
| Il1r2 | 0.345000986 | 0.007684068 |
| S1pr1 | 0.3681799 | 0.007781254 |
| Mafb | 0.404648139 | 0.008106897 |
| Il21r | 0.489325915 | 0.008347604 |
| Lpl | 0.189782517 | 0.00837903 |
| Kif20a | 4.178260304 | 0.008627483 |
| Cnn2 | 0.566633543 | 0.008998487 |
| Bcl6 | 0.293955695 | 0.009643163 |
| Csf3 | 0.142819706 | 0.009704449 |
| Id1 | 0.646807154 | 0.009712198 |
| Ncf2 | 0.402848227 | 0.01007096 |
| Flt1 | 0.168089667 | 0.010123229 |
| Ccr5 | 0.449962236 | 0.010189669 |
| Acot3 | 0.101006749 | 0.010207091 |
| Marco | 0.077207862 | 0.010261874 |
| Sell | 0.259881015 | 0.010273947 |
| Cd209e | 0.315646259 | 0.010470256 |
| Cd274 | 0.507679296 | 0.010497171 |
| Bcl2 | 0.021155496 | 0.010589704 |
| Itgb2 | 0.302117575 | 0.010859782 |
| Clec7a | 0.195973489 | 0.010926971 |
| Serpinb6a | 0.38507112 | 0.011196549 |
| Fbp1 | 0.345845289 | 0.011231073 |
| Nr1h3 | 0.086915247 | 0.011232729 |
| Cdh2 | 0.03401279 | 0.011355572 |
| Eif4ebp2 | 0.430828635 | 0.011401266 |
| Nrip3 | 0.109963926 | 0.011540563 |
| Ngf | 0.138757942 | 0.011603848 |
| Ptprc | 0.471413946 | 0.011620701 |
| Insig1 | 5.956572908 | 0.011704256 |
| Hist1h1c | 4.35048789 | 0.011736977 |
| Map2k2 | 1.600864645 | 0.011865036 |
| Irf8 | 0.383637022 | 0.012082814 |
| Cd1d1 | 1.667408377 | 0.012089349 |
| Tpsb2 | 0.28791378 | 0.012637248 |
| Map2k4 | 1.853402597 | 0.012824608 |
| Fasn | 7.41299546 | 0.013158774 |
| Top2a | 3.374740973 | 0.01352726 |
| Tap1 | 0.62158789 | 0.013630671 |
| Map3k14 | 0.324625093 | 0.013745112 |
| Rictor | 0.25476444 | 0.013862753 |
| Cxcl16 | 0.462758584 | 0.013957498 |
| Ski | 0.543395238 | 0.014210735 |
| C3ar1 | 0.291165465 | 0.014363286 |
| Yes1 | 1.768143077 | 0.014582271 |
| Il25 | 0.352201258 | 0.014655542 |
| Krba1 | 0.587368421 | 0.014716196 |
| Prok2 | 0.179305913 | 0.015130531 |
| Ifnar2 | 0.564553825 | 0.015162729 |
| Cel19 | 0.281476122 | 0.015189048 |
| Hgf | 0.047813959 | 0.015236706 |
| Tnfrsf1b | 0.465118686 | 0.015446999 |
| Cd83 | 0.343678344 | 0.015549574 |
| Gzma | 0.197484771 | 0.01567207 |
| Areg | 0.219528967 | 0.01569434 |
| Ccr9 | 0.307698229 | 0.01576045 |
| Il1rl1 | 0.594758785 | 0.016003275 |
| Ldlr | 1.893737228 | 0.016129574 |
| Dhrs3 | 0.236460574 | 0.016438206 |
| Daglb | 0.617651371 | 0.01665223 |
| TnfrsfS | 0.026620575 | 0.016760212 |
| Tlr9 | 0.452816386 | 0.017406051 |
| Yap1 | 1.767350374 | 0.017422038 |
| Il10 | 6.07902126 | 0.017469808 |
| Ptprb | 0.279432116 | 0.017502119 |
| Retnlb | 0.172507965 | 0.017551866 |
| Havcr1 | 0.218121461 | 0.017601329 |
| Cebpg | 0.568579627 | 0.017629644 |
| Itgam | 0.42803104 | 0.01778984 |
| Ctsg | 0.177480464 | 0.017922208 |
| Cttnbp2 | 0.295309654 | 0.018298116 |
| Tnfrsf1a | 0.540073297 | 0.018408459 |
| Hmgb1 | 1.353313951 | 0.018877965 |
| Arf6 | 1.211115554 | 0.018901871 |
| Csf2ra | 0.291973195 | 0.019253172 |
| Il3 | 0.147061904 | 0.019335511 |
| Msh2 | 2.548742933 | 0.019337073 |
| Igf1 | 0.354252188 | 0.019629017 |
| Met | 21.08583026 | 0.019727234 |
| Crabp2 | 2.591603335 | 0.019967264 |
| Prkci | 1.807028112 | 0.020127567 |
| Pik3cg | 2.76533915 | 0.020202275 |
| Ctss | 0.577588357 | 0.02070619 |
| Dusp1 | 0.460543084 | 0.021095308 |
| Cel12 | 0.482945654 | 0.021113635 |
| Ccl4 | 0.170247046 | 0.021276084 |
| Gadd45b | 2.190160562 | 0.021716522 |
| Cd70 | 0.095410005 | 0.021840881 |
| Id2 | 0.349878436 | 0.0219658 |
| Xcr1 | 0.16515919 | 0.021986378 |
| Serpinb2 | 0.390265997 | 0.022005334 |
| Elane | 0.219925449 | 0.022104754 |
| Icam1 | 0.410856746 | 0.022250049 |
| Stab1 | 0.435368783 | 0.022626406 |
| Myc | 8.385836329 | 0.022636534 |
| Thbd | 0.378983807 | 0.022674198 |
| Rgs16 | 0.19498524 | 0.022871446 |
| Armc1 | 1.763554589 | 0.0229913 |
| Sgpp1 | 0.603223304 | 0.023253805 |
| Fpr-rs6 | 0.134496994 | 0.023338982 |
| Irf4 | 22.0639313 | 0.023455843 |
| Ifhgr1 | 0.56146821 | 0.023615733 |
| Trem1 | 0.301053688 | 0.023628615 |
| Pdzk1ip1 | 0.164989216 | 0.02374813 |
| Ptgds | 0.122039859 | 0.023757401 |
| Tap2 | 0.555626618 | 0.0237754 |
| Cdl80 | 0.451906857 | 0.023934398 |
| Des | 0.066452579 | 0.024039289 |
| Hpgds | 0.365395309 | 0.02405374 |
| Myd88 | 0.759262602 | 0.024108207 |
| Maff | 0.370429095 | 0.024427297 |
| Tyrobp | 0.410330561 | 0.024581407 |
| Tlr3 | 0.319599363 | 0.024726185 |
| Birc3 | 1.980013984 | 0.025188469 |
| Tlr2 | 0.520085231 | 0.025376476 |
| Ptgdr2 | 0.204576043 | 0.025472165 |
| Furin | 0.616126399 | 0.0254888 |
| Tspan8 | 0.330258037 | 0.02561356 |
| Rbp1 | 0.188335046 | 0.025890911 |
| Itga4 | 0.495892195 | 0.025966941 |
| Irf7 | 0.385904357 | 0.025998641 |
| Tspan7 | 0.239566172 | 0.02618757 |
| Tlr6 | 0.321557318 | 0.026250106 |
| Ctsa | 0.235499819 | 0.026355456 |
| Adora3 | 0.174055129 | 0.026405609 |
| Smad1 | 0.556287 | 0.026733225 |
| Angpt2 | 0.136267408 | 0.026791797 |
| Znrf2 | 1.329227765 | 0.027228244 |
| Sema6d | 0.377565007 | 0.027388228 |
| Cd163 | 0.34323524 | 0.027415485 |
| Cebpa | 0.417894194 | 0.028024847 |
| Saa1 | 0.233112066 | 0.028514551 |
| Atf3 | 0.143852832 | 0.028582239 |
| Btla | 0.247276223 | 0.029007217 |
| Ccnb2 | 2.22461258 | 0.029176194 |
| Btg2 | 7.463621233 | 0.029601668 |
| Sptbn1 | 0.652949981 | 0.029695613 |
| Nectin1 | 0.537979573 | 0.029979433 |
| Rhog | 0.571960443 | 0.030106528 |
| P2rx1 | 0.213515457 | 0.030310802 |
| Cd276 | 0.672164207 | 0.030563805 |
| Sele | 0.240903388 | 0.0314953 |
| Col12a1 | 6.545076522 | 0.031594826 |
| Hivep1 | 0.507092682 | 0.032049079 |
| Tgfa | 0.397528321 | 0.032103456 |
| Il10ra | 0.468014904 | 0.032118207 |
| Mapk11 | 0.526811898 | 0.032123992 |
| Ptgs2 | 2.465932359 | 0.032231081 |
| Mif | 1.622067876 | 0.032233171 |
| Adam8 | 0.071527939 | 0.032416701 |
| Amica1 | 2.718984784 | 0.032428194 |
| Kalrn | 0.574808592 | 0.032479516 |
| Lif | 0.357903956 | 0.033138142 |
| Cyp3a13 | 0.118017502 | 0.033589365 |
| Ccl6 | 0.321187767 | 0.033820377 |
| Cel11 | 0.41302283 | 0.034194143 |
| Pdcd1lg2 | 0.203772944 | 0.03449647 |
| Ifnb1 | 0.152625468 | 0.034569885 |
| Rasgrf2 | 0.241724618 | 0.034678222 |
| Ms4a2 | 0.324938575 | 0.034913436 |
| Nr4a1 | 0.362822165 | 0.035198737 |
| Nr4a2 | 0.1847315 | 0.035325177 |
| Pim2 | 1.525437443 | 0.035597185 |
| Daxx | 0.171630869 | 0.036261736 |
| Ccl8 | 0.356093831 | 0.036524392 |
| Kitl | 0.408832042 | 0.036561693 |
| Vav2 | 0.598886165 | 0.036764165 |
| Selplg | 0.585317401 | 0.036785756 |
| Cd44 | 0.519344484 | 0.036880806 |
| Tnc | 6.053200875 | 0.037108506 |
| Cdh13 | 0.298271068 | 0.037266519 |
| Ttk | 2.124135506 | 0.039002007 |
| Egf | 0.241775255 | 0.03934799 |
| Cxcl3 | 0.39950602 | 0.039399474 |
| Ifit1bl1 | 0.341780881 | 0.039850713 |
| Ccrl2 | 0.27009876 | 0.040213652 |
| Cd47 | 0.658483034 | 0.041716587 |
| Myod1 | 0.137666606 | 0.041835817 |
| Lst1 | 0.365902999 | 0.042076443 |
| Ets1 | 0.881746058 | 0.042265839 |
| Cma1 | 0.321321933 | 0.042740779 |
| Il3ra | 0.435600628 | 0.04355236 |
| Stat1 | 0.471554625 | 0.04359586 |
| Rgs6 | 0.17284502 | 0.043663574 |
| Adamts2 | 0.586995144 | 0.043792777 |
| Rapgef4 | 0.683411075 | 0.044581577 |
| Rin2 | 0.544593482 | 0.044588088 |
| Hras | 1.324562001 | 0.044739 |
| Stat4 | 0.150512894 | 0.046168799 |
| Stat6 | 0.560074537 | 0.046309173 |
| Rab3d | 1.521794262 | 0.046704299 |
| Ccr8 | 0.359370176 | 0.046873457 |
| Cxcr1 | 0.310431154 | 0.046966693 |
| Ctsl | 0.450591372 | 0.047136266 |
| Klf10 | 0.394083087 | 0.047373812 |
| Vasp | 0.68357939 | 0.048060912 |
| Acot11 | 0.38633014 | 0.048112195 |
| Il13 | 0.08717548 | 0.048566168 |
| Ptpn14 | 0.16606049 | 0.048673805 |
| Rgs1 | 0.175615144 | 0.049327452 |
| Gsn | 0.541499708 | 0.050104449 |
| Clic4 | 0.819396641 | 0.05027438 |
| Cysltr1 | 0.422849692 | 0.05049217 |
| Ccl9 | 0.388811 | 0.050764953 |

TABLE 17
Genes Differentially Expressed in FaDu immune infiltrates
vs Raji immune infiltrates at baseline

| | FaDu control | |
|---|---|---|
| | IgG/Raji control | |
| Gene Name | IgG | p-value |
| Aoah | 0.207183682 | 6.79986E−06 |
| Tnc | 10.73551299 | 1.29322E−05 |
| Sema6d | 3.43072982 | 2.72471E−05 |
| Adam19 | 4.975011478 | 0.000125526 |
| Arg1 | 0.138768338 | 0.000333034 |
| Vegfa | 2.678260117 | 0.000391212 |
| Ccr7 | 0.272783849 | 0.000418332 |
| Isg15 | 4.089362311 | 0.000460231 |
| Cd80 | 0.332909508 | 0.000464953 |
| Cytip | 0.375667039 | 0.000594026 |
| Il4i1 | 0.14447383 | 0.000620695 |
| Flt1 | 3.599589924 | 0.000651081 |
| Il1rap | 4.405514339 | 0.000654972 |
| Kalrn | 2.95481528 | 0.00071457 |
| Id1 | 2.630817951 | 0.000853238 |
| Ccl22 | 0.368778714 | 0.00089311 |
| Fcgr2b | 0.356576361 | 0.000956872 |
| Cstb | 0.394749546 | 0.001096934 |
| Tnfrsf12a | 2.405878732 | 0.00113115 |
| Mrc2 | 3.248376493 | 0.001197253 |
| Plau | 5.904855575 | 0.001354886 |
| Ptafr | 0.391455309 | 0.001389921 |
| Id2 | 0.267535304 | 0.001509436 |
| Fzd4 | 3.77913201 | 0.001593172 |
| Il1rn | 0.160803028 | 0.001656893 |
| Marcksl1 | 0.307370799 | 0.001727676 |
| Stat5a | 0.738232995 | 0.001907809 |
| Ms4a1 | 0.012789329 | 0.001963532 |
| Rnd3 | 6.829027376 | 0.001975847 |
| Pdgfrb | 3.309003992 | 0.002048025 |
| Cxcr4 | 0.542108167 | 0.002065703 |
| F11r | 1.698956329 | 0.002139103 |
| Rin2 | 1.842082006 | 0.002221817 |
| H2-T23 | 1.865162037 | 0.002287457 |
| Tgfb1 | 2.059437863 | 0.002456686 |
| Hgf | 2.542206308 | 0.002646192 |
| Fgfr1 | 2.652502767 | 0.00282469 |
| Malt1 | 0.339121675 | 0.002843367 |
| Ifit1bl1 | 2.428613876 | 0.002874609 |
| Birc2 | 0.237908208 | 0.002959863 |
| Itgam | 0.413518008 | 0.002987492 |
| Trem2 | 3.099420849 | 0.003196439 |
| Cd38 | 0.329641685 | 0.003388464 |
| Tnfrsf14 | 0.507638703 | 0.003436122 |
| Tlr3 | 2.707911018 | 0.003672377 |
| Stab1 | 2.496617369 | 0.003815057 |
| Flt3 | 0.434883444 | 0.003905285 |
| Fyn | 0.349162876 | 0.003991225 |
| 2900026A02Rik | 3.239398774 | 0.004466434 |
| Cd274 | 0.32069915 | 0.004555681 |
| Marco | 0.057835232 | 0.004564954 |
| Mapk13 | 10.6492437 | 0.005180174 |
| Mmp9 | 9.173438481 | 0.005502216 |
| Mafb | 0.542923081 | 0.005675117 |
| Adamts14 | 1.575693958 | 0.005743684 |
| Sema5a | 5.398296593 | 0.005745395 |
| Slc16a6 | 0.202711451 | 0.005800174 |
| Nectin2 | 3.850148315 | 0.005810357 |
| Cdh5 | 2.416883633 | 0.005819504 |
| Acod1 | 0.622604832 | 0.006356735 |
| Havcr2 | 2.054267681 | 0.006433338 |
| Cdh2 | 2.984106598 | 0.006715323 |
| Cxcl10 | 2.296403689 | 0.006847609 |
| Hpgds | 2.277701649 | 0.00697401 |
| Batf | 0.289324453 | 0.007805388 |
| Icosl | 1.905694908 | 0.008204495 |
| Angpt2 | 1.862653208 | 0.008209137 |
| Peli1 | 0.177153654 | 0.008544832 |
| Hif1a | 0.564790337 | 0.008614756 |
| Ccl20 | 6.061919505 | 0.008683558 |
| Ptgs1 | 5.949399815 | 0.008738777 |
| Itgb1 | 1.699566297 | 0.008841093 |
| Timp3 | 3.824227156 | 0.009022777 |
| Adamts4 | 5.350343107 | 0.009222181 |
| Cd14 | 0.374516108 | 0.009242469 |
| Col4a2 | 2.054584548 | 0.009286182 |
| Cyr61 | 3.206349206 | 0.009399745 |
| Rhoc | 3.465748709 | 0.009643269 |
| Lrp5 | 1.958864948 | 0.009722287 |
| Cxcl14 | 4.232474296 | 0.009723622 |
| Ctgf | 9.417441293 | 0.009920781 |
| Amica1 | 0.362901842 | 0.009949818 |
| Cxcl13 | 0.012018189 | 0.010452425 |
| Cd276 | 1.727977311 | 0.010456879 |
| S1pr1 | 1.607531422 | 0.010634661 |
| Ptprk | 1.723240281 | 0.01160142 |
| Skil | 1.249656325 | 0.011697016 |
| Cdh13 | 1.6689524 | 0.011768746 |
| Col12a1 | 12.09120681 | 0.011843407 |
| Irf7 | 2.636049316 | 0.011870486 |
| Jun | 7.668374405 | 0.01195414 |
| Nr4a2 | 6.958977408 | 0.01200905 |
| Col11a1 | 7.124295775 | 0.012012393 |
| Cd69 | 2.16439471 | 0.012141834 |
| Lrp6 | 6.146120613 | 0.012508717 |
| Il1b | 0.407198064 | 0.012973786 |
| Yes1 | 12.15297593 | 0.013030479 |
| Dusp6 | 2.161453237 | 0.013475858 |
| Bcl2 | 0.126375006 | 0.013555608 |
| Trafd1 | 1.793987205 | 0.013571667 |
| Gadd45b | 0.46110908 | 0.013581999 |
| Loxl2 | 3.59653792 | 0.013769808 |
| Rapgef4 | 4.613133372 | 0.013771525 |
| Ccrl2 | 0.515928144 | 0.014343021 |
| Birc5 | 2.319407407 | 0.014592366 |
| Casp7 | 1.837298542 | 0.014963521 |
| Tnfaip8 | 0.689326065 | 0.015571626 |
| Egr3 | 1.884713584 | 0.015589093 |
| Nr2f6 | 1.43321049 | 0.015705965 |
| Phlda2 | 4.613061354 | 0.016022829 |
| Cdkn1a | 0.676097325 | 0.016312913 |
| Col4a1 | 2.397402915 | 0.016852976 |
| Ccr2 | 0.430079253 | 0.016942951 |
| Tep1 | 1.660609827 | 0.016979693 |
| Cnn2 | 0.399559673 | 0.01716429 |
| B4galt4 | 2.526235809 | 0.017708886 |
| Traf1 | 0.145346741 | 0.017827922 |
| Enpp2 | 0.101856734 | 0.018089745 |
| Rgl1 | 0.630572947 | 0.018127793 |
| Adamts12 | 6.319657439 | 0.018681404 |
| Ski | 1.631040307 | 0.018709768 |
| Gnai3 | 1.538667504 | 0.018880092 |
| Furin | 3.844485149 | 0.019045269 |
| Sema4a | 0.352081071 | 0.019419019 |
| Itga5 | 2.928712799 | 0.01969598 |
| Smad1 | 1.334790847 | 0.019971945 |
| Icam1 | 0.321822966 | 0.020093983 |
| Man2b1 | 2.578551833 | 0.021161168 |
| Hoxd4 | 1.436039768 | 0.021178693 |
| Fas | 0.424483631 | 0.021326811 |
| C1qc | 1.407277699 | 0.021565863 |
| Col1a2 | 6.661792912 | 0.021649114 |
| Cdh11 | 4.476532144 | 0.021894145 |
| Smad7 | 1.335587164 | 0.022095249 |
| Btg2 | 0.633851298 | 0.022123606 |
| Ifngr1 | 0.598779292 | 0.022260197 |
| Ptprb | 2.254560685 | 0.022433857 |
| Flnb | 1.724331726 | 0.022734687 |
| Rgs16 | 2.55458497 | 0.022736377 |
| C1qa | 1.501307958 | 0.02278472 |
| C1qb | 1.480747189 | 0.023183528 |
| C5ar1 | 0.545580987 | 0.023482547 |
| Klk1 | 0.705083675 | 0.023700012 |
| Vav1 | 0.636284105 | 0.024324405 |
| Apoe | 3.495786081 | 0.024540518 |
| Dusp1 | 0.362124967 | 0.024859227 |
| Grn | 1.45908889 | 0.025546804 |
| Arhgef28 | 2.056808231 | 0.026377291 |
| Nfil3 | 0.419020827 | 0.026542819 |
| Nr1h3 | 0.423280037 | 0.026616761 |
| Hist2h2aa1 | 1.978653237 | 0.026743248 |
| Nfkbie | 0.261582273 | 0.027143655 |
| Adamts17 | 5.827548483 | 0.027524808 |
| Met | 5.620529738 | 0.029020644 |
| Lipa | 1.751483372 | 0.02902514 |
| Mmp13 | 55.55189898 | 0.029394538 |
| Sirpa | 0.423486083 | 0.029485859 |
| Tlr2 | 0.383417153 | 0.02949126 |
| Fpr-rs4 | 0.305222631 | 0.029756941 |
| Usp18 | 3.204732824 | 0.030941516 |
| Irf5 | 1.763903463 | 0.031021183 |
| Ets1 | 1.389771526 | 0.03212633 |
| Mapk14 | 1.470464446 | 0.032803894 |
| Stat3 | 1.380297848 | 0.032874938 |
| Insr | 2.059068535 | 0.033597223 |
| Ifnar1 | 0.685137511 | 0.033677557 |
| Glg1 | 1.387811359 | 0.033977826 |
| Birc3 | 0.427430662 | 0.035233813 |
| Klrk1 | 1.976549094 | 0.035256523 |
| Retnla | 2.921773612 | 0.035277702 |
| I13ra | 1.583552403 | 0.035376054 |
| Fpr-rs5 | 0.133303955 | 0.03551874 |
| Pf4 | 0.303808232 | 0.035606801 |
| Klf4 | 2.018783037 | 0.036396788 |
| C3ar1 | 0.781383741 | 0.036563244 |
| Smarcd3 | 0.357327629 | 0.036655795 |
| Sele | 0.371328042 | 0.037026044 |
| Ccr1 | 0.664316116 | 0.038047937 |
| Ell2 | 2.086860375 | 0.038922857 |
| Cx3cl1 | 1.884932799 | 0.039421471 |
| Ndufa7 | 0.816020139 | 0.039557856 |
| Cdc20 | 2.474682412 | 0.039953566 |
| Acot3 | 0.453058419 | 0.04007237 |
| Kif20a | 1.543991695 | 0.041191621 |
| Cxcl16 | 0.627112916 | 0.041294948 |
| Id3 | 4.285029191 | 0.041370933 |
| Mmp10 | 2.718676123 | 0.042058231 |
| Lamb2 | 3.563406255 | 0.043090642 |
| Cd84 | 0.738356021 | 0.043213422 |
| Mx2 | 3.56378071 | 0.044221387 |
| Pdgfa | 1.830474098 | 0.04469437 |
| En1 | 2.584862385 | 0.044754893 |
| Fut4 | 1.544391785 | 0.044941179 |
| Fasn | 1.370010829 | 0.046002222 |
| Akap13 | 2.242516762 | 0.046924678 |
| Tmem173 | 0.785006231 | 0.04713767 |
| Golim4 | 1.862494168 | 0.048385854 |
| Hspg2 | 2.125878136 | 0.048547954 |
| Tlr7 | 2.273414179 | 0.048864907 |
| Ctnnb1 | 1.794792155 | 0.049322421 |
| Prrx1 | 2.772447125 | 0.049704088 |
| Kcnab1 | 2.038793103 | 0.050582403 |
| Ndc80 | 1.714388749 | 0.050905658 |

TABLE 18
Genes Differentially Expressed in FaDu immune infiltrates
vs RPMI-8226 immune infiltrates at baseline

| | FaDu control IgG over | |
|---|---|---|
| Gene Name | RPMI-8226 control IgG | p-value |
| Peli1 | 0.18768212 | 1.61133E−05 |
| Adam1 9 | 3.572626225 | 9.58506E−05 |
| Adamts17 | 0.236891257 | 0.000243599 |
| Il1rap | 5.278333333 | 0.000304276 |
| Vegfa | 1.304191473 | 0.000308512 |
| Tnfrsf12a | 2.810448351 | 0.000424505 |
| F11r | 2.253101954 | 0.000476521 |
| Tnc | 10.50625161 | 0.000477441 |
| Plau | 2.240092691 | 0.000524193 |
| Sirpa | 0.657230973 | 0.000705723 |
| Tnfaip8 | 0.370308124 | 0.000746355 |
| Cxcl12 | 0.157191709 | 0.000803011 |
| Lipa | 0.662834677 | 0.000833945 |
| Skil | 1.857427749 | 0.0008541 |
| Fut4 | 3.098246355 | 0.000976421 |
| Smad7 | 0.29035371 | 0.001037132 |
| Pbx1 | 0.144135256 | 0.001150892 |
| Id1 | 1.677792246 | 0.001298079 |
| Insig1 | 2.51930093 | 0.001311256 |
| Kalrn | 2.249574649 | 0.001387444 |
| Cxcl2 | 4.076089695 | 0.001495592 |
| Fscn1 | 1.927468983 | 0.001539672 |
| Ctsd | 0.54830471 | 0.001798022 |
| Rnd3 | 1.969129469 | 0.001815788 |
| Slc16a6 | 0.483729984 | 0.002141001 |
| Csf1r | 1.718517008 | 0.002236647 |
| Mmp10 | 7.095396298 | 0.002469028 |
| S1pr1 | 1.950804847 | 0.002484998 |
| Cd40 | 2.081276625 | 0.00253892 |
| Tlr1 | 1.774788494 | 0.002952808 |
| Vcam1 | 0.364311675 | 0.003374235 |
| Mrc2 | 1.920692107 | 0.003377614 |
| Gnai3 | 1.918488192 | 0.003415686 |
| Tgfb1 | 1.500128594 | 0.003760311 |
| Pik3cg | 1.769701661 | 0.003900229 |
| Fzd4 | 1.671279773 | 0.003977657 |
| Tnfaip3 | 6.563783303 | 0.004406902 |
| Col15a1 | 0.499057071 | 0.004621974 |
| Ccr3 | 0.432424196 | 0.004733328 |
| Smad1 | 1.518020365 | 0.004795512 |
| Mmp9 | 46.63021029 | 0.004912208 |
| Cd69 | 3.319188768 | 0.005050295 |
| Tmem173 | 2.199587334 | 0.00525771 |
| Fgfr1 | 3.134241599 | 0.005465508 |
| Cyr61 | 3.355968773 | 0.005539533 |
| Fasn | 2.173456866 | 0.006003921 |
| Stat5a | 0.22077549 | 0.006173539 |
| Fcgr4 | 0.493562887 | 0.00659735 |
| Mertk | 0.602507428 | 0.006931611 |
| Itgal | 3.718439959 | 0.007264248 |
| Cd68 | 0.479950298 | 0.008401722 |
| Plaur | 2.816503412 | 0.008613263 |
| Loxl2 | 2.493789253 | 0.008854302 |
| Isg15 | 2.200500096 | 0.008932882 |
| Col12a1 | 8.632185411 | 0.008990743 |
| Ctgf | 2.574726669 | 0.009017569 |
| Fbp1 | 0.389705452 | 0.009303977 |
| Cxcl14 | 8.551041329 | 0.009623193 |
| Timp3 | 2.280532081 | 0.009963742 |
| Irf5 | 1.359642028 | 0.010074978 |
| Havcr2 | 2.063762963 | 0.010293637 |
| Fyn | 1.654512285 | 0.010352584 |
| Tapbp | 1.835812199 | 0.010538308 |
| Flnb | 3.022679429 | 0.010648043 |
| Rhoc | 2.60747954 | 0.010797781 |
| Ccr7 | 1.918758852 | 0.011482435 |
| Pggt1b | 0.474407973 | 0.01152401 |
| Bcl2 | 0.079937909 | 0.011567927 |
| Acot3 | 0.364642106 | 0.01197054 |
| 2900026A02Rik | 2.106360906 | 0.012744048 |
| Cdh2 | 0.108751997 | 0.013168635 |
| Casp7 | 1.702837272 | 0.013274046 |
| Osm | 2.887572371 | 0.013519473 |
| Nectin2 | 2.99919265 | 0.013544018 |
| Birc5 | 1.883995187 | 0.013637446 |
| Vasp | 1.774282962 | 0.014633644 |
| Lif | 2.049043935 | 0.01491724 |
| Adamts14 | 2.262602579 | 0.015071512 |
| Enpep | 0.417735043 | 0.015265656 |
| Marcksl1 | 2.524752144 | 0.015609061 |
| Yes1 | 6.093977924 | 0.015856047 |
| Tnfrsf8 | 0.062703439 | 0.016027542 |
| Stab1 | 1.706963319 | 0.016134768 |
| Col4a2 | 0.745889705 | 0.016645965 |
| Cdh5 | 1.384191571 | 0.017124817 |
| Egr3 | 3.042269893 | 0.017162442 |
| Hgf | 0.102849481 | 0.017201842 |
| Ptprk | 1.778995757 | 0.017947776 |
| Dusp6 | 1.975421541 | 0.018183101 |
| Cdh4 | 0.303100551 | 0.018337074 |
| Map2k1 | 2.027472969 | 0.019009229 |
| Rapgef4 | 3.789379261 | 0.019222846 |
| Aoah | 0.438843665 | 0.019422086 |
| Nfyc | 0.657709516 | 0.019593883 |
| Klf4 | 3.73648245 | 0.020011509 |
| Adamts4 | 2.424069416 | 0.020259593 |
| Itga5 | 2.866001937 | 0.0204955 |
| Ccr1 | 3.229865404 | 0.02092543 |
| C1qc | 0.776336111 | 0.021273327 |
| Tlr8 | 0.709680939 | 0.021574505 |
| Cebpg | 1.33902439 | 0.021806488 |
| Col11a1 | 4.200332157 | 0.021927739 |
| Dusp2 | 2.596335697 | 0.022259791 |
| Fem1c | 0.664409187 | 0.022422421 |
| Gpr183 | 1.804557406 | 0.02254009 |
| Maff | 1.649291448 | 0.022977956 |
| Traf2 | 1.461970435 | 0.024113066 |
| Met | 34.70376384 | 0.025504301 |
| Tlr9 | 1.325091441 | 0.026500434 |
| H2-Aa | 2.641495069 | 0.026649405 |
| Nfkbia | 3.390448129 | 0.026715846 |
| Adamts9 | 1.585083848 | 0.026957329 |
| Il1b | 10.28773876 | 0.027129949 |
| Col1a2 | 3.950084341 | 0.027295803 |
| Hif1a | 2.565578772 | 0.028373179 |
| Nlrp3 | 2.654542733 | 0.028825404 |
| Tyrobp | 1.15497664 | 0.02930473 |
| Ccrl2 | 1.719941164 | 0.029458701 |
| Map2k2 | 1.710439316 | 0.029531561 |
| Furin | 2.080262749 | 0.029569393 |
| Cdh11 | 4.032277778 | 0.029602114 |
| Id2 | 0.437326701 | 0.029953832 |
| Adamts12 | 2.160580131 | 0.030391671 |
| Mmp13 | 28.20497377 | 0.030407624 |
| Ier3 | 2.804285041 | 0.030890274 |
| Ptprc | 1.23115192 | 0.031208075 |
| Cdh1 | 1.849677111 | 0.031480612 |
| Smarcd3 | 2.466477809 | 0.031489787 |
| Edn1 | 2.079403606 | 0.031783443 |
| B4galt4 | 2.002479983 | 0.032971173 |
| Cxcl10 | 2.408990093 | 0.032974617 |
| Stat1 | 1.579187522 | 0.033101451 |
| Enc1 | 2.396871539 | 0.033297796 |
| Cd74 | 3.078835669 | 0.034368301 |
| Ctsg | 0.391901492 | 0.03441609 |
| Tuba1a | 1.811046493 | 0.03567023 |
| H2-DMa | 2.164545962 | 0.03567165 |
| Man2b1 | 1.916689324 | 0.035851914 |
| Myd88 | 0.791749388 | 0.035949136 |
| Ccl20 | 2.469104666 | 0.036088668 |
| Tlr3 | 1.560968207 | 0.036101859 |
| Tnfrsf14 | 1.571539698 | 0.036638999 |
| Usp18 | 2.069710116 | 0.037802141 |
| Il6 | 4.657358156 | 0.038173508 |
| C1qb | 0.768709016 | 0.038225725 |
| Ndufa7 | 0.833149348 | 0.038776774 |
| Ifnar1 | 1.89015105 | 0.039030543 |
| Ctsa | 0.427227782 | 0.039654132 |
| Icosl | 1.92926816 | 0.039840874 |
| Marco | 0.554725363 | 0.040245019 |
| Elane | 0.413080312 | 0.04129714 |
| Ctnnb1 | 2.335714231 | 0.041397502 |
| Cx3cl1 | 1.587370929 | 0.041459315 |
| Tlr2 | 1.395452005 | 0.042351921 |
| Hspg2 | 2.062409158 | 0.042987507 |
| Cxcr2 | 12.08270677 | 0.043558074 |
| Psmb9 | 0.646201213 | 0.043662584 |
| Bel10 | 1.524742268 | 0.045507322 |
| Serpine1 | 2.171619028 | 0.046609236 |
| Sphk1 | 2.494498778 | 0.046913105 |
| Anxa4 | 0.651370282 | 0.047548291 |
| Stat3 | 1.273780994 | 0.048197661 |
| Cd276 | 1.411177445 | 0.048403223 |
| Csf3r | 3.328641435 | 0.049023183 |
| Socs3 | 1.381235892 | 0.049029875 |
| H2-Ab1 | 3.012979396 | 0.050198212 |
| Nfkbiz | 1.927282478 | 0.050308599 |
| Jun | 1.707738167 | 0.050773742 |
| Il18r1 | 0.473691032 | 0.050981292 |

Of the differentially expressed human genes in non-responders versus responders, 73 genes were enriched in both non-responder cell lines as shown in Table 19 and 86 genes were enriched in both responder tumors and as shown in Table 19.

TABLE 19
Common genes differentially expressed at baseline
in Non-Responder tumors vs Responder tumors

| Unregulated in Non- | Unregulated in |
|---|---|
| Responder Compared | Responder Compared |
| to Responder | to Non-Responder |
| ANGPTL4 | ADORA2A |
| ANLN | ARG2 |
| APH1B | ARID1A |
| AREG | ATM |
| AXL | BCL2 |
| BMP2 | BIRC3 |
| CBLC | BLK |
| CCL20 | CASP9 |
| CCNA1 | CCL3/L1 |
| CCND1 | CD38 |
| CD276 | CD40 |
| CD44 | CD45RA |
| CDH1 | CD45RB |
| CEBPB | CD45RO |
| COL17A1 | CD48 |
| CRABP2 | CD70 |
| CX3CL1 | CD74 |
| CXCL1 | CD79A |
| CXCL2 | CD79B |
| CXCL3 | CD84 |
| CXCL8 | CD86 |
| DKK1 | CSF2RB |
| EDN1 | CTSS |
| EGFR | CXCL10 |
| ERBB2 | CXCL9 |
| F2RL1 | CXCR4 |
| FADD | CYBB |
| FOSL1 | DUSP2 |
| FSTL3 | E2F3 |
| GPC4 | ENTPD1 |
| HERC6 | FCGR2A |
| HES1 | FCGR2B |
| IER3 | FCRL2 |
| IFI27 | GPR160 |
| IFIT2 | GPSM3 |
| IFITM2 | HAVCR2 |
| IL11 | HLA-A |
| IL18 | HLA-DMA |
| IL1A | HLA-DMB |
| IL1B | HLA-DOB |
| IL6 | HLA-DPA1 |
| ITGA2 | HLA-DPB1 |
| ITGA6 | HLA-DRA |
| ITGB8 | HLA-DRB1 |
| JAG1 | HLA-F |
| JAG2 | ICAM2 |
| LAMB3 | ICAM3 |
| LAMC2 | ICAM5 |
| LIF | ICOSLG |
| MET | IL10 |
| MMP1 | IL10RA |
| NECTIN1 | IL16 |
| NECTIN2 | IL21R |
| NOTCH1 | IL2RG |
| OAS3 | IRF4 |
| OASL | IRF8 |
| P4HA2 | ITGA4 |
| PC | ITGAL |
| PLOD2 | KAT2B |
| PPARG | LY9 |
| PVR | MFNG |
| RNLS | MS4A1 |
| S100A9 | NFATC2 |
| SAMD9 | PECAM1 |
| SERPINB5 | PIK3CD |
| SERPINH1 | PIK3R1 |
| SOX2 | PNOC |
| STC1 | PPARGC1B |
| THBS1 | PTPRC |
| TNFRSF1A | RUNX3 |
| TNFSF12 | SAMSN1 |
| ULBP2 | SLAMF7 |
| WNT7B | SLC1A5 |
| | SMAP1 |
| | SYK |
| | TLR1 |
| | TLR7 |
| | TLR9 |
| | TMEM140 |
| | TNFRSF14 |
| | TNFRSF17 |
| | TNFRSF1B |
| | TNFSF4 |
| | TRAF1 |
| | WNT5A |
| | ZEB1 |

Of the differentially expressed murine genes in non-responders versus responders, four genes were enriched in both non-responder cell lines as shown in Table 20. Additionally, ten genes were enriched in both responder lines as shown in Table 20.

TABLE 20
Common genes differentially-expressed at baseline in non-responder
tumor immune infiltrates vs responder tumor immune infiltrates.

| Up | Down |
|---|---|
| Met | Ndufa7 |
| Tnc | Sirpa |
| Col12a1 | Marco |
| Yes1 | Aoah |
| | Id2 |
| | Tnfaip8 |
| | Acot3 |
| | Stat5a |
| | Peli1 |
| | Bcl2 |

The detailed description set-forth above is provided to aid those skilled in the art in practicing the present disclosure. However, the disclosure described and claimed herein is not to be limited in scope by the specific embodiments herein disclosed because these embodiments are intended as illustration of several aspects of the disclosure. Any equivalent embodiments are intended to be within the scope of this disclosure. Indeed, various modifications of the disclosure in addition to those shown and described herein will become apparent to those skilled in the art from the foregoing description, which do not depart from the spirit or scope of the present inventive discovery. Such modifications are also intended to fall within the scope of the appended claims.

Also provided are embodiments wherein any embodiment above can be combined with any one or more of these embodiments, provided the combination is not mutually exclusive. Also provided herein are uses in the treatment of indications or one or more symptoms thereof as disclosed herein and uses in the manufacture of medicaments for the treatment of indications or one or more symptoms thereof as disclosed herein, equivalent in scope to any embodiment disclosed above, or any combination thereof that is not mutually exclusive.

1. A method of treating cancer in a patient by quantifying at least one biomarker having an expression level greater or less than the amount of a baseline standard comprising: administering an effective amount of an anti-CD47 antibody to the patient, wherein the at least one biomarker is selected from Table 1 through Table 20.

2. A method of treating a cancer in a patient with an anti-CD47 antibody wherein the method comprises:
a. determining expression level of at least one biomarker chosen from the biomarkers listed in Table 1 through Table 20;
b. quantifying the level of expression of at least one biomarker indicating sensitivity to treatment,
c. estimating the susceptibility of a patient either prior to or after treatment; and
d. administering a therapeutically effective amount of an anti-CD47 antibody to the patient.

3. A method of treating cancer in a patient by quantifying at least one biomarker listed in Table 1 through Table 20 having an expression level greater or less than the amount of a baseline standard to identify patients likely to respond to treatment with an anti-CD47 treatment in combination with one or more anti-cancer therapeutic agents, wherein the one or more anti-cancer therapeutic agents is selected from an anti-cancer therapeutic agent developed against any one of the biomarkers listed in Table 1 through Table 20.

4. A method of treating cancer in a patient by quantifying at least one biomarker listed in Table 1 through Table 20 having an expression level greater or less than the amount of a baseline standard to identify one or more anti-cancer therapeutic agents which will increase therapeutic outcome in combination with an anti-CD47 antibody.

5. A method of treating cancer in a patient by quantifying at least one biomarker listed in Table 1 through Table 20 having an expression level greater or less than the amount of a baseline standard, wherein the expression level of at least one biomarker results in the development of a therapeutic agent, wherein the therapeutic agent will convert a non-responder patient population to a responder patient population.

6. A method of treating cancer in a patient by quantifying at least one biomarker listed in Table 1 through Table 20 having an expression level greater or less than the amount of a baseline standard, wherein the expression level of at least one biomarker results in the combination of an anti-CD47 antibody with a second anti-cancer therapeutic agent, wherein the combination treatment convert a non-responder patient population to a responder patient population.

7. The method of claims 1-6, wherein the quantifying is performed by measuring the amount of the biomarker in each sample by one or more methods selected from NanoString sequencing, RNAseq, qPCR, and microarray.

8. The method of claim 1-6, wherein the cancer is a solid tumor.

9. The method of claim 8, wherein the solid tumor is selected from cervical cancer, pancreatic cancer, ovarian cancer, mesothelioma, squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulval cancer, thyroid cancer, hepatic carcinoma, anal carcinoma, penile carcinoma head and neck cancer, sarcoma, adrenocortical carcinoma, adult (primary) hepatocellular cancer, adult (primary) liver cancer, or any combination thereof.

10. The method of claims 1-6, wherein the cancer is a hematological malignancy.

11. The method of claim 10 wherein the hematologic malignancy is multiple myeloma, acute childhood lymphoblastic leukemia, acute lymphoblastic l leukemia, acute lymphocytic leukemia, acute myeloid leukemia, adult acute lymphocytic leukemia, adult acute myeloid leukemia, adult Hodgkin's disease, adult Hodgkin's lymphoma, adult lymphocytic leukemia, adult Non-Hodgkin's lymphoma, AIDS-related lymphoma, or any combination thereof.

12. The method of claims 1-6, wherein the biological sample is a core biopsy, free needle aspirate, pleural effusion, resection, ascites, whole blood, blood serum, plasma, bone marrow, or other bodily fluid, or dilution thereof.

13. The method of claims 1-6, wherein at least two biomarkers are selected from Table 1-Table 20.

14. The method of claims 1-6, wherein at least three biomarkers are selected from Table 1-Table 20.

15. The method of claims 1-6, wherein at least four biomarkers are selected from Table 1-Table 20.

16. The method of claims 1-6, wherein at least five biomarkers are selected from Table 1-Table 20.

17. The method of claims 1-6, wherein at greater that five biomarkers are selected from Table 1-Table 20.

18. The method of claims 1-6, wherein the anti-CD47 antibody directly causes autonomous tumor cell death.

19. The method of claims 1-6, wherein the anti-CD47 antibody is selected from a combination of a heavy chain (HC) and a light chain (LC), wherein the combination is selected from:
(i) a heavy chain comprising the amino acid sequence of SEQ ID NO:76 and a light chain comprising the amino acid sequence SEQ ID NO:66;
(ii) a heavy chain comprising the amino acid sequence of SEQ ID NO:77 and a light chain comprising the amino acid sequence SEQ ID NO:68;
(iii) a heavy chain comprising the amino acid sequence of SEQ ID NO:78 and a light chain comprising the amino acid sequence SEQ ID NO:69;
(iv) a heavy chain comprising the amino acid sequence of SEQ ID NO:79 and a light chain comprising the amino acid sequence SEQ ID NO:70;
(v) a heavy chain comprising the amino acid sequence of SEQ ID NO:80 and a light chain comprising the amino acid sequence SEQ ID NO:70;
(vi) a heavy chain comprising the amino acid sequence of SEQ ID NO:81 and a light chain comprising the amino acid sequence SEQ ID NO:70;
(vii) a heavy chain comprising the amino acid sequence of SEQ ID NO: 82 and a light chain comprising the amino acid sequence SEQ ID NO:68;
(viii) a heavy chain comprising the amino acid sequence of SEQ ID NO: 83 and a light chain comprising the amino acid sequence SEQ ID NO:70;
(ix) a heavy chain comprising the amino acid sequence of SEQ ID NO: 84 and a light chain comprising the amino acid sequence SEQ ID NO:71;
(x) a heavy chain comprising the amino acid sequence of SEQ ID NO: 85 and a light chain comprising the amino acid sequence SEQ ID NO:72;
(xi) a heavy chain comprising the amino acid sequence of SEQ ID NO: 86 and a light chain comprising the amino acid sequence SEQ ID NO:72;
(xii) a heavy chain comprising the amino acid sequence of SEQ ID NO: 80 and a light chain comprising the amino acid sequence SEQ ID NO:73;
(xiii) a heavy chain comprising the amino acid sequence of SEQ ID NO:81 and a light chain comprising the amino acid sequence SEQ ID NO:73;
(xiv) a heavy chain comprising the amino acid sequence of SEQ ID NO: 87 and a light chain comprising the amino acid sequence SEQ ID NO:70;
(xv) a heavy chain comprising the amino acid sequence of SEQ ID NO:79 and a light chain comprising the amino acid sequence SEQ ID NO:73;
(xvi) a heavy chain comprising the amino acid sequence of SEQ ID NO: 88 and a light chain comprising the amino acid sequence SEQ ID NO:74;
(xvii) a heavy chain comprising the amino acid sequence of SEQ ID NO: 89 and a light chain comprising the amino acid sequence SEQ ID NO:74;
(xviii) a heavy chain comprising the amino acid sequence of SEQ ID NO:90 and a light chain comprising the amino acid sequence SEQ ID NO:74;
(xix) a heavy chain comprising the amino acid sequence of SEQ ID NO:91 and a light chain comprising the amino acid sequence SEQ ID NO:74;
(xx) a heavy chain comprising the amino acid sequence of SEQ ID NO: 84 and a light chain comprising the amino acid sequence SEQ ID NO:74;
(xxi) a heavy chain comprising the amino acid sequence of SEQ ID NO:92 and a light chain comprising the amino acid sequence SEQ ID NO:71;
(xxii) a heavy chain comprising the amino acid sequence of SEQ ID NO: 89 and a light chain comprising the amino acid sequence SEQ ID NO:71;
(xxiii) a heavy chain comprising the amino acid sequence of SEQ ID NO:90 and a light chain comprising the amino acid sequence SEQ ID NO:31;
(xxiv) a heavy chain comprising the amino acid sequence of SEQ ID NO:91 and a light chain comprising the amino acid sequence SEQ ID NO:71;
(xxv) a heavy chain comprising the amino acid sequence of SEQ ID NO: 85 and a light chain comprising the amino acid sequence SEQ ID NO:68;
(xxvi) a heavy chain comprising the amino acid sequence of SEQ ID NO: 86 and a light chain comprising the amino acid sequence SEQ ID NO:68;
(xxvii) a heavy chain comprising the amino acid sequence of SEQ ID NO:93 and a light chain comprising the amino acid sequence SEQ ID NO:100;
(xxviii)a heavy chain comprising the amino acid sequence of SEQ ID NO:94 and a light chain comprising the amino acid sequence SEQ ID NO:75;
(xxix) a heavy chain comprising the amino acid sequence of SEQ ID NO:95 and a light chain comprising the amino acid sequence SEQ ID NO:71;
(xxx) a heavy chain comprising the amino acid sequence of SEQ ID NO:96 and a light chain comprising the amino acid sequence SEQ ID NO:71;
(xxxi) a heavy chain comprising the amino acid sequence of SEQ ID NO:97 and a light chain comprising the amino acid sequence SEQ ID NO:71;
(xxxii) a heavy chain comprising the amino acid sequence of SEQ ID NO:98 and a light chain comprising the amino acid sequence SEQ ID NO:71;
(xxxiii) a heavy chain comprising the amino acid sequence of SEQ ID NO:83 and a light chain comprising the amino acid sequence SEQ ID NO:73;
(xxxiv) a heavy chain comprising the amino acid sequence of SEQ ID NO:87 and a light chain comprising the amino acid sequence SEQ ID NO:73;
(xxxv) a heavy chain comprising the amino acid sequence of SEQ ID NO:102 and a light chain comprising the amino acid sequence SEQ ID NO:101;
(xxxvi) a heavy chain comprising the amino acid sequence of SEQ ID NO:104 and a light chain comprising the amino acid sequence SEQ ID NO:103;
wherein the VH amino acid sequence is at least 90%, 95%, 97%, 98% or 99% identical thereto and the VL amino acid sequence is at least 90%, 95%, 97%, 98% or 99% identical thereto.
